(19) United States
(12) Patent Application Publication
(10) Pub. No.: US 2024/0158773 A1
(43) Pub. Date: May 16, 2024

(54) ENGINEERED BOTULINUM NEUROTOXIN A PROTEASE DOMAIN WITH IMPROVED EFFICACY

(71) Applicant: Children's Medical Center Cprporation, Boston, MA (US)

(72) Inventors: Min Dong, Weatogue, CT (US); Pyung-Gang Lee, Boston, MA (US)

(73) Assignee: Children's Medical Center Cprporation, Boston, MA (US)

(21) Appl. No.: 18/282,215

(22) PCT Filed: Mar. 14, 2022
(86) PCT No.: PCT/US2022/020217

§ 371 Date: Sep. 14, 2023

Related U.S. Application Data
(60) Provisional application No. 63/161,357, filed on Mar. 15, 2021.

(51) Int. Cl. C12N 9/52; A61K 8/66; A61K 38/48

(52) U.S. Cl. CPC C12N 9/52; A61K 8/66; A61K 38/4893; C12Y 304/24069

(57) ABSTRACT

Disclosed herein are modified Clostridial Botulinum neurotoxin (BoNT) polypeptides with a modified protease domains of Clostridial Botulinum serotype A1 or A2. Modifications include substitution amino acid mutations, isolated modified protease domains, chimeric molecules, pharmaceutical compositions, and methods of using the same are also disclosed.

### RELATED APPLICATIONS

This application claims the benefit under 35 U.S.C. § 119(e) of U.S. provisional application No. 63/161,357, titled “ENGINEERED BOTULINUM NEUROTOXIN A PROTEASE DOMAIN WITH IMPROVED EFFICACY,” filed Mar. 15, 2021, the entire contents of which are incorporated herein by reference.

### FEDERALLY SPONSORED RESEARCH

This invention was made with government support under R01NS080833 awarded by the National Institutes of Health. The government has certain rights in the invention.

### REFERENCE TO A SEQUENCE LISTING SUBMITTED AS A TEXT FILE

The instant application contains a Sequence Listing which has been submitted in ASCII format via EFS-Web and is hereby incorporated by reference in its entirety. Said ASCII copy, created on Mar. 14, 2022, is named C123370204WO00-SEQ-ARM and is 1,501,919 bytes in size.

### BACKGROUND

In recent years, Clostridial Botulinum neurotoxin (BoNT) have been widely used to treat a growing list of medical conditions: local injections of minute amount of toxins can attenuate neuronal activity in targeted regions, which can be beneficial in many medical conditions as well as for cosmetic purposes. To date, BoNT serotype A (BoNT/A) and BoNT serotype B (BoNT/B) are the only two BoNTs that are currently FDA-approved for use in humans. The major limitation is the generation of neutralizing antibodies in patients, which renders future treatment ineffective. Termination of BoNT usage often leaves patients with no other effective ways to treat/relieve their disorders. Adverse effects associated with BoNT use range from transient nonserious events such as ptosis and diplopia to life-threatening events even death.

### SUMMARY

Botulinum neurotoxins (BoNTs) are a family of bacterial toxins that target and enter nerve terminals and then act as a protease to cleave key proteins required for neurotransmitter release, thus inducing flaccid paralysis of muscles. Members of BoNTs, such as BoNT/A, have been utilized to treat a wide range of medical conditions as well as for cosmetic purposes of reducing wrinkles. A local injection of a tiny amount of BoNT/A can induce relaxation of muscles for 3-6 months in humans. However, an issue with clinical use of BoNT/A is that patients may generate neutralizing antibodies. Because the chance of generating neutralizing antibodies is directly related to injected doses, lowering the toxin therapeutic doses is highly desired, which means the efficacy of individual toxin molecule likely must be enhanced. Here, an approach was developed to enhance BoNT efficacy and therapeutic duration by “de-lysine” the lysine residues on the surface of the BoNT protease domain. Using this approach, protease domain mutants containing substitutions of surface lysine residues into arginine residues were systemically designed and tested in BoNT/A. A set of mutations were identified that significantly enhanced the efficacy than the wild-type protease domain. The enhanced efficacy of mutated BoNTs was validated in the context of full-length BoNT/A on both cultured neurons and in vivo in mouse models. These improved protease domains can be utilized to create a new generation of BoNT/A and other chimeric BoNT molecules for therapeutic uses, providing enhanced therapeutic efficacy, longer therapeutic duration, and lower chances of inducing neutralizing antibodies than currently available BoNTs.

In some aspects, a modified Clostridial Botulinum neurotoxin (BoNT) polypeptide comprises a modified protease domain of Clostridial Botulinum serotype A1 (BoNT/A1) comprising one or more arginine substitutions at positions corresponding to K11, K41, K212, K272, K289, K291, K299, K318, K335, K337, K340, K343, K356, K375, K381, Y387, M411 and K415 in SEQ ID NO: 1.

In some embodiments, the modified protease domain comprises one or more arginine substitutions at positions corresponding to K11, K335, K337, K343, K375, and K415 in SEQ ID NO: 1.

In some embodiments, the modified protease domain comprises one or more arginine substitutions at positions corresponding to K41, K318, and K340 in SEQ ID NO: 1.

In some embodiments, the modified protease domain comprises one or more arginine substitutions at positions corresponding to K289, K291, K299, and K381 in SEQ ID NO: 1.

In some embodiments, the modified protease domain comprises one or more arginine substitutions at positions corresponding to K212, K272, and K356 in SEQ ID NO: 1.

In some embodiments, the modified protease domain comprises one or more arginine substitutions at positions corresponding to K11, K41, K318, K335, K337, K340, K343, K375, and K415 in SEQ ID NO: 1.

In some embodiments, wherein the modified protease domain comprises one or more arginine substitutions at positions corresponding to K11, K289, K291, K299, K335, K337, K343, K375, K381, and K415 in SEQ ID NO: 1.

In some embodiments, wherein the modified protease domain comprises arginine substitutions at positions corresponding to K11, K289, K291, K299, K335, K337, K343, K375, K381, and K415 in SEQ ID NO: 1.

In some embodiments, the modified protease domain further comprises one or more arginine substitutions at positions corresponding to K41, K212, K272, K318, K340, and K356 in SEQ ID NO: 1.

In some embodiments, the modified protease domain comprises one or more arginine substitutions at positions corresponding to K11, K41, K289, K291, K299, K318, K335, K337, K340, K343, K375, K381, and K415 in SEQ ID NO: 1.

In some embodiments, wherein the modified protease domain comprises arginine substitutions at positions corresponding to K11, K41, K289, K291, K299, K318, K335, K337, K340, K343, K375, K381 and K415 in SEQ ID NO: 1.

In some embodiments, wherein the modified protease domain comprises one or more arginine substitutions at positions corresponding to K11, K41, K212, K289, K291, K299, K318, K335, K337, K340, K343, K356, K375, K381, and K415 in SEQ ID NO: 1.

In some embodiments, the modified protease domain comprises arginine substitutions at positions corresponding to K11, K41, K212, K289, K291, K299, K318, K335, K337, K340, K343, K356, K375, K381, and K415 in SEQ ID NO: 1.

In some embodiments, wherein the modified protease domain comprises one or more arginine substitutions at positions corresponding to K11, K41, K212, K272, K289, K291, K299, K318, K335, K337, K340, K343, K356, K375, K381, and K415 in SEQ ID NO: 1. In some embodiments, the modified protease domain comprises arginine substitutions at positions corresponding to K11, K41, K212, K272, K289, K291, K299, K318, K335, K337, K340, K343, K356, K375, K381, and K415 in SEQ ID NO: 1.

In some embodiments, wherein the modified protease domain comprises an arginine substitution of K11R in SEQ ID NO: 1.

In some embodiments, the modified protease domain comprises an arginine substitution of K41R in SEQ ID NO: 1.

In some embodiments, the modified protease domain comprises an arginine substitution of K212R in SEQ ID NO: 1.

In some embodiments, the modified protease domain comprises an arginine substitution of K272R in SEQ ID NO: 1.

In some embodiments, the modified protease domain comprises an arginine substitution of K289R in SEQ ID NO: 1.

In some embodiments, the modified protease domain comprises an arginine substitution of K291R in SEQ ID NO: 1.

In some embodiments, the modified protease domain comprises an arginine substitution of K299R in SEQ ID NO: 1.

In some embodiments, the modified protease domain comprises an arginine substitution of K318R in SEQ ID NO: 1.

In some embodiments, the modified protease domain comprises an arginine substitution of K335R in SEQ ID NO: 1.

In some embodiments, the modified protease domain comprises an arginine substitution of K337R in SEQ ID NO: 1.

In some embodiments, the modified protease domain comprises an arginine substitution of K340R in SEQ ID NO: 1.

In some embodiments, the modified protease domain comprises an arginine substitution of K343R in SEQ ID NO: 1.

In some embodiments, the modified protease domain comprises an arginine substitution of K356R in SEQ ID NO: 1.

In some embodiments, the modified protease domain comprises an arginine substitution of K375R in SEQ ID NO: 1.

In some embodiments, the modified protease domain comprises an arginine substitution of K381R in SEQ ID NO: 1.

In some embodiments, the modified protease domain comprises an arginine substitution of Y387R in SEQ ID NO: 1.

In some embodiments, the modified protease domain comprises an arginine substitution of M411R in SEQ ID NO: 1.

In some embodiments, the modified protease domain comprises an arginine substitution of K415R in SEQ ID NO: 1.

In some embodiments, the modified protease domain comprises an amino acid sequence that is at least 80% identical to the amino acid sequence of SEQ ID NO: 88-120 In some embodiments, the modified protease domain comprises the amino acid sequence of any one of SEQ ID NOs: 88-120.

In some embodiments, the modified BoNT polypeptide further comprises a translocation domain from BoNT/A1.

In some embodiments, the modified BoNT polypeptide comprises an amino acid sequence that is at least 80% identical to the amino acid sequence of SEQ ID NO: 45-77. In some embodiments, the modified BoNT polypeptide comprises the amino acid sequence of any one of SEQ ID NOs: 45-77.

In some embodiments, the modified BoNT polypeptide further comprises a receptor binding domain of BoNT/A1.

In some embodiments, the modified BoNT polypeptide comprises an amino acid sequence that is at least 80% identical to the amino acid sequence of any one of SEQ ID NOs: 2-34. In some embodiments, the modified BoNT polypeptide comprises the amino acid sequence of any one of SEQ ID NOs: 2-34.

In some embodiments, the receptor binding domain of BoNT/A1 comprises one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the one or more BoNT/A1 amino acid substitutions correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1.

In some embodiments, the receptor binding domain of BoNT/A1 comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises an amino acid sequence that is at least 80% identical to the amino acid sequence of any one of SEQ ID NOs: 131-165 fused to any one of SEQ ID NOs: 45-77.

In some embodiments, the modified BoNT polypeptide comprises an amino acid sequence that is at least 80% identical to the amino acid sequence of SEQ ID No: 177 or SEQ ID NO: 178. In some embodiments, the modified BoNT polypeptide comprises the amino acid sequence of SEQ ID NO: 177 or SEQ ID NO: 178.

In some embodiments, the modified BoNT polypeptide further comprises a receptor binding domain from BoNT/A2.

In some embodiments, the receptor binding domain comprises one or more amino acid substitutions at positions corresponding to 915, 923, 1090, 1103, 1117, 1156, 1170, 1227, 1254, 1255, or 1256 in SEQ ID NO: 35. In some embodiments, the receptor binding domain comprises one or more amino acid substitutions correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide comprises an amino acid sequence that is at least 80% identical to the amino acid sequence of SEQ ID NO: 166-176 or 204 fused to any one of SEQ ID NO: 45-77.

In some embodiments, the modified BoNT polypeptide comprises the amino acid sequence of SEQ ID NO: 166-176 or 204 fused to any one of SEQ ID NO: 45-77.

In some embodiments, the modified BoNT polypeptide further comprising a receptor binding domain from a second BoNT. In some embodiments, the modified BoNT polypeptide further comprising a receptor binding domain from a second BoNT is of serotype B, C, D, E, F, G, H, or En.

In some embodiments, the modified BoNT polypeptide comprises an amino acid sequence that is at least 80% identical to the amino acid sequence of any one of SEQ ID NO: 179-186 fused to any one of SEQ ID NO: 45-77.

In some embodiments, the modified BoNT polypeptide comprises the amino acid sequence of SEQ ID NO: 179-186 fused to any one of SEQ ID NO: 45-77.

In some embodiments, the modified BoNT polypeptide comprises the amino acid sequence of SEQ ID NO: 203.

In some embodiments, the modified BoNT polypeptide comprising a modified protease domain of BoNT/A1 and translocation domain of BoNT/A1 further comprising a modified linker region. In some embodiments, the modified linker region comprises a protease cleavage site. In some embodiments, the protease cleavage site comprises the amino acid sequence of any one of SEQ ID NO: 206-212.

In some aspects, a modified Clostridial Botulinum neurotoxin (BoNT) polypeptide comprises a modified protease domain of Clostridial Botulinum serotype A2 (BoNT/A2) comprising one or more arginine substitutions at positions corresponding to K11, K289, K291, K299, K335, K337, and K343 in SEQ ID NO: 2.

In some embodiments, the modified protease domain of Clostridial Botulinum serotype A2 (BoNT/A2) comprises arginine substitutions at positions corresponding to K11, K289, K291, K299, K335, K337, and K343 in SEQ ID NO: 2.

In some embodiments, the modified protease domain of BoNT/A2 comprises an arginine substitution of K11R in SEQ ID NO: 2.

In some embodiments, the modified protease domain of BoNT/A2 comprises an arginine substitution of K289R in SEQ ID NO: 2.

In some embodiments, the modified protease domain of BoNT/A2 comprises an arginine substitution of K291R in SEQ ID NO: 2.

In some embodiments, wherein the modified protease domain of BoNT/A2 comprises an arginine substitution of K299R in SEQ ID NO: 2.

In some embodiments, wherein the modified protease domain of BoNT/A2 comprises an arginine substitution of K335R in SEQ ID NO: 2.

In some embodiments, wherein the modified protease domain of BoNT/A2 comprises an arginine substitution of K337R in SEQ ID NO: 2.

In some embodiments, wherein the modified protease domain of BoNT/A2 comprises an arginine substitution of K343R in SEQ ID NO: 2.

In some embodiments, the modified protease domain of BoNT/A2 comprises an amino acid sequence that is at least 80% identical to the amino acid sequence of SEQ ID NO: 122-129. In some embodiments, the modified protease binding domain comprises the amino acid sequence of any one of SEQ ID NOs: 122-129.

In some embodiments, the modified BoNT polypeptide further comprises a translocation domain from BoNT/A2.

In some embodiments, the modified BoNT polypeptide comprises an amino acid sequence that is at least 80% identical to the amino acid sequence of SEQ ID NO: 79-86. In some embodiments, the modified BoNT polypeptide comprises the amino acid sequence of any one of SEQ ID NOs: 79-86.

In some embodiments, the modified BoNT polypeptide further comprising a receptor binding domain of BoNT/A1.

In some embodiments, the modified BoNT polypeptide comprises an amino acid sequence that is at least 80% identical to the amino acid sequence of any one of SEQ ID NOs: 36-43. In some embodiments, the modified BoNT polypeptide comprises the amino acid sequence of any one of SEQ ID NOs: 36-43.

In some embodiments, the receptor binding domain of BoNT/A1 comprises one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the one or more amino acid substitutions in the receptor binding domain of BoNT/A1 correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1.

In some embodiments, the receptor binding domain of BoNT/A1 comprises an amino acid substitution at a position corresponding to 1156 or 1232 in SEQ ID NO: 1. In some embodiments, the amino acid substitution in the receptor binding domain of BoNT/A1 corresponds to R1156M or T1232R in SEQ ID NO: 1.

In some embodiments, the modified BoNT polypeptide comprises an amino acid sequence that is at least 80% identical to the amino acid sequence of any one of SEQ ID NOs: 131-165 or 205 fused to any one of SEQ ID NOs: 79-86. In some embodiments, the modified BoNT polypeptide comprises an amino acid sequence of any one of SEQ ID NOs: 131-165 or 205 fused to any one of SEQ ID NOs: 79-86.

In some embodiments, the modified BoNT polypeptide further comprises a receptor binding domain from BoNT/A2.

In some embodiments, the modified receptor binding domain comprises one or more amino acid substitutions at positions corresponding to 915, 923, 1090, 1103, 1117, 1156, 1170, 1227, 1254, 1255, or 1256 in SEQ ID NO: 35. In some embodiments, the one or more the amino acid substitutions in the modified receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises an amino acid sequence that is at least 80% identical to the amino acid sequence of SEQ ID NO: 166-176 fused to any one of SEQ ID NO: 79-86. In some embodiments, the modified BoNT polypeptide comprises the amino acid sequence of any one of SEQ ID NO: 166-176 fused to any one of SEQ ID NO: 79-86.

In some embodiments, the modified BoNT polypeptide further comprises a receptor binding domain from a second BoNT. In some embodiments, the receptor binding domain from a second BoNT is of serotype B, C, D, E, F, G, H, or En.

In some embodiments, the modified BoNT polypeptide comprises an amino acid sequence that is at least 80% identical to the amino acid sequence of any one of SEQ ID NO: 179-186 fused to any one of SEQ ID NO: 79-86.

In some embodiments, the modified BoNT polypeptide comprises the amino acid sequence of any one of SEQ ID NO: 179-186 fused to any one of SEQ ID NO: 79-86.

In some embodiments, the modified BoNT polypeptide comprising a modified protease domain of BoNT/A2 and a translocation domain of BoNT/A2 further comprises a modified linker region. In some embodiments, the modified linker region comprises a protease cleavage site. In some embodiments, the protease cleavage site comprises the amino acid sequence of any one of SEQ ID NO: 206-212.

In some aspects, a nucleic acid molecule comprises a polynucleotide encoding any one of the modified BoNTs described herein. In some embodiments, a nucleic acid vector comprises the nucleic acid molecules encoding for any one of the BoNTs described herein. In some embodiments, a cell comprises the nucleic acid molecule or nucleic acid vector encoding for any one of the BoNTs described herein. In some embodiments, a cell expresses any one of the modified BoNT polypeptides described herein.

In some embodiments, a method of producing a modified BoNT polypeptide is performed, the method comprising the steps of culturing the cell comprising a nucleic acid molecule or nucleic acid vector encoding any one of the BoNT polypeptide described herein, wherein the cell expresses any on of the BoNT polypeptide, in conditions wherein the modified BoNT polypeptide is produced. In some embodiments, the method of producing a modified BoNT further comprises recovering the modified BoNT polypeptide from the culture.

In some aspects, the disclosure provides a pharmaceutical composition comprising any one of the modified BoNT polypeptides described herein. In some embodiments, the pharmaceutical composition comprising any one of the modified BoNT polypeptides described herein further comprises a pharmaceutically acceptable excipient.

In some embodiments, the disclosure provides a kit comprising a pharmaceutical composition described herein and directions for therapeutic administration of the pharmaceutical composition.

In some aspects, the disclosure provides methods of treating a condition, the method comprising administering a therapeutically effective amount of any one of the modified BoNTs described herein, or the pharmaceutical composition comprising the modified BoNTs described herein to a subject to treat the condition. In some embodiments, the condition is associated with overactive neurons or glands. In some embodiments, the condition is selected from the group consisting of: spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, dermatological or aesthetic/cosmetic conditions, obesity/reduced appetite, depression. In some embodiments, the condition is not associated with unwanted neuronal activity. In some embodiments, the condition is selected from the group consisting of: psoriasis, allergy, haemophagocytic lymphohistiocytosis, and alcoholic pancreatic disease. In some embodiments, the administering is via injection to where unwanted neuronal activity is present. In some embodiments, any one of the modified BoNT polypeptides described herein, or the any one of the pharmaceutical compositions described here are used in treating a condition associated with unwanted neuronal activity.

In some embodiments, any one of the modified BoNT polypeptides described herein, or the any one of the pharmaceutical compositions described here are used in medicine. In some embodiments, any one of the modified BoNT polypeptides described herein, or the any one of the pharmaceutical compositions described here are used cosmetics.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIGS. 1A-1B show the schematic view of BoNT action and its domains. FIG. 1A shows BoNTs are composed of a light chain and a heavy chain, connected via a disulfide bond. The heavy chain can be further divided into two domains: the translocation domain (H_{N}) and the receptor binding domain (H_{C}). FIG. 1B shows a schematic view of BoNT actions: BoNTs recognize neurons by binding to their specific receptors, enter neurons via receptor-mediated endocytosis, the light chains of BoNTs then translocate across endosomal membranes into the cytosol, where these light chains act as proteases to cleave target host proteins. Panel A is adapted from Arnon, S. et al, JAMA, 285:1059, 2001²⁵.

FIGS. 2A-2C shows the strategy for selecting amino acid substitutions. FIG. 1A shows crystal structure of LC/A1 complexed with SNAP25 fragment (PDB ID: 1XTG) denoted with all 35 lysines as mutation sites. FIG. 2B shows sequence alignment of the LCs of BoNT/A1-A8 with the selected lysine mutation sites outlined. FIG. 2C shows a list of all 35 lysines in A subtypes and the non-lysine residues in the corresponding sites. The corresponding residues with arginine (or non-K and non-R residues) in any A subtypes are marked with asterisks (or double asterisks).

FIGS. 3A-3D shows locations and groups lysines selected for arginine substitution, a scheme for BoNT production by Sortase ligation, proper activation of BoNTs, and measured protease activity of BoNTs on SNAP25. FIG. 3A shows crystal structure of BoNT/A1 labeled with 16 non-conserved lysines are shown, and the lysines were divided into three groups to generate five different KR mutants: KR6, KR9, KR10, KR13 and KR16. Group 1 residues are involved in SNAP25 binding, while Group 2 residues are distantly located from SNAP25 binding site. Three Group 3 residues are located between LC and HC. FIG. 3B shows a schematic drawing of sortase ligation: A1-LC-H_{N} has a sortase tag (LPETG) on its C-terminus which can be ligated with N-terminal free glycine of A1-H_{C} by sortase. After ligation, the full-length protein should be activated by thrombin to cleave a linker between LC and H_{N} to obtain its functional activity. FIG. 3C shows SDS-PAGE gel image showing the ligation and activation. The full-length ligated toxins (fl-BoNT/A) can be shown at ˜150 kDa without treatment of 2-mercaptoethanol (2-ME), but separated into LC and H_{C} with 2-ME. All the mutant toxins tested in this study were ligated and analyzed using this method. FIG. 3D shows cultured rat cortical neurons were incubated with ligated full-length toxins for 12 hours. Cleaved and un-cleaved SNAP25 of neuron lysates were detected in an immunoblotting assay. SNAP25 cleavage by full-length BoNT/A1-KR6, KR9 and KR10 was slightly more efficient than WT. KR16 displayed notably enhanced SNAP25 cleavage compared to than WT.

FIGS. 4A-4E show evaluation of mutant BoNT toxins in vivo in mice using Digit Abduction Score (DAS) assays. The data shows that mutant BoNTs produce higher DAS scores than the wildtype BoNT at the same dosage. FIG. 4A shows a standard scoring for DAS assay. Score 0 means normal, while score 4 represents most severe paralysis. FIG. 4B shows DAS results of five KR mutants: KR6, KR9, KR10, KR13 and KR16 along with WT (control). All of these mutants showed enhanced activity compared to WT. The most enhanced mutant was KR10. KR10 paralysis lasted about twice as long as wildtype WT in the same dose and had a higher overall effect on paralysis than wildtype as measured by DAS. FIG. 4C shows DAS results of six KR11 mutants: KR11-41 (KR10+K41R), KR11-212, KR11-272, KR11-318, KR11-340 and KR11-356 along with KR10 and KR16 (controls). These mutants were evaluated to explore the optimal KR mutant between KR10 and KR16. All the KR11s mutants showed similar activity compared to KR10 and KR16. FIG. 4D shows DAS results of BoNT/A1 WT with three different injection doses: 15, 30 and 75 pg. WT BoNT/A1 injection elicited a dose-dependent response and lasted about a month. FIG. 4E shows DAS results of BoNT/A1 KR10 mutant with three different injection doses: 5, 10 and 25 pg. Based on comparison of FIGS. 4D and 4E, KR10 showed at least three times higher potency and longer duration in a similar dose (30 pg of WT vs. 25 pg of KR10) than WT.

FIGS. 5A-5B show evaluation of modified BoNTs comprising a KR10 modified protease domain and a modified or chimeric receptor binding domain using in vivo using DAS assays. FIG. 5A shows DAS results of KR10 mutants comprising a H_{C}/A1 WT (control) or a H_{C}/A1 mutant comprising R1156M or T1232R. FIG. 5B shows DAS results of a WT or KR10 LC-Hn boNT/A1 fused to the H_{C} of BoNT/A (30 pg). The KR10 mutant showed higher in vivo potency when it was ligated with another serotype H_{C}, H_{C}/B here.

### DETAILED DESCRIPTION OF CERTAIN EMBODIMENTS

Botulinum neurotoxins (BoNTs) are a family of bacterial toxins produced by clostridium bacteria, with seven well-established serotypes (BoNT/A-G)^{1,32-33} and two recently discovered BoNT-like toxins types X and EN described in US Patent Application Publications US20190136216 and US20200255481 (both incorporated herein by reference). They are one of the most dangerous potential bio-terrorism agents, classified as a “Category A” select agent by Center for Disease Control (CDC) of United States 31. These toxins are produced as a single polypeptide and can be separated by bacterial or host proteases into a light chain (LC, ˜50 kDa) and a heavy chain (HC, ˜100 kDa). The two chains remain connected via an inter-chain disulfide bond. The HC contains two sub-domains: the N-terminal H_{N} domain that mediates translocation of the LC across endosomal membranes, and the C-terminal H_{C} domain that mediates binding to receptors on neurons. The inter-chain disulfide bond is reduced once the LC translocates into the cytosol³⁴⁻³⁵. Released LC acts as a protease to specifically cleave a set of neuronal proteins: BoNT/A, C, and E cleave at distinct sites on a protein known as SNAP-25; BoNT/B, D, F, and G cleave at different sites on a vesicle protein VAMP; and BoNT/C also cleaves a transmembrane protein syntaxin 1^{1,32-33}. These three proteins form a complex, known as SNARE complex, which is essential for release of neurotransmitters³⁶⁻³⁷. Cleavage of any one of these three SNARE proteins blocks neurotransmitters release from neurons, thus paralyzing muscles. Recently discovered BoNT/X cleaves SNARE proteins like other BoNTs, but also cleaves non-canonical substrates VAMP4, VAMP5 and Ykt6. Recently discovered BoNT/EN cleaves VAMP1/2/3 and several other SNARE proteins including SNAP-25, SNAP-23, syntaxin 1B and syntaxin 4.

BoNTs are the most potent toxins known and cause the human and animal disease known as botulism³³. The major form of botulism is caused by ingesting food contaminated with BoNTs (food botulism). Other forms also exist such as infant botulism, which is due to colonization of the intestine by toxin-producing bacteria in infants. BoNTs are always produced together with another 150 kDa protein known as NTNHA (non-toxic non-hemagglutinin protein), which forms a pH-dependent complex with BoNTs and protects BoNTs from proteases in the gastrointestinal tract³⁸.

Because local injections of minute amounts of toxins can attenuate neuronal activity in targeted regions, BoNTs have been used to treat a growing list of medical conditions³⁻⁵, including muscle spasms, chronic pain, overactive bladder problems, as well as for cosmetic applications. The market for BoNTs has already surpassed $3 billion in 2018. Among the seven types of BoNT toxins, BoNT/A and BoNT/B are the two toxins that are currently FDA-approved for use in humans³⁻⁵. BoNT/A is the dominant type used for both medical and cosmetic applications, marketed as Botox from Allergan Inc., Dysport from IPSEN Inc., and Xeomin from Merz Inc. BoNT/B is marketed as Myobloc by USWorld Med.

As the application of BoNTs grows, limitations and adverse effects have been reported. The major limitation is the generation of neutralizing antibodies in patients, which renders future treatment ineffective⁶. Termination of BoNT usage often leaves patients with no other effective ways to treat or relieve their disorders. The probability of antibody responses is directly related to both toxin doses and the frequency of injection⁶. Therefore, this limitation mainly occurs in treating muscle spasms, which involves high dose of toxins. Consistently, antibody responses have not been observed in cosmetic applications, which use extremely low toxin doses⁶.

The major adverse effects are also often associated with treating muscle spasms, but not cosmetic applications. This is because the adverse effects are largely due to diffusion of toxins to other regions of the body and the possibility of toxin diffusion is directly related to injected doses. The adverse effects range from transient non-serious events such as ptosis and diplopia to life-threatening events even death^{7,8}. In a petition letter filed in 2008 by Dr. Sidney Wolfe to FDA, a total of 180 serious adverse events, including 16 deaths have been documented. As a result, FDA now requires the “Black box warning” on all BoNT products, highlighting the risk of the spread of toxins, following similar warnings issued by the European Union.

Because both the generation of neutralizing antibodies and toxin diffusion are directly related to injected doses, lowering toxin doses (while maintaining the same levels of toxin activity) is highly desired, which means the efficacy of individual toxin molecules to induce local muscle paralysis has to be enhanced. Such modified BoNTs with improved local efficacy would also reduce any potential off-target effects due to toxin diffusion to other regions.

The action of BoNTs has three major steps: (1) receptor binding: these toxins target motor nerve terminals by first binding specifically to their receptors expressed in neurons; (2) translocation: after binding to receptors, BoNTs enter cells via receptor-mediated endocytosis into endosomes, and the low pH conditions within endosomes then induce conformational changes of toxin, resulting in its penetration of endosomal membrane and release of its protease domain into the cytosol of neurons; (3) substrate cleavage: within the cytosol of neurons, the released protease domain of BoNTs then cleave proteins required for synaptic transmission, therefore blocking neurotransmission². Corresponding to these three steps of action, BoNTs are composed of three functional domains²: (1) the C-terminal receptor-binding domain (H_{C}, ˜50 kDa); (2) the membrane translocation domain in the middle (H_{N}, ˜50 kDa); (3) the N-terminal protease domain (also known as light chain, LC, ˜50 kDa). The H_{C} and H_{N} together form the heavy chain (HC, ˜100 kDa).

Herein an approach was developed to enhance BoNT efficacy and therapeutic duration by “de-lysine” the lysine residues on the surface of the BoNT protease domain. Using this approach, BoNT/A protease domain mutants containing substitutions of primarily surface lysine residues into arginines were systemically designed and tested. Multiple sets of mutations were identified that significantly enhanced BoNT/A efficacy compared to BoNTs with a wild-type protease domain. Mutant BoNT enhanced efficacy was validated in the context of full-length BoNT/A on both cultured neurons and in vivo in mouse models. These improved protease domains can be utilized to create a new generation of BoNT/A and other chimeric BoNT molecules for therapeutic uses, providing enhanced therapeutic efficacy, longer therapeutic duration, and lower chances of inducing neutralizing antibodies than currently available BoNTs.

Accordingly, some aspects of the present disclosure provide modified Clostridial Botulinum neurotoxins (BoNT) comprising a modified protease domain of Clostridial Botulinum serotype A (BoNT/A). In some embodiments, a BoNT comprising the modified protease domain of a BoNT/A increases catalytic activity of the modified BoNT compared to a BoNT comprising a wildtype BoNT/A protease domain. In some embodiments, a BoNT comprising the modified protease domain of BoNT/A reduces systemic toxicity at a dosage that induces the same degree of local paralysis as a BoNT comprising a wildtype BoNT/A protease domain. In some embodiments, a BoNT comprising the modified protease domain of a BoNT/A increases local paralysis compared to a BoNT comprising a wildtype BoNT/A protease domain.

As used herein, the term “Clostridial Botulinum neurotoxin (BoNT)” encompasses any polypeptide or fragment from a Botulinum neurotoxin. In some embodiments, the term BoNT refers to a full-length BoNT. In some embodiments, the term BoNT refers to a fragment of the BoNT that can execute the overall cellular mechanism whereby a BoNT enters a neuron and inhibits neurotransmitter release. In some embodiments, the term BoNT simply refers to a fragment of the BoNT, without requiring the fragment to have any specific function or activity. Other terms that may be used throughout the present disclosure for “Clostridial Botulinum neurotoxins” may be BoNTs, Botulinum toxins, or C. Botulium toxins. It is to be understood that these terms are used interchangeably.

A “modified Clostridial Botulinum neurotoxin (BoNT)” encompasses a BoNT comprising any modifications in the amino acid sequence, e.g., truncation, addition, amino acid substitution, and any combination thereof. For example, a BoNT/A1 comprising amino acid substitution mutations at positions K11 or K41 corresponding to SEQ ID NO: 1 is a modified BoNT. In another example, a fragment or a domain of the full-length BoNT (e.g., the protease domain) is considered a modified BoNT. In some embodiments, a domain of the BoNT may also comprise amino acid substitution mutation(s), (e.g., a protease domain) comprising substitution mutation at positions corresponding to K11R or K41R of the full-length BoNT/A1 (SEQ ID NO: 1).

As used herein, the term “Clostridial Botulinum neurotoxin (BoNT) protease domain” or “LC” means a BoNT domain that can execute the enzymatic target modification step of the intoxication process. Thus, a BoNT protease domain specifically targets a C. botulinum toxin substrate and encompasses the proteolytic cleavage of a C. botulinum toxin substrate, such as, e.g., SNARE proteins like a SNAP-25 substrate, a VAMP substrate and a Syntaxin substrate. Some aspects of present disclosure relate to modified BoNT protease domains from serotype A (BoNT/A), that enhances the protease activity of the BoNT/A. BoNT/A has eight subtypes, BoNT/A1, BoNT/A2, BoNT/A3, BoNT/A4, BoNT/A5, BoNT/A6, BoNT/A7, and BoNT/A8. Thus, the present disclosure encompasses modified BoNT/A protease domain from all and any of the eight subtypes. It is appreciated that when “BoNT/A” is referred to, it encompasses all the subtypes of BoNT/A. In some embodiments, a “modified BoNT/A protease domain” comprises one or more novel amino acid substitution mutations described in the present disclosure.

Some aspects of present disclosure relate to modified BoNT protease domains from serotype A1 (BoNT/A1), that enhance the protease activity of BoNT/A1. In some embodiments, a “modified BoNT/A1 protease domain” comprises one or more novel amino acid substitution mutations described in the present disclosure. In some embodiments, the modified protease domain of BoNT/A1 comprises about amino acids 1-441 of SEQ ID NO: 1. It is to be understood that the border of the BoNT/A1 protease binding domain fragment may vary by 1-10 amino acids. For example, the modified BoNT/A1 protease domain that may be used for the chimeric toxin may comprise amino acids 1-431, 1-432, 1-433, 1-434, 1-435, 1-436, 1-437, 1-438, 1-439, 1-440, 1-441, 1-442, 1-443, 1-444, 1-445, 1-446, 1-447, 1-448, 1-449 or 1-450 of SEQ ID NO: 1.

Some aspects of present disclosure relate to modified BoNT protease domains from serotype A2 (BoNT/A2), that enhances the protease activity of the BoNT/A2. In some embodiments, a “modified BoNT/A2 protease domain” comprises one or more novel amino acid substitution mutations described in the present disclosure. In some embodiments, the modified protease domain of BoNT/A2 comprises about amino acids 1-441 of SEQ ID NO: 2. It is to be understood that the border of the BoNT/A2 protease domain fragment may vary by 1-10 amino acids. For example, the modified BoNT/A2 protease domain that may be used for the chimeric toxin may comprise amino acids 1-431, 1-432, 1-433, 1-434, 1-435, 1-436, 1-437, 1-438, 1-439, 1-440, 1-441, 1-442, 1-443, 1-444, 1-445, 1-446, 1-447, 1-448, 1-449 or 1-450 of SEQ ID NO: 2.

As used herein, the term “Clostridial Botulinum neurotoxin (BoNT) translocation domain” or “Hn” means a BoNT domain that can execute the translocation step of the intoxication process that mediates BoNT light chain translocation. Thus, an Hn facilitates the movement of a BoNT light chain across a membrane into the cytoplasm of a cell. Non-limiting examples of a Hn include a BoNT/A Hn, a BoNT/B Hn, a BoNT/Cl Hn, a BoNT/D Hn, a BoNT/E Hn, a BoNT/F Hn, a BoNT/G Hn, a BoNT/H Hn, and a BoNT/En Hn.

As used herein, the term “Clostridial Botulinum neurotoxin (BoNT) receptor-binding domain” is synonymous with “H_{C} domain” and “H_{C}”, and means any naturally occurring BoNT receptor binding domain that can execute the cell binding step of the intoxication process, including, e.g., the binding of the BoNT to a BoNT-specific receptor system located on the plasma membrane surface of a target cell.

Some aspects of present disclosure relate to modified BoNT receptor binding domains from serotype A1 (BoNT/A1), that enhance the binding of the BoNT/A1 to a cell, e.g., neurons or a BoNT/A1 receptor. Thus, the present disclosure encompasses modified BoNT/A1 receptor binding domain from all and any of the eight subtypes. In some embodiments, a “modified BoNT/A1 receptor binding domain” comprises amino acid substitution mutations described in the present disclosure and in U.S. Application 63/128,758, which is incorporated by reference herein in its entirety. In some embodiments, the receptor binding domain of BoNT/A1 comprises about amino acids 873-1296 of SEQ ID NO: 1. It is to be understood that the border of the BoNT/A1 receptor binding domain fragment may vary by 1-10 amino acids. For example, the BoNT/A1 receptor binding domain that may be used for the chimeric toxin may comprise amino acids 863-1296, 864-1296, 865-1296, 866-1296, 867-1296, 868-1296, 869-1296, 870-1296, 871-1296, 872-1296, 873-1296, 874-1296, 875-1296, 876-1296, 877-1296, 878-1296, 879-1296, 880-1296, 881-1296, 882-1296, 883-1296 of SEQ ID NO: 1.

Some aspects of present disclosure relate to modified BoNT receptor binding domains from serotype A2 (BoNT/A2), that enhances the binding of the BoNT/A2 to a cell, e.g., neurons or a BoNT/A2 receptor. Thus, the present disclosure encompasses modified BoNT/A2 receptor binding domain from all and any of the eight subtypes. In some embodiments, a “modified BoNT/A2 receptor binding domain” comprises amino acid substitution mutations described in the present disclosure and in U.S. Application 63/128,758, which is incorporated by reference herein in its entirety. In some embodiments, the modified receptor binding domain of BoNT/A2 comprises about amino acids 873-1296 of SEQ ID NO: 2. It is to be understood that the border of the BoNT/A2 receptor binding domain fragment may vary by 1-10 amino acids. For example, the modified BoNT/A2 receptor binding domain that may be used for the chimeric toxin may comprise amino acids 863-1296, 864-1296, 865-1296, 866-1296, 867-1296, 868-1296, 869-1296, 870-1296, 871-1296, 872-1296, 873-1296, 874-1296, 875-1296, 876-1296, 877-1296, 878-1296, 879-1296, 880-1296, 881-1296, 882-1296, 883-1296 of SEQ ID NO: 2.

In some aspects, the modified BoNT polypeptide is a chimeric toxin, wherein receptor binding and transmembrane domains are from a serotype selected from the group consisting of B, C, D, E, F, G, H or En and combinations thereof and the modified protease domain comprises any one of the BoNT/A1 or BoNT/A2 modified protease domains described herein. In some embodiments the receptor binding and translocation domains may be fused with any one of the modified protease domains of BoNT/A1 or BoNT/A2.

In some aspects, the modified BoNT polypeptide is a chimeric toxin, wherein receptor binding domain is from a serotype selected from the group consisting of B, C, D, E, F, G, H or En, the transmembrane domain in from any one of the BoNT/A serotypes (e.g. BoNT/A1 or BoNT/A2) and the modified protease domain comprises any one of the BoNT/A1 or BoNT/A2 modified protease domains described herein. In some embodiments, any one of the receptor binding domains may be fused with any one of the modified protease and transmembrane domains of BoNT/A1 or BoNT/A2.

In some embodiments, the modified BoNT polypeptide is a chimeric toxin, wherein the receptor binding domain comprise the BoNT/B serotype. The receptor binding domain of BoNT/B comprises about amino acids 857-1291 of SEQ ID NO: 139. It is to be understood that the border of the BoNT/B receptor binding domain may vary by 1-10 amino acids. For example, the BoNT/B receptor binding domain that may be used for the chimeric toxin may comprise amino acids 847-1291, 848-1291, 849-1291, 850-1291, 851-1291, 852-1291, 853-1291, 854-1291, 855-1291, 856-1291, 857-1291, 858-1291, 859-1291, 860-1291, 861-1291, 862-1291, 863-1291, 864-1291, 865-1291, 866-1291, or 867-1291 of SEQ ID NO: 139.

In some embodiments, the modified BoNT polypeptide is a chimeric toxin, wherein receptor binding domain comprise the BoNT/C serotype. The receptor binding domain of BoNT/C comprises about amino acids 870-1291 of SEQ ID NO: 140. It is to be understood that the border of the BoNT/C receptor binding domain may vary by 1-10 amino acids. For example, the BoNT/C receptor binding domain that may be used for the chimeric toxin may comprise amino acids 860-1291, 861-1291, 862-1291, 863-1291, 864-1291, 865-1291, 866-1291, 867-1291, 868-1291, 869-1291, 870-1291, 871-1291, 872-1291, 873-1291, 874-1291, 875-1291, 876-1291, 877-1291, 878-1291, 879-1291, or 880-1291 of SEQ ID NO: 140.

In some embodiments, the modified BoNT polypeptide is a chimeric toxin, wherein receptor binding domain comprise the BoNT/D serotype. The receptor binding domain of BoNT/D comprises about amino acids 1-862 of SEQ ID NO: 141. It is to be understood that the border of the BoNT/D receptor binding domain may vary by 1-10 amino acids. For example, the BoNT/D receptor binding domain that may be used for the chimeric toxin may comprise amino acids 852-1276, 853-1276, 854-1276, 855-1276, 856-1276, 857-1276, 858-1276, 859-1276, 860-1276, 861-1276, 862-1276, 863-1276, 864-1276, 865-1276, 866-1276, 867-1276, 868-1276, 869-1276, 870-1276, 871-1276, 872-1276 of SEQ ID NO: 141.

In some embodiments, the modified BoNT polypeptide is a chimeric toxin, wherein receptor binding domain comprise the BoNT/E serotype. The receptor binding domain of BoNT/E comprises about amino acids 844-1252 of SEQ ID NO: 142. It is to be understood that the border of the BoNT/E receptor binding domain may vary by 1-10 amino acids. For example, the BoNT/E receptor binding domain that may be used for the chimeric toxin may comprise amino acids 834-1252, 835-1252, 836-1252, 837-1252, 838-1252, 839-1252, 840-1252, 841-1252, 842-1252, 843-1252, 844-1252, 845-1252, 846-1252, 847-1252, 848-1252, 849-1252, 850-1252, 851-1252, 852-1252, 853-1252, 854-1252 of SEQ ID NO: 142.

In some embodiments, the modified BoNT polypeptide is a chimeric toxin, wherein receptor binding domain comprise the BoNT/F serotype. The receptor binding domain of BoNT/F comprises about amino acids 863-1278 of SEQ ID NO: 143. It is to be understood that the border of the BoNT/F receptor binding domain may vary by 1-10 amino acids. For example, the BoNT/F receptor binding domain that may be used for the chimeric toxin may comprise amino acids 853-1278, 854-1278, 855-1278, 856-1278, 857-1278, 858-1278, 859-1278, 860-1278, 861-1278, 862-1278, 863-1278, 864-1278, 865-1278, 866-1278, 867-1278, 868-1278, 869-1278, 870-1278, 871-1278, 872-1278, or 873-1278 of SEQ ID NO: 143.

In some embodiments, the modified BoNT polypeptide is a chimeric toxin, wherein receptor binding domain comprise the BoNT/G serotype. The receptor binding domain of BoNT/G comprises about amino acids 862-1297 of SEQ ID NO: 144. It is to be understood that the border of the BoNT/G receptor binding domain may vary by 1-10 amino acids. For example, the BoNT/G receptor binding domain that may be used for the chimeric toxin may comprise amino acids 852-1297, 853-1297, 854-1297, 855-1297, 856-1297, 857-1297, 858-1297, 859-1297, 860-1297, 861-1297, 862-1297, 863-1297, 864-1297, 865-1297, 866-1297, 867-1297, 868-1297, 869-1297, 870-1297, 871-1297, or 872-1297 of SEQ ID NO: 144.

In some embodiments, the modified BoNT polypeptide is a chimeric toxin, wherein receptor binding domain comprise the BoNT/H serotype. The receptor binding domain of BoNT/H comprises about amino acids 858-1288 of SEQ ID NO: 145. It is to be understood that the border of the BoNT/H receptor binding domain may vary by 1-10 amino acids. For example, the BoNT/H receptor binding domain that may be used for the chimeric toxin may comprise amino acids 848-1288, 849-1288, 850-1288, 851-1288, 852-1288, 853-1288, 854-1288, 855-1288, 856-1288, 857-1288, 858-1288, 859-1288, 860-1288, 861-1288, 862-1288, 863-1288, 864-1288, 865-1288, 866-1288, 867-1288 or 868-1288 of SEQ ID NO: 145.

In some embodiments, the modified BoNT polypeptide is a chimeric toxin, wherein receptor binding domain comprise the BoNT/EN serotype as described in U.S. application Ser. No. 16/651,720 (incorporated herein by reference). The receptor binding domain of BoNT/EN comprises about amino acids 874-1279 of SEQ ID NO: 146. It is to be understood that the border of the BoNT/EN receptor binding domain may vary by 1-10 amino acids. For example, the BoNT/EN receptor binding domain that may be used for the chimeric toxin may comprise amino acids 864-1279, 865-1279, 866-1279, 867-1279, 868-1279, 869-1279, 870-1279, 871-1279, 872-1279, 873-1279, 874-1279, 875-1279, 876-1279, 877-1279, 878-1279, 879-1279, 880-1279, 881-1279, 882-1279, 883-1279 or 884-1279 of SEQ ID NO: 146.

Some aspects of the present disclosure provide modified Clostridial Botulinum neurotoxin (BoNT) polypeptides comprising a modified protease domain of Clostridial Botulinum serotype A1 (BoNT/A1). In some embodiments, the modified protease domain of BoNT/A1 comprises one or more arginine substitutions at positions corresponding to K11, K41, K212, K272, K289, K291, K299, K318, K335, K337, K340, K343, K356, K375, K381, Y387, M411 and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K11 in SEQ ID NO: 1. In some embodiments, the modified BoNT polypeptide comprises the amino acid sequence of SEQ ID NO: 88. In some embodiments, the modified BoNT polypeptide consists of the amino acid sequence of SEQ ID NO: 88.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to Kl 1 in SEQ ID NO: 1. In some embodiments, the modified BoNT polypeptide comprises the amino acid sequence of SEQ ID NO: 45. In some embodiments, the modified BoNT polypeptide consists of the amino acid sequence of SEQ ID NO: 45.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to Kl 1 in SEQ ID NO: 1. In some embodiments, the modified BoNT polypeptide comprises the amino acid sequence of SEQ ID NO: 2. In some embodiments, the modified BoNT polypeptide consists of the amino acid sequence of SEQ ID NO: 2.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K11 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K11 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 45 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K11 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K11 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K11 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 45. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 45. In some embodiments, the modified BoNT polypeptide comprises the amino acid sequence of any one of SEQ ID NOs: 187-194 having an arginine corresponding to K11 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K41 in SEQ ID NO: 1. In some embodiments, the modified BoNT polypeptide comprises the amino acid sequence of SEQ ID NO: 89. In some embodiments, the modified BoNT polypeptide consists of the amino acid sequence of SEQ ID NO: 89.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K41 in SEQ ID NO: 1. In some embodiments, the modified BoNT polypeptide comprises the amino acid sequence of SEQ ID NO: 46. In some embodiments, the modified BoNT polypeptide consists of the amino acid sequence of SEQ ID NO: 46.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K41 in SEQ ID NO: 1. In some embodiments, the modified BoNT polypeptide comprises the amino acid sequence of SEQ ID NO: 3. In some embodiments, the modified BoNT polypeptide consists of the amino acid sequence of SEQ ID NO: 3.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K41 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K41 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 46 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K41 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K41 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K41 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 46. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 46. In some embodiments, the modified BoNT polypeptide comprises the amino acid sequence of any one of SEQ ID NOs: 187-194 having an arginine corresponding to K41 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K212 in SEQ ID NO: 1. In some embodiments, the modified BoNT polypeptide comprises the amino acid sequence of SEQ ID NO: 90. In some embodiments, the modified BoNT polypeptide consists of the amino acid sequence of SEQ ID NO: 90.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K212 in SEQ ID NO: 1. In some embodiments, the modified BoNT polypeptide comprises the amino acid sequence of SEQ ID NO: 47. In some embodiments, the modified BoNT polypeptide consists of the amino acid sequence of SEQ ID NO: 47.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K212 in SEQ ID NO: 1. In some embodiments, the modified BoNT polypeptide comprises the amino acid sequence of SEQ ID NO: 4. In some embodiments, the modified BoNT polypeptide consists of the amino acid sequence of SEQ ID NO: 4.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K212 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K212 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 47 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K212 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K212 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K212 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 47. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 47. In some embodiments, the modified BoNT polypeptide comprises the amino acid sequence of any one of SEQ ID NOs: 187-194 having an arginine corresponding to K212 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K272 in SEQ ID NO: 1. In some embodiments, the modified BoNT polypeptide comprises the amino acid sequence of SEQ ID NO: 91. In some embodiments, the modified BoNT polypeptide consists of the amino acid sequence of SEQ ID NO: 91.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K272 in SEQ ID NO: 1. In some embodiments, the modified BoNT polypeptide comprises the amino acid sequence of SEQ ID NO: 48. In some embodiments, the modified BoNT polypeptide consists of the amino acid sequence of SEQ ID NO: 48.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K272 in SEQ ID NO: 1. In some embodiments, the modified BoNT polypeptide comprises the amino acid sequence of SEQ ID NO: 5. In some embodiments, the modified BoNT polypeptide consists of the amino acid sequence of SEQ ID NO: 5.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K272 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K272 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 48 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K272 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K272 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K272 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 48. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 48. In some embodiments, the modified BoNT polypeptide comprises the amino acid sequence of any one of SEQ ID NOs: 187-194 having an arginine corresponding to K272 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K289 in SEQ ID NO: 1. In some embodiments, the modified BoNT polypeptide comprises the amino acid sequence of SEQ ID NO: 92. In some embodiments, the modified BoNT polypeptide consists of the amino acid sequence of SEQ ID NO: 92.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K289 in SEQ ID NO: 1. In some embodiments, the modified BoNT polypeptide comprises the amino acid sequence of SEQ ID NO: 49. In some embodiments, the modified BoNT polypeptide consists of the amino acid sequence of SEQ ID NO: 49.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K289 in SEQ ID NO: 1. In some embodiments, the modified BoNT polypeptide comprises the amino acid sequence of SEQ ID NO: 6. In some embodiments, the modified BoNT polypeptide consists of the amino acid sequence of SEQ ID NO: 6.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K289 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K289 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 49 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K289 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K289 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K289 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 49. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 49. In some embodiments, the modified BoNT polypeptide comprises the amino acid sequence of any one of SEQ ID NOs: 187-194 having an arginine corresponding to K289 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K291 in SEQ ID NO: 1. In some embodiments, the modified BoNT polypeptide comprises the amino acid sequence of SEQ ID NO: 93. In some embodiments, the modified BoNT polypeptide consists of the amino acid sequence of SEQ ID NO: 93.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K291 in SEQ ID NO: 1. In some embodiments, the modified BoNT polypeptide comprises the amino acid sequence of SEQ ID NO: 50. In some embodiments, the modified BoNT polypeptide consists of the amino acid sequence of SEQ ID NO: 50.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K291 in SEQ ID NO: 1. In some embodiments, the modified BoNT polypeptide comprises the amino acid sequence of SEQ ID NO: 7. In some embodiments, the modified BoNT polypeptide consists of the amino acid sequence of SEQ ID NO: 7.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K291 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K291 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 50 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K291 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K291 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K291 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 50. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 50. In some embodiments, the modified BoNT polypeptide comprises the amino acid sequence of any one of SEQ ID NOs: 187-194 having an arginine corresponding to K291 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K299 in SEQ ID NO: 1. In some embodiments, the modified BoNT polypeptide comprises the amino acid sequence of SEQ ID NO: 94. In some embodiments, the modified BoNT polypeptide consists of the amino acid sequence of SEQ ID NO: 94.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K299 in SEQ ID NO: 1. In some embodiments, the modified BoNT polypeptide comprises the amino acid sequence of SEQ ID NO: 51. In some embodiments, the modified BoNT polypeptide consists of the amino acid sequence of SEQ ID NO: 51.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K299 in SEQ ID NO: 1. In some embodiments, the modified BoNT polypeptide comprises the amino acid sequence of SEQ ID NO: 8. In some embodiments, the modified BoNT polypeptide consists of the amino acid sequence of SEQ ID NO: 8.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K299 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K299 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 51 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K299 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K299 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K299 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 51. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 51. In some embodiments, the modified BoNT polypeptide comprises the amino acid sequence of any one of SEQ ID NOs: 187-194 having an arginine corresponding to K299 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K318 in SEQ ID NO: 1. In some embodiments, the modified BoNT polypeptide comprises the amino acid sequence of SEQ ID NO: 95. In some embodiments, the modified BoNT polypeptide consists of the amino acid sequence of SEQ ID NO: 95.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K318 in SEQ ID NO: 1. In some embodiments, the modified BoNT polypeptide comprises the amino acid sequence of SEQ ID NO: 52. In some embodiments, the modified BoNT polypeptide consists of the amino acid sequence of SEQ ID NO: 52.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K318 in SEQ ID NO: 1. In some embodiments, the modified BoNT polypeptide comprises the amino acid sequence of SEQ ID NO: 9. In some embodiments, the modified BoNT polypeptide consists of the amino acid sequence of SEQ ID NO: 9.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K318 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K318 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 52 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K318 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K318 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K318 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 52. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 52. In some embodiments, the modified BoNT polypeptide comprises the amino acid sequence of any one of SEQ ID NOs: 187-194 having an arginine corresponding to K318 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K335 in SEQ ID NO: 1. In some embodiments, the modified BoNT polypeptide comprises the amino acid sequence of SEQ ID NO: 96. In some embodiments, the modified BoNT polypeptide consists of the amino acid sequence of SEQ ID NO: 96.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K335 in SEQ ID NO: 1. In some embodiments, the modified BoNT polypeptide comprises the amino acid sequence of SEQ ID NO: 53. In some embodiments, the modified BoNT polypeptide consists of the amino acid sequence of SEQ ID NO: 53.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K335 in SEQ ID NO: 1. In some embodiments, the modified BoNT polypeptide comprises the amino acid sequence of SEQ ID NO: 10. In some embodiments, the modified BoNT polypeptide consists of the amino acid sequence of SEQ ID NO: 10.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K335 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at identical to SEQ ID NO: 78 fused to the amino acid sequence of any one of SEQ ID NO: 131-165 or 205, and comprises an arginine substitution corresponding to K335 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 53 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K335 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K335 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K335 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 53. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 53. In some embodiments, the modified BoNT polypeptide comprises the amino acid sequence of any one of SEQ ID NOs: 187-194 having an arginine corresponding to K335 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K337 in SEQ ID NO: 1. In some embodiments, the modified BoNT polypeptide comprises the amino acid sequence of SEQ ID NO: 97. In some embodiments, the modified BoNT polypeptide consists of the amino acid sequence of SEQ ID NO: 97.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K337 in SEQ ID NO: 1. In some embodiments, the modified BoNT polypeptide comprises the amino acid sequence of SEQ ID NO: 54. In some embodiments, the modified BoNT polypeptide consists of the amino acid sequence of SEQ ID NO: 54.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K337 in SEQ ID NO: 1. In some embodiments, the modified BoNT polypeptide comprises the amino acid sequence of SEQ ID NO: 11. In some embodiments, the modified BoNT polypeptide consists of the amino acid sequence of SEQ ID NO: 11.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K337 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K337 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 54 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K337 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K337 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K337 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 54. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 54. In some embodiments, the modified BoNT polypeptide comprises the amino acid sequence of any one of SEQ ID NOs: 187-194 having an arginine corresponding to K337 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K340 in SEQ ID NO: 1. In some embodiments, the modified BoNT polypeptide comprises the amino acid sequence of SEQ ID NO: 98. In some embodiments, the modified BoNT polypeptide consists of the amino acid sequence of SEQ ID NO: 98.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K340 in SEQ ID NO: 1. In some embodiments, the modified BoNT polypeptide comprises the amino acid sequence of SEQ ID NO: 55. In some embodiments, the modified BoNT polypeptide consists of the amino acid sequence of SEQ ID NO: 55.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K340 in SEQ ID NO: 1. In some embodiments, the modified BoNT polypeptide comprises the amino acid sequence of SEQ ID NO: 12. In some embodiments, the modified BoNT polypeptide consists of the amino acid sequence of SEQ ID NO: 12.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K340 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K340 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 55 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K340 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K340 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K340 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 55. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 55. In some embodiments, the modified BoNT polypeptide comprises the amino acid sequence of any one of SEQ ID NOs: 187-194 having an arginine corresponding to K340 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K343 in SEQ ID NO: 1. In some embodiments, the modified BoNT polypeptide comprises the amino acid sequence of SEQ ID NO: 99. In some embodiments, the modified BoNT polypeptide consists of the amino acid sequence of SEQ ID NO: 99.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K343 in SEQ ID NO: 1. In some embodiments, the modified BoNT polypeptide comprises the amino acid sequence of SEQ ID NO: 56. In some embodiments, the modified BoNT polypeptide consists of the amino acid sequence of SEQ ID NO: 56.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K343 in SEQ ID NO: 1. In some embodiments, the modified BoNT polypeptide comprises the amino acid sequence of SEQ ID NO: 13. In some embodiments, the modified BoNT polypeptide consists of the amino acid sequence of SEQ ID NO: 13.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K343 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K343 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 56 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K343 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K343 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K343 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 56. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 56. In some embodiments, the modified BoNT polypeptide comprises the amino acid sequence of any one of SEQ ID NOs: 187-194 having an arginine corresponding to K343 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K356 in SEQ ID NO: 1. In some embodiments, the modified BoNT polypeptide comprises the amino acid sequence of SEQ ID NO: 100. In some embodiments, the modified BoNT polypeptide consists of the amino acid sequence of SEQ ID NO: 100.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K356 in SEQ ID NO: 1. In some embodiments, the modified BoNT polypeptide comprises the amino acid sequence of SEQ ID NO: 57. In some embodiments, the modified BoNT polypeptide consists of the amino acid sequence of SEQ ID NO: 57.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K356 in SEQ ID NO: 1. In some embodiments, the modified BoNT polypeptide comprises the amino acid sequence of SEQ ID NO: 14. In some embodiments, the modified BoNT polypeptide consists of the amino acid sequence of SEQ ID NO: 14.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K356 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K356 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 57 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K356 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K356 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K356 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 57. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 57. In some embodiments, the modified BoNT polypeptide comprises the amino acid sequence of any one of SEQ ID NOs: 187-194 having an arginine corresponding to K356 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K375 in SEQ ID NO: 1. In some embodiments, the modified BoNT polypeptide comprises the amino acid sequence of SEQ ID NO: 101. In some embodiments, the modified BoNT polypeptide consists of the amino acid sequence of SEQ ID NO: 101.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K375 in SEQ ID NO: 1. In some embodiments, the modified BoNT polypeptide comprises the amino acid sequence of SEQ ID NO: 58. In some embodiments, the modified BoNT polypeptide consists of the amino acid sequence of SEQ ID NO: 58.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K375 in SEQ ID NO: 1. In some embodiments, the modified BoNT polypeptide comprises the amino acid sequence of SEQ ID NO: 15. In some embodiments, the modified BoNT polypeptide consists of the amino acid sequence of SEQ ID NO: 15.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K375 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K375 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 58 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K375 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K375 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K375 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 58. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 58. In some embodiments, the modified BoNT polypeptide comprises the amino acid sequence of any one of SEQ ID NOs: 187-194 having an arginine corresponding to K375 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K381 in SEQ ID NO: 1. In some embodiments, the modified BoNT polypeptide comprises the amino acid sequence of SEQ ID NO: 102. In some embodiments, the modified BoNT polypeptide consists of the amino acid sequence of SEQ ID NO: 102.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K381 in SEQ ID NO: 1. In some embodiments, the modified BoNT polypeptide comprises the amino acid sequence of SEQ ID NO: 59. In some embodiments, the modified BoNT polypeptide consists of the amino acid sequence of SEQ ID NO: 59.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K381 in SEQ ID NO: 1. In some embodiments, the modified BoNT polypeptide comprises the amino acid sequence of SEQ ID NO: 16. In some embodiments, the modified BoNT polypeptide consists of the amino acid sequence of SEQ ID NO: 16.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K381 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K381 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 59 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K381 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K381 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K381 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 59. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 59. In some embodiments, the modified BoNT polypeptide comprises the amino acid sequence of any one of SEQ ID NOs: 187-194 having an arginine corresponding to K381 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to Y387 in SEQ ID NO: 1. In some embodiments, the modified BoNT polypeptide comprises the amino acid sequence of SEQ ID NO: 103. In some embodiments, the modified BoNT polypeptide consists of the amino acid sequence of SEQ ID NO: 103.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to Y387 in SEQ ID NO: 1. In some embodiments, the modified BoNT polypeptide comprises the amino acid sequence of SEQ ID NO: 60. In some embodiments, the modified BoNT polypeptide consists of the amino acid sequence of SEQ ID NO: 60.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to Y387 in SEQ ID NO: 1. In some embodiments, the modified BoNT polypeptide comprises the amino acid sequence of SEQ ID NO: 17. In some embodiments, the modified BoNT polypeptide consists of the amino acid sequence of SEQ ID NO: 17.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to Y387 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to Y387 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 60 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to Y387 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to Y387 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to Y387 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 60. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 60. In some embodiments, the modified BoNT polypeptide comprises the amino acid sequence of any one of SEQ ID NOs: 187-194 having an arginine corresponding to Y387 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to M411 in SEQ ID NO: 1. In some embodiments, the modified BoNT polypeptide comprises the amino acid sequence of SEQ ID NO: 104. In some embodiments, the modified BoNT polypeptide consists of the amino acid sequence of SEQ ID NO: 104.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to M411 in SEQ ID NO: 1. In some embodiments, the modified BoNT polypeptide comprises the amino acid sequence of SEQ ID NO: 61. In some embodiments, the modified BoNT polypeptide consists of the amino acid sequence of SEQ ID NO: 61.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to M411 in SEQ ID NO: 1. In some embodiments, the modified BoNT polypeptide comprises the amino acid sequence of SEQ ID NO: 18. In some embodiments, the modified BoNT polypeptide consists of the amino acid sequence of SEQ ID NO: 18.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to M411 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to M411 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 61 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to M411 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to M411 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to M411 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 61. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 61. In some embodiments, the modified BoNT polypeptide comprises the amino acid sequence of any one of SEQ ID NOs: 187-194 having an arginine corresponding to M411 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K415 in SEQ ID NO: 1. In some embodiments, the modified BoNT polypeptide comprises the amino acid sequence of SEQ ID NO: 105. In some embodiments, the modified BoNT polypeptide consists of the amino acid sequence of SEQ ID NO: 105.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises an arginine substitution corresponding to K415 in SEQ ID NO: 1. In some embodiments, the modified BoNT polypeptide comprises the amino acid sequence of SEQ ID NO: 62. In some embodiments, the modified BoNT polypeptide consists of the amino acid sequence of SEQ ID NO: 62.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K415 in SEQ ID NO: 1. In some embodiments, the modified BoNT polypeptide comprises the amino acid sequence of SEQ ID NO: 19. In some embodiments, the modified BoNT polypeptide consists of the amino acid sequence of SEQ ID NO: 19.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises an arginine substitution corresponding to K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises an arginine substitution corresponding to K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that comprises SEQ ID NO: 62 fused to the amino acid sequence of any one of SEQ ID NO: 131-165 or 205, and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising an arginine substitution corresponding to K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising an arginine substitution corresponding to K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprises an arginine substitution corresponding to K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 62. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 62. In some embodiments, the modified BoNT polypeptide comprises the amino acid sequence of any one of SEQ ID NOs: 187-194 having an arginine corresponding to K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K335, K337, K343, K375, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K335, K337, K343, K375, and K415 in SEQ ID NO: 1. In some embodiments, the group of arginine substitutions at positions corresponding to K11, K335, K337, K343, K375, and K415 in SEQ ID NO: 1 is referred to as Group 0. In some embodiments, the modified BoNT polypeptide comprises the amino acid sequence of SEQ ID NO: 106. In some embodiments, the modified BoNT polypeptide consists of the amino acid sequence of SEQ ID NO: 106.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K335, K337, K343, K375, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K335, K337, K343, K375, and K415 in SEQ ID NO: 1. In some embodiments, the modified BoNT polypeptide comprises the amino acid sequence of SEQ ID NO: 63. In some embodiments, the modified BoNT polypeptide consists of the amino acid sequence of SEQ ID NO: 63.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K335, K337, K343, K375, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K335, K337, K343, K375, and K415 in SEQ ID NO: 1. In some embodiments, the modified BoNT polypeptide comprises the amino acid sequence of SEQ ID NO: 20. In some embodiments, the modified BoNT polypeptide consists of the amino acid sequence of SEQ ID NO: 20.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K335, K337, K343, K375, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K335, K337, K343, K375, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K335, K337, K343, K375, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 63 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K335, K337, K343, K375, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K335, K337, K343, K375, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K335, K337, K343, K375, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K335, K337, K343, K375, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 63. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 63. In some embodiments, the modified BoNT polypeptide comprises the amino acid sequence of any one of SEQ ID NOs: 187-194 having arginine substitutions at positions corresponding to one or more of K11, K335, K337, K343, K375, and K415 in SEQ ID NO: 1. In some embodiments, the modified BoNT polypeptide comprises the amino acid sequence of any one of SEQ ID NOs: 187-194 having arginine substitutions at positions corresponding to K11, K335, K337, K343, K375, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K41, K318, and K340 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K41, K318, and K340 in SEQ ID NO: 1. In some embodiments, the group of arginine substitutions at positions corresponding to K41, K318, and K340 in SEQ ID NO: 1 is referred to as Group 1. In some embodiments, the modified BoNT polypeptide comprises the amino acid sequence of SEQ ID NO: 107. In some embodiments, the modified BoNT polypeptide consists of the amino acid sequence of SEQ ID NO: 107.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K41, K318, and K340 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K41, K318, and K340 in SEQ ID NO: 1. In some embodiments, the modified BoNT polypeptide comprises the amino acid sequence of SEQ ID NO: 64. In some embodiments, the modified BoNT polypeptide consists of the amino acid sequence of SEQ ID NO: 64.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K41, K318, and K340 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K41, K318, and K340 in SEQ ID NO: 1. In some embodiments, the modified BoNT polypeptide comprises the amino acid sequence of SEQ ID NO: 21. In some embodiments, the modified BoNT polypeptide consists of the amino acid sequence of SEQ ID NO: 21.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K41, K318, and K340 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K41, K318, and K340 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K41, K318, and K340 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 64 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K41, K318, and K340 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K41, K318, and K340 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K41, K318, and K340 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K41, K318, and K340 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 64. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 64. In some embodiments, the modified BoNT polypeptide comprises the amino acid sequence of any one of SEQ ID NOs: 187-194 having arginine substitutions at positions corresponding to one or more of K41, K318, and K340 in SEQ ID NO: 1. In some embodiments, the modified BoNT polypeptide comprises the amino acid sequence of any one of SEQ ID NOs: 187-194 having arginine substitutions at positions corresponding to K41, K318, and K340 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K289, K291, K299, and K381 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K289, K291, K299, and K381 in SEQ ID NO: 1. In some embodiments, the group of arginine substitutions at positions corresponding to K289, K291, K299, and K381 in SEQ ID NO: 1 is referred to as Group 2. In some embodiments, the modified BoNT polypeptide comprises the amino acid sequence of SEQ ID NO: 108. In some embodiments, the modified BoNT polypeptide consists of the amino acid sequence of SEQ ID NO: 108.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K289, K291, K299, and K381 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K289, K291, K299, and K381 in SEQ ID NO: 1. In some embodiments, the modified BoNT polypeptide comprises the amino acid sequence of SEQ ID NO: 65. In some embodiments, the modified BoNT polypeptide consists of the amino acid sequence of SEQ ID NO: 65.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K289, K291, K299, and K381 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K289, K291, K299, and K381 in SEQ ID NO: 1. In some embodiments, the modified BoNT polypeptide comprises the amino acid sequence of SEQ ID NO: 22. In some embodiments, the modified BoNT polypeptide consists of the amino acid sequence of SEQ ID NO: 22.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K289, K291, K299, and K381 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K289, K291, K299, and K381 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K289, K291, K299, and K381 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 65 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K289, K291, K299, and K381 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K289, K291, K299, and K381 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K289, K291, K299, and K381 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K289, K291, K299, and K381 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 65. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 65. In some embodiments, the modified BoNT polypeptide comprises the amino acid sequence of any one of SEQ ID NOs: 187-194 having arginine substitutions at positions corresponding to one or more of K289, K291, K299, and K381 in SEQ ID NO: 1. In some embodiments, the modified BoNT polypeptide comprises the amino acid sequence of any one of SEQ ID NOs: 187-194 having arginine substitutions at positions corresponding to K289, K291, K299, and K381 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K212, K272, and K356 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K212, K272, and K356 in SEQ ID NO: 1. In some embodiments, the group of arginine substitutions at positions corresponding to K212, K272, and K356 in SEQ ID NO: 1 is referred to as Group 3. In some embodiments, the modified BoNT polypeptide comprises the amino acid sequence of SEQ ID NO: 109. In some embodiments, the modified BoNT polypeptide consists of the amino acid sequence of SEQ ID NO: 109.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K212, K272, and K356 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K212, K272, and K356 in SEQ ID NO: 1. In some embodiments, the modified BoNT polypeptide comprises the amino acid sequence of SEQ ID NO: 66. In some embodiments, the modified BoNT polypeptide consists of the amino acid sequence of SEQ ID NO: 66.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K212, K272, and K356 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K212, K272, and K356 in SEQ ID NO: 1. In some embodiments, the modified BoNT polypeptide comprises the amino acid sequence of SEQ ID NO: 23. In some embodiments, the modified BoNT polypeptide consists of the amino acid sequence of SEQ ID NO: 23.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K212, K272, and K356 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K212, K272, and K356 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K212, K272, and K356 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 66 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K212, K272, and K356 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K212, K272, and K356 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K212, K272, and K356 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K212, K272, and K356 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 66. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 66. In some embodiments, the modified BoNT polypeptide comprises the amino acid sequence of any one of SEQ ID NOs: 187-194 having arginine substitutions at positions corresponding to one or more of K212, K272, and K356 in SEQ ID NO: 1. In some embodiments, the modified BoNT polypeptide comprises the amino acid sequence of any one of SEQ ID NOs: 187-194 having arginine substitutions at positions corresponding to K212, K272, and K356 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K41, K318, K335, K337, K340, K343, K375, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K41, K318, K335, K337, K340, K343, K375, and K415 in SEQ ID NO: 1. In some embodiments, the group of arginine substitutions at positions corresponding to K11, K41, K318, K335, K337, K340, K343, K375, and K415 in SEQ ID NO: 1 is referred to as KR9. In some embodiments, the modified BoNT polypeptide comprises the amino acid sequence of SEQ ID NO: 110. In some embodiments, the modified BoNT polypeptide consists of the amino acid sequence of SEQ ID NO: 110.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K41, K318, K335, K337, K340, K343, K375, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K41, K318, K335, K337, K340, K343, K375, and K415 in SEQ ID NO: 1. In some embodiments, the modified BoNT polypeptide comprises the amino acid sequence of SEQ ID NO: 67. In some embodiments, the modified BoNT polypeptide consists of the amino acid sequence of SEQ ID NO: 67.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K41, K318, K335, K337, K340, K343, K375, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K41, K318, K335, K337, K340, K343, K375, and K415 in SEQ ID NO: 1. In some embodiments, the modified BoNT polypeptide comprises the amino acid sequence of SEQ ID NO: 24. In some embodiments, the modified BoNT polypeptide consists of the amino acid sequence of SEQ ID NO: 24.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K41, K318, K335, K337, K340, K343, K375, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K41, K318, K335, K337, K340, K343, K375, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K41, K318, K335, K337, K340, K343, K375, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 67 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K41, K318, K335, K337, K340, K343, K375, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K41, K318, K335, K337, K340, K343, K375, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K41, K318, K335, K337, K340, K343, K375, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K41, K318, K335, K337, K340, K343, K375, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 67. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 67. In some embodiments, the modified BoNT polypeptide comprises the amino acid sequence of any one of SEQ ID NOs: 187-194 having arginine substitutions at positions corresponding to one or more of K11, K41, K318, K335, K337, K340, K343, K375, and K415 in SEQ ID NO: 1. In some embodiments, the modified BoNT polypeptide comprises the amino acid sequence of any one of SEQ ID NOs: 187-194 having arginine substitutions at positions corresponding to K11, K41, K318, K335, K337, K340, K343, K375, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K289, K291, K299, K335, K337, K343, K375, K381, and K415 in SEQ ID NO: 1. In some embodiments, the group of arginine substitutions at positions corresponding to K11, K289, K291, K299, K335, K337, K343, K375, K381, and K415 in SEQ ID NO: 1 is referred to as KR10. In some embodiments, the modified BoNT polypeptide comprises the amino acid sequence of SEQ ID NO: 111. In some embodiments, the modified BoNT polypeptide consists of the amino acid sequence of SEQ ID NO: 111.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K289, K291, K299, K335, K337, K343, K375, K381, and K415 in SEQ ID NO: 1. In some embodiments, the modified BoNT polypeptide comprises the amino acid sequence of SEQ ID NO: 68. In some embodiments, the modified BoNT polypeptide consists of the amino acid sequence of SEQ ID NO: 68.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K289, K291, K299, K335, K337, K343, K375, K381, and K415 in SEQ ID NO: 1. In some embodiments, the modified BoNT polypeptide comprises the amino acid sequence of SEQ ID NO: 25. In some embodiments, the modified BoNT polypeptide consists of the amino acid sequence of SEQ ID NO: 25.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K289, K291, K299, K335, K337, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 68 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K289, K291, K299, K335, K337, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K289, K291, K299, K335, K337, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 68. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 68. In some embodiments, the modified BoNT polypeptide comprises the amino acid sequence of any one of SEQ ID NOs: 187-194 having arginine substitutions at positions corresponding to one or more of K11, K289, K291, K299, K335, K337, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K289, K291, K299, K335, K337, K343, K375, K381, and K415 in SEQ ID NO: 1.

In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to SEQ ID NO: 203 or SEQ ID NO: 217. In some embodiments, the modified BoNT polypeptide comprises a polypeptide comprising an amino acid sequence of SEQ ID NO: 203 or SEQ ID NO: 217. In some embodiments, the modified BoNT polypeptide comprises a polypeptide consisting of an amino acid sequence of SEQ ID NO: 203 or SEQ ID NO: 217.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K41, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K41, K289, K291, K299, K335, K337, K343, K375, K381, and K415 in SEQ ID NO: 1. In some embodiments, the group of arginine substitutions at positions corresponding to K11, K41, K289, K291, K299, K335, K337, K343, K375, K381, and K415 in SEQ ID NO: 1 is referred to as KR11-41. In some embodiments, the modified BoNT polypeptide comprises the amino acid sequence of SEQ ID NO: 112. In some embodiments, the modified BoNT polypeptide consists of the amino acid sequence of SEQ ID NO: 112.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K41, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K41, K289, K291, K299, K335, K337, K343, K375, K381, and K415 in SEQ ID NO: 1. In some embodiments, the modified BoNT polypeptide comprises the amino acid sequence of SEQ ID NO: 69. In some embodiments, the modified BoNT polypeptide consists of the amino acid sequence of SEQ ID NO: 69.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K41, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K41, K289, K291, K299, K335, K337, K343, K375, K381, and K415 in SEQ ID NO: 1. In some embodiments, the modified BoNT polypeptide comprises the amino acid sequence of SEQ ID NO: 26. In some embodiments, the modified BoNT polypeptide consists of the amino acid sequence of SEQ ID NO: 26.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K41, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K41, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K41, K289, K291, K299, K335, K337, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 69 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K41, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K41, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K41, K289, K291, K299, K335, K337, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K41, K289, K291, K299, K335, K337, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 69. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 69. In some embodiments, the modified BoNT polypeptide comprises the amino acid sequence of any one of SEQ ID NOs: 187-194 having arginine substitutions at positions corresponding to one or more of K11, K41, K289, K291, K299, K335, K337, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K41, K289, K291, K299, K335, K337, K343, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K212, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K212, K289, K291, K299, K335, K337, K343, K375, K381, and K415 in SEQ ID NO: 1. In some embodiments, the group of arginine substitutions at positions corresponding to K11, K212, K289, K291, K299, K335, K337, K343, K375, K381, and K415 in SEQ ID NO: 1 is referred to as KR11-212. In some embodiments, the modified BoNT polypeptide comprises the amino acid sequence of SEQ ID NO: 113. In some embodiments, the modified BoNT polypeptide consists of the amino acid sequence of SEQ ID NO: 113.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K212, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K212, K289, K291, K299, K335, K337, K343, K375, K381, and K415 in SEQ ID NO: 1. In some embodiments, the modified BoNT polypeptide comprises the amino acid sequence of SEQ ID NO: 70. In some embodiments, the modified BoNT polypeptide consists of the amino acid sequence of SEQ ID NO: 70.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K212, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K212, K289, K291, K299, K335, K337, K343, K375, K381, and K415 in SEQ ID NO: 1. In some embodiments, the modified BoNT polypeptide comprises the amino acid sequence of SEQ ID NO: 27. In some embodiments, the modified BoNT polypeptide consists of the amino acid sequence of SEQ ID NO: 27.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K212, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K212, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K212, K289, K291, K299, K335, K337, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 70 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K212, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K212, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K212, K289, K291, K299, K335, K337, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K212, K289, K291, K299, K335, K337, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 70. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 70. In some embodiments, the modified BoNT polypeptide comprises the amino acid sequence of any one of SEQ ID NOs: 187-194 having arginine substitutions at positions corresponding to one or more of K11, K212, K289, K291, K299, K335, K337, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K212, K289, K291, K299, K335, K337, K343, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K272, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K272, K289, K291, K299, K335, K337, K343, K375, K381, and K415 in SEQ ID NO: 1. In some embodiments, the group of arginine substitutions at positions corresponding to K11, K272, K289, K291, K299, K335, K337, K343, K375, K381, and K415 in SEQ ID NO: 1 is referred to as KR11-272. In some embodiments, the modified BoNT polypeptide comprises the amino acid sequence of SEQ ID NO: 114. In some embodiments, the modified BoNT polypeptide consists of the amino acid sequence of SEQ ID NO: 114.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K272, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K272, K289, K291, K299, K335, K337, K343, K375, K381, and K415 in SEQ ID NO: 1. In some embodiments, the modified BoNT polypeptide comprises the amino acid sequence of SEQ ID NO: 71. In some embodiments, the modified BoNT polypeptide consists of the amino acid sequence of SEQ ID NO: 71.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K272, K289, K291, K299, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K272, K289, K291, K299, K335, K337, K343, K375, K381, and K415 in SEQ ID NO: 1. In some embodiments, the modified BoNT polypeptide comprises the amino acid sequence of SEQ ID NO: 28. In some embodiments, the modified BoNT polypeptide consists of the amino acid sequence of SEQ ID NO: 28.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K272, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K272, K289, K291, K299, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K272, K289, K291, K299, K335, K337, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 71 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K272, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K272, K289, K291, K299, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K272, K289, K291, K299, K335, K337, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K272, K289, K291, K299, K335, K337, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 71. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 71. In some embodiments, the modified BoNT polypeptide comprises the amino acid sequence of any one of SEQ ID NOs: 187-194 having arginine substitutions at positions corresponding to one or more of K11, K272, K289, K291, K299, K335, K337, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K272, K289, K291, K299, K335, K337, K343, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K289, K291, K299, K318, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K289, K291, K299, K318, K335, K337, K343, K375, K381, and K415 in SEQ ID NO: 1. In some embodiments, the group of arginine substitutions at positions corresponding to K11, K289, K291, K299, K318, K335, K337, K343, K375, K381, and K415 in SEQ ID NO: 1 is referred to as KR11-318. In some embodiments, the modified BoNT polypeptide comprises the amino acid sequence of SEQ ID NO: 115. In some embodiments, the modified BoNT polypeptide consists of the amino acid sequence of SEQ ID NO: 115.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K289, K291, K299, K318, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K289, K291, K299, K318, K335, K337, K343, K375, K381, and K415 in SEQ ID NO: 1. In some embodiments, the modified BoNT polypeptide comprises the amino acid sequence of SEQ ID NO: 72. In some embodiments, the modified BoNT polypeptide consists of the amino acid sequence of SEQ ID NO: 72.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K289, K291, K299, K318, K335, K337, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K289, K291, K299, K318, K335, K337, K343, K375, K381, and K415 in SEQ ID NO: 1. In some embodiments, the modified BoNT polypeptide comprises the amino acid sequence of SEQ ID NO: 29. In some embodiments, the modified BoNT polypeptide consists of the amino acid sequence of SEQ ID NO: 29.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K289, K291, K299, K318, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K289, K291, K299, K318, K335, K337, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K289, K291, K299, K318, K335, K337, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 72 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K289, K291, K299, K318, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K289, K291, K299, K318, K335, K337, K343, K375, K381, and K415 in SEQ ID NO: 1. In some embodiments, the modified BoNT polypeptide comprises a polypeptide comprising an amino acid sequence that is at least 70% (e.g., at least 70%, at identical to any one of SEQ ID NOs: 179-186, fused to a polypeptide comprising the translocation domain of BoNT/A1 and a modified protease domain of BoNT/A1 comprising arginine substitutions at positions corresponding to K11, K289, K291, K299, K318, K335, K337, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K289, K291, K299, K318, K335, K337, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 72. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 72. In some embodiments, the modified BoNT polypeptide comprises the amino acid sequence of any one of SEQ ID NOs: 187-194 having arginine substitutions at positions corresponding to one or more of K11, K289, K291, K299, K318, K335, K337, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K289, K291, K299, K318, K335, K337, K343, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K289, K291, K299, K335, K337, K340, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K289, K291, K299, K335, K337, K340, K343, K375, K381, and K415 in SEQ ID NO: 1. In some embodiments, the group of arginine substitutions at positions corresponding to K11, K289, K291, K299, K335, K337, K340, K343, K375, K381, and K415 in SEQ ID NO: 1 is referred to as KR11-340. In some embodiments, the modified BoNT polypeptide comprises the amino acid sequence of SEQ ID NO: 116. In some embodiments, the modified BoNT polypeptide consists of the amino acid sequence of SEQ ID NO: 116.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K289, K291, K299, K335, K337, K340, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K289, K291, K299, K335, K337, K340, K343, K375, K381, and K415 in SEQ ID NO: 1. In some embodiments, the modified BoNT polypeptide comprises the amino acid sequence of SEQ ID NO: 73. In some embodiments, the modified BoNT polypeptide consists of the amino acid sequence of SEQ ID NO: 73.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K289, K291, K299, K335, K337, K340, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K289, K291, K299, K335, K337, K340, K343, K375, K381, and K415 in SEQ ID NO: 1. In some embodiments, the modified BoNT polypeptide comprises the amino acid sequence of SEQ ID NO: 30. In some embodiments, the modified BoNT polypeptide consists of the amino acid sequence of SEQ ID NO: 30.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K289, K291, K299, K335, K337, K340, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K289, K291, K299, K335, K337, K340, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K289, K291, K299, K335, K337, K340, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 73 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K289, K291, K299, K335, K337, K340, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K289, K291, K299, K335, K337, K340, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K289, K291, K299, K335, K337, K340, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K289, K291, K299, K335, K337, K340, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 73. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 73. In some embodiments, the modified BoNT polypeptide comprises the amino acid sequence of any one of SEQ ID NOs: 187-194 having arginine substitutions at positions corresponding to one or more of K11, K289, K291, K299, K335, K337, K340, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K289, K291, K299, K335, K337, K340, K343, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K289, K291, K299, K335, K337, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K289, K291, K299, K335, K337, K343, K356, K375, K381, and K415 in SEQ ID NO: 1. In some embodiments, the group of arginine substitutions at positions corresponding to K11, K289, K291, K299, K335, K337, K343, K356, K375, K381, and K415 in SEQ ID NO: 1 is referred to as KR11-356. In some embodiments, the modified BoNT polypeptide comprises the amino acid sequence of SEQ ID NO: 117. In some embodiments, the modified BoNT polypeptide consists of the amino acid sequence of SEQ ID NO: 117.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K289, K291, K299, K335, K337, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K289, K291, K299, K335, K337, K343, K356, K375, K381, and K415 in SEQ ID NO: 1. In some embodiments, the modified BoNT polypeptide comprises the amino acid sequence of SEQ ID NO: 74. In some embodiments, the modified BoNT polypeptide consists of the amino acid sequence of SEQ ID NO: 74.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K289, K291, K299, K335, K337, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K289, K291, K299, K335, K337, K343, K356, K375, K381, and K415 in SEQ ID NO: 1. In some embodiments, the modified BoNT polypeptide comprises the amino acid sequence of SEQ ID NO: 31. In some embodiments, the modified BoNT polypeptide consists of the amino acid sequence of SEQ ID NO: 31.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K289, K291, K299, K335, K337, K343, K356, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K289, K291, K299, K335, K337, K343, K356, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K289, K291, K299, K335, K337, K343, K356, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 74 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K289, K291, K299, K335, K337, K343, K356, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K289, K291, K299, K335, K337, K343, K356, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K289, K291, K299, K335, K337, K343, K356, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K289, K291, K299, K335, K337, K343, K356, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 74. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 74. In some embodiments, the modified BoNT polypeptide comprises the amino acid sequence of any one of SEQ ID NOs: 187-194 having arginine substitutions at positions corresponding to one or more of K11, K289, K291, K299, K335, K337, K343, K356, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K289, K291, K299, K335, K337, K343, K356, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K41, K289, K291, K299, K318, K335, K337, K340, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K41, K289, K291, K299, K318, K335, K337, K340, K343, K375, K381, and K415 in SEQ ID NO: 1. In some embodiments, the group of arginine substitutions at positions corresponding to K11, K41, K289, K291, K299, K318, K335, K337, K340, K343, K375, K381, and K415 in SEQ ID NO: 1 is referred to as KR13. In some embodiments, the modified BoNT polypeptide comprises the amino acid sequence of SEQ ID NO: 118. In some embodiments, the modified BoNT polypeptide consists of the amino acid sequence of SEQ ID NO: 118.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K41, K289, K291, K299, K318, K335, K337, K340, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K41, K289, K291, K299, K318, K335, K337, K340, K343, K375, K381, and K415 in SEQ ID NO: 1. In some embodiments, the modified BoNT polypeptide comprises the amino acid sequence of SEQ ID NO: 75. In some embodiments, the modified BoNT polypeptide consists of the amino acid sequence of SEQ ID NO: 75.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K41, K289, K291, K299, K318, K335, K337, K340, K343, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K41, K289, K291, K299, K318, K335, K337, K340, K343, K375, K381, and K415 in SEQ ID NO: 1. In some embodiments, the modified BoNT polypeptide comprises the amino acid sequence of SEQ ID NO: 32. In some embodiments, the modified BoNT polypeptide consists of the amino acid sequence of SEQ ID NO: 32.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K41, K289, K291, K299, K318, K335, K337, K340, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K41, K289, K291, K299, K318, K335, K337, K340, K343, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K41, K289, K291, K299, K318, K335, K337, K340, K343, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 75 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K41, K289, K291, K299, K318, K335, K337, K340, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K41, K289, K291, K299, K318, K335, K337, K340, K343, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K41, K289, K291, K299, K318, K335, K337, K340, K343, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K41, K289, K291, K299, K318, K335, K337, K340, K343, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 75. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 75. In some embodiments, the modified BoNT polypeptide comprises the amino acid sequence of any one of SEQ ID NOs: 187-194 having arginine substitutions at positions corresponding to one or more of K11, K41, K289, K291, K299, K318, K335, K337, K340, K343, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K41, K289, K291, K299, K318, K335, K337, K340, K343, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K41, K212, K289, K291, K299, K318, K335, K337, K340, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K41, K212, K289, K291, K299, K318, K335, K337, K340, K343, K356, K375, K381, and K415 in SEQ ID NO: 1. In some embodiments, the group of arginine substitutions at positions corresponding to K11, K41, K212, K289, K291, K299, K318, K335, K337, K340, K343, K356, K375, K381, and K415 in SEQ ID NO: 1 is referred to as KR15. In some embodiments, the modified BoNT polypeptide comprises the amino acid sequence of SEQ ID NO: 119. In some embodiments, the modified BoNT polypeptide consists of the amino acid sequence of SEQ ID NO: 119.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K41, K212, K289, K291, K299, K318, K335, K337, K340, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K41, K212, K289, K291, K299, K318, K335, K337, K340, K343, K356, K375, K381, and K415 in SEQ ID NO: 1. In some embodiments, the modified BoNT polypeptide comprises the amino acid sequence of SEQ ID NO: 76. In some embodiments, the modified BoNT polypeptide consists of the amino acid sequence of SEQ ID NO: 76.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K41, K212, K289, K291, K299, K318, K335, K337, K340, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K41, K212, K289, K291, K299, K318, K335, K337, K340, K343, K356, K375, K381, and K415 in SEQ ID NO: 1. In some embodiments, the modified BoNT polypeptide comprises the amino acid sequence of SEQ ID NO: 33. In some embodiments, the modified BoNT polypeptide consists of the amino acid sequence of SEQ ID NO: 33.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K41, K212, K289, K291, K299, K318, K335, K337, K340, K343, K356, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K41, K212, K289, K291, K299, K318, K335, K337, K340, K343, K356, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K41, K212, K289, K291, K299, K318, K335, K337, K340, K343, K356, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 76 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K41, K212, K289, K291, K299, K318, K335, K337, K340, K343, K356, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K41, K212, K289, K291, K299, K318, K335, K337, K340, K343, K356, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K41, K212, K289, K291, K299, K318, K335, K337, K340, K343, K356, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K41, K212, K289, K291, K299, K318, K335, K337, K340, K343, K356, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 76. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 76. In some embodiments, the modified BoNT polypeptide comprises the amino acid sequence of any one of SEQ ID NOs: 187-194 having arginine substitutions at positions corresponding to one or more of K11, K41, K212, K289, K291, K299, K318, K335, K337, K340, K343, K356, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K41, K212, K289, K291, K299, K318, K335, K337, K340, K343, K356, K375, K381, and K415 in SEQ ID NO: 1.

In some embodiments, the modified BoNT polypeptide is a modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to one or more of K11, K41, K212, K272, K289, K291, K299, K318, K335, K337, K340, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions at positions corresponding to K11, K41, K212, K272, K289, K291, K299, K318, K335, K337, K340, K343, K356, K375, K381, and K415 in SEQ ID NO: 1. In some embodiments, the group of arginine substitutions at positions corresponding to K11, K41, K212, K272, K289, K291, K299, K318, K335, K337, K340, K343, K356, K375, K381, and K415 in SEQ ID NO: 1 is referred to as KR16. In some embodiments, the modified BoNT polypeptide comprises the amino acid sequence of SEQ ID NO: 120. In some embodiments, the modified BoNT polypeptide consists of the amino acid sequence of SEQ ID NO: 120.

In some embodiments, the modified BoNT polypeptide comprises a modified protease domain of BoNT/A1 and a translocation domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to one or more of K11, K41, K212, K272, K289, K291, K299, K318, K335, K337, K340, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO:1, and comprises arginine substitutions a positions corresponding to K11, K41, K212, K272, K289, K291, K299, K318, K335, K337, K340, K343, K356, K375, K381, and K415 in SEQ ID NO: 1. In some embodiments, the modified BoNT polypeptide comprises the amino acid sequence of SEQ ID NO: 77. In some embodiments, the modified BoNT polypeptide consists of the amino acid sequence of SEQ ID NO: 77.

In some embodiments, the modified BoNT polypeptide is a full-length BoNT/A1 polypeptide comprising the modified protease domain of BoNT/A1 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to one or more of K11, K41, K212, K272, K289, K291, K299, K318, K335, K337, K340, K343, K356, K375, K381, and K415 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions at positions corresponding to K11, K41, K212, K272, K289, K291, K299, K318, K335, K337, K340, K343, K356, K375, K381, and K415 in SEQ ID NO: 1. In some embodiments, the modified BoNT polypeptide comprises the amino acid sequence of SEQ ID NO: 34. In some embodiments, the modified BoNT polypeptide consists of the amino acid sequence of SEQ ID NO: 34.

In some embodiments, the modified BoNT polypeptide comprises a modified protease domain of BoNT/A1, a translocation domain of BoNT/A1, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to one or more of K11, K41, K212, K272, K289, K291, K299, K318, K335, K337, K340, K343, K356, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 1, and comprises arginine substitutions a positions corresponding to K11, K41, K212, K272, K289, K291, K299, K318, K335, K337, K340, K343, K356, K375, K381, and K415 in SEQ ID NO: 1 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1. In some embodiments, the receptor binding domain comprises an amino acid substitution at a position corresponding to 1156 or 1232 in SEQ ID NO: 1. In some embodiments, the receptor binding domain comprises an amino acid substitution that corresponds to R1156M or T1232R in SEQ ID NO: 1.

In some embodiments, the modified BoNT polypeptide comprises a modified protease domain of BoNT/A1, a translocation domain of BoNT/A1, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 78 fused to the amino acid sequence of any one of SEQ ID NO: 131-165 or 205, and comprises arginine substitutions at positions corresponding to one or more of K11, K41, K212, K272, K289, K291, K299, K318, K335, K337, K340, K343, K356, K375, K381, and K415 in SEQ ID NO: 1 and one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 77 fused to the amino acid sequence of any one of SEQ ID NO: 131-165 or 205, and comprising one or more amino acid substitutions in the receptor binding domain at positions corresponding to 915, 923, 1090, 1103, 1117, 1156, 1170, 1227, 1254, 1255, or 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A1 and a modified protease domain of BoNT/A1 comprising arginine substitutions at positions corresponding to K11, K41, K212, K272, K289, K291, K299, K318, K335, K337, K340, K343, K356, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to one or more of K11, K41, K212, K272, K289, K291, K299, K318, K335, K337, K340, K343, K356, K375, K381, and K415 in SEQ ID NO: 1. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A1 and a modified protease domain of BoNT/A1 comprising arginine substitutions at positions corresponding to K11, K41, K212, K272, K289, K291, K299, K318, K335, K337, K340, K343, K356, K375, K381, and K415 in SEQ ID NO: 1. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 44 and comprising arginine substitutions at positions corresponding to K11, K41, K212, K272, K289, K291, K299, K318, K335, K337, K340, K343, K356, K375, K381, and K415 in SEQ ID NO: 1. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 77. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 77. In some embodiments, the modified BoNT polypeptide comprises the amino acid sequence of any one of SEQ ID NOs: 187-194 having arginine substitutions at positions corresponding to one or more of K11, K41, K212, K272, K289, K291, K299, K318, K335, K337, K340, K343, K356, K375, K381, and K415 in SEQ ID NO: 1. In some embodiments, the modified BoNT polypeptide comprises the amino acid sequence of any one of SEQ ID NOs: 187-194 having arginine substitutions at positions corresponding to K11, K41, K212, K272, K289, K291, K299, K318, K335, K337, K340, K343, K356, K375, K381, and K415 in SEQ ID NO: 1.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K11 in SEQ ID NO: 35. In some embodiments, the modified BoNT polypeptide comprises the amino acid sequence of SEQ ID NO: 122. In some embodiments, the modified BoNT polypeptide consists of the amino acid sequence of SEQ ID NO: 122.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K11 in SEQ ID NO: 35. In some embodiments, the modified BoNT polypeptide comprises the amino acid sequence of SEQ ID NO: 79. In some embodiments, the modified BoNT polypeptide consists of the amino acid sequence of SEQ ID NO: 79.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K11 in SEQ ID NO: 35. In some embodiments, the modified BoNT polypeptide comprises the amino acid sequence of SEQ ID NO: 36. In some embodiments, the modified BoNT polypeptide consists of the amino acid sequence of SEQ ID NO: 36.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least to 85%, at least 90%, at least 95%, or at least 99%) identical to SEQ ID NO: 35, and comprises an arginine substitution corresponding to K11 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises an arginine substitution corresponding to K11 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 79 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K11 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K11 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K11 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 79. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 79. In some embodiments, the modified BoNT polypeptide comprises the amino acid sequence of any one of SEQ ID NOs: 187-194 having an arginine corresponding to K11 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K289 in SEQ ID NO: 35. In some embodiments, the modified BoNT polypeptide comprises the amino acid sequence of SEQ ID NO: 123. In some embodiments, the modified BoNT polypeptide consists of the amino acid sequence of SEQ ID NO: 123.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K289 in SEQ ID NO: 35. In some embodiments, the modified BoNT polypeptide comprises the amino acid sequence of SEQ ID NO: 80. In some embodiments, the modified BoNT polypeptide consists of the amino acid sequence of SEQ ID NO: 80.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K289 in SEQ ID NO: 35. In some embodiments, the modified BoNT polypeptide comprises the amino acid sequence of SEQ ID NO: 37. In some embodiments, the modified BoNT polypeptide consists of the amino acid sequence of SEQ ID NO: 37.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K289 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises an arginine substitution corresponding to K289 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 80 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K289 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K289 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K289 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 80. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 80. In some embodiments, the modified BoNT polypeptide comprises the amino acid sequence of any one of SEQ ID NOs: 187-194 having an arginine corresponding to K289 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K291 in SEQ ID NO: 35. In some embodiments, the modified BoNT polypeptide comprises the amino acid sequence of SEQ ID NO: 124. In some embodiments, the modified BoNT polypeptide consists of the amino acid sequence of SEQ ID NO: 124.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K291 in SEQ ID NO: 35. In some embodiments, the modified BoNT polypeptide comprises the amino acid sequence of SEQ ID NO: 81. In some embodiments, the modified BoNT polypeptide consists of the amino acid sequence of SEQ ID NO: 81.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K291 in SEQ ID NO: 35. In some embodiments, the modified BoNT polypeptide comprises the amino acid sequence of SEQ ID NO: 38. In some embodiments, the modified BoNT polypeptide consists of the amino acid sequence of SEQ ID NO: 38.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K291 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, S1090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises an arginine substitution corresponding to K291 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 81 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K291 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K291 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K291 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 81. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 81. In some embodiments, the modified BoNT polypeptide comprises the amino acid sequence of any one of SEQ ID NOs: 187-194 having an arginine corresponding to K291 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K299 in SEQ ID NO: 35. In some embodiments, the modified BoNT polypeptide comprises the amino acid sequence of SEQ ID NO: 125. In some embodiments, the modified BoNT polypeptide consists of the amino acid sequence of SEQ ID NO: 125.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K299 in SEQ ID NO: 35. In some embodiments, the modified BoNT polypeptide comprises the amino acid sequence of SEQ ID NO: 82. In some embodiments, the modified BoNT polypeptide consists of the amino acid sequence of SEQ ID NO: 82.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K299 in SEQ ID NO: 35. In some embodiments, the modified BoNT polypeptide comprises the amino acid sequence of SEQ ID NO: 39. In some embodiments, the modified BoNT polypeptide consists of the amino acid sequence of SEQ ID NO: 39.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K299 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises an arginine substitution corresponding to K299 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 82 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K299 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K299 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K299 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 82. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 82. In some embodiments, the modified BoNT polypeptide comprises the amino acid sequence of any one of SEQ ID NOs: 187-194 having an arginine corresponding to K299 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K335 in SEQ ID NO: 35. In some embodiments, the modified BoNT polypeptide comprises the amino acid sequence of SEQ ID NO: 126. In some embodiments, the modified BoNT polypeptide consists of the amino acid sequence of SEQ ID NO: 126.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K335 in SEQ ID NO: 35. In some embodiments, the modified BoNT polypeptide comprises the amino acid sequence of SEQ ID NO: 83. In some embodiments, the modified BoNT polypeptide consists of the amino acid sequence of SEQ ID NO: 83.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K335 in SEQ ID NO: 35. In some embodiments, the modified BoNT polypeptide comprises the amino acid sequence of SEQ ID NO: 40. In some embodiments, the modified BoNT polypeptide consists of the amino acid sequence of SEQ ID NO: 40.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K335 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at identical to SEQ ID NO: 44 fused to the amino acid sequence of any one of SEQ ID NO: 166-176 or 204, and comprises an arginine substitution corresponding to K335 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 83 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D 1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K335 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K335 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K335 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 83. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 83. In some embodiments, the modified BoNT polypeptide comprises the amino acid sequence of any one of SEQ ID NOs: 187-194 having an arginine corresponding to K335 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K337 in SEQ ID NO: 35. In some embodiments, the modified BoNT polypeptide comprises the amino acid sequence of SEQ ID NO: 127. In some embodiments, the modified BoNT polypeptide consists of the amino acid sequence of SEQ ID NO: 127.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K337 in SEQ ID NO: 35. In some embodiments, the modified BoNT polypeptide comprises the amino acid sequence of SEQ ID NO: 84. In some embodiments, the modified BoNT polypeptide consists of the amino acid sequence of SEQ ID NO: 84.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K337 in SEQ ID NO: 35. In some embodiments, the modified BoNT polypeptide comprises the amino acid sequence of SEQ ID NO: 41. In some embodiments, the modified BoNT polypeptide consists of the amino acid sequence of SEQ ID NO: 41.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at identical to SEQ ID NO: 35, and comprises an arginine substitution corresponding to K337 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises an arginine substitution corresponding to K337 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 84 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K337 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K337 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K337 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 84. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 84. In some embodiments, the modified BoNT polypeptide comprises the amino acid sequence of any one of SEQ ID NOs: 187-194 having an arginine corresponding to K337 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K343 in SEQ ID NO: 35. In some embodiments, the modified BoNT polypeptide comprises the amino acid sequence of SEQ ID NO: 128. In some embodiments, the modified BoNT polypeptide consists of the amino acid sequence of SEQ ID NO: 128.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K343 in SEQ ID NO: 35. In some embodiments, the modified BoNT polypeptide comprises the amino acid sequence of SEQ ID NO: 85. In some embodiments, the modified BoNT polypeptide consists of the amino acid sequence of SEQ ID NO: 85.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K343 in SEQ ID NO: 35. In some embodiments, the modified BoNT polypeptide comprises the amino acid sequence of SEQ ID NO: 42. In some embodiments, the modified BoNT polypeptide consists of the amino acid sequence of SEQ ID NO: 42.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises an arginine substitution corresponding to K343 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises an arginine substitution corresponding to K343 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that comprises SEQ ID NO: 85 fused to the amino acid sequence of any one of SEQ ID NO: 166-176 or 204, and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1 in SEQ ID NO: 1.

In some embodiments, the modified BoNT polypeptide is a chimeric BoNT a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising an arginine substitution corresponding to K343 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising an arginine substitution corresponding to K343 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprises an arginine substitution corresponding to K343 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 85. In some embodiments, the modified BoNT polypeptide comprises a polypeptide consisting of the amino acid sequence of any one of SEQ ID NOs: 179-186, fused to a polypeptide consisting of the amino acid sequence of SEQ ID NO: 85. In some embodiments, the modified BoNT polypeptide comprises the amino acid sequence of any one of SEQ ID NOs: 187-194 having an arginine corresponding to K343 in SEQ ID NO: 35.

In some embodiments, the modified BoNT polypeptide is a modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions at positions corresponding to one or more of K11, K289, K291, K299, K335, K337, and K343 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions at positions corresponding to K11, K289, K291, K299, K335, K337, and K343 in SEQ ID NO: 35. In some embodiments, the group of arginine substitutions at positions corresponding to K11, K289, K291, K299, K335, K337, and K343 in SEQ ID NO: 35 is referred to as KR7. In some embodiments, the modified BoNT polypeptide comprises the amino acid sequence of SEQ ID NO: 129. In some embodiments, the modified BoNT polypeptide consists of the amino acid sequence of SEQ ID NO: 129.

In some embodiments, the modified BoNT polypeptide comprises a modified protease domain of BoNT/A2 and a translocation domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions a positions corresponding to one or more of K11, K289, K291, K299, K335, K337, and K343 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions a positions corresponding to K11, K289, K291, K299, K335, K337, and K343 in SEQ ID NO: 35. In some embodiments, the modified BoNT polypeptide comprises the amino acid sequence of SEQ ID NO: 86. In some embodiments, the modified BoNT polypeptide consists of the amino acid sequence of SEQ ID NO: 86.

In some embodiments, the modified BoNT polypeptide is a full-length BoNT/A2 polypeptide comprising the modified protease domain of BoNT/A2 described herein. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions at positions corresponding to one or more of K11, K289, K291, K299, K335, K337, and K343 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions at positions corresponding to K11, K289, K291, K299, K335, K337, and K343 in SEQ ID NO: 35. In some embodiments, the modified BoNT polypeptide comprises the amino acid sequence of SEQ ID NO: 43. In some embodiments, the modified BoNT polypeptide consists of the amino acid sequence of SEQ ID NO: 43.

In some embodiments, the modified BoNT polypeptide comprises a modified protease domain of BoNT/A2, a translocation domain of BoNT/A2, and a modified receptor binding domain of BoNT/A2. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions a positions corresponding to one or more of K11, K289, K291, K299, K335, K337, and K343 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 35, and comprises arginine substitutions a positions corresponding to K11, K289, K291, K299, K335, K337, and K343 in SEQ ID NO: 35 and one or more amino acid substitutions in the receptor binding domain at positions corresponding to 915, 923, 1090, 1103, 1117, 1156, 1170, 1227, 1254, 1255, and 1256 in SEQ ID NO: 35. In some embodiments the one or more amino acid substitutions in the receptor binding domain correspond to one or more of K915Q, T923K, 51090N, N1103D, F1117Y, E1156M, E1170K, D1227N, L1254Q, Y1255G, or D1256N in SEQ ID NO: 35.

In some embodiments, the modified BoNT polypeptide comprises a modified protease domain of BoNT/A2, a translocation domain of BoNT/A2, and a modified receptor binding domain of BoNT/A1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 44 fused to the amino acid sequence of any one of SEQ ID NO: 166-176 or 204, and comprises arginine substitutions at positions corresponding to one or more of K11, K289, K291, K299, K335, K337, and K343 in SEQ ID NO: 35 and one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified BoNT polypeptide comprises an amino acid sequence that is at least 70% (e.g., at least 70%, at least 75%, at least 80%, at least 85%, at least 90%, at least 95%, or at least 99%) identical to SEQ ID NO: 86 fused to the amino acid sequence of any one of SEQ ID NO: 166-176 or 204, and comprising one or more amino acid substitutions in the receptor binding domain at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments the one or more amino acid substitutions in the receptor binding domain correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1.

In some embodiments, the modified BoNT polypeptide is a chimeric BoNT comprising a receptor binding domain from a BoNT of a different serotype (e.g., BoNT/B, C, D, E, F, G, H, or EN) fused to a polypeptide comprising the translocation domain of BoNT/A2 and a modified protease domain of BoNT/A2 comprising arginine substitutions at positions corresponding to K11, K289, K291, K299, K335, K337, and K343 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising arginine substitutions at positions corresponding to one or more of K11, K289, K291, K299, K335, K337, and K343 in SEQ ID NO: 35. In some embodiments, the modified BoNT polypeptide comprises a polypeptide comprising an amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising the translocation domain of BoNT/A2 and a modified protease domain of BoNT/A2 comprising arginine substitutions at positions corresponding to K11, K289, K291, K299, K335, K337, and K343 in SEQ ID NO: 35. In some embodiments, the modified BoNT polypeptide comprises a polypeptide comprising a receptor binding domain amino acid sequence that is at least 70% (e.g., at least 70%, at least 75%, at least 80%, at least 85%, at least 90%, at least 95%, or at least 99%) identical to any one of SEQ ID NOs: 179-186, fused to a polypeptide comprising an amino acid sequence that is at least 70% (e.g., at least 70%, at least 75%, at least 80%, at least 85%, at least 90%, at least 95%, or at least 99%) identical to SEQ ID NO: 78 and comprising arginine substitutions at positions corresponding to K11, K289, K291, K299, K335, K337, and K343 in SEQ ID NO: 35. In some embodiments, the modified BoNT polypeptide comprises a polypeptide comprising the amino acid sequence of any one of SEQ ID NOs: 179-186, fused to a polypeptide comprising the amino acid sequence of SEQ ID NO: 86. In some embodiments, the modified BoNT polypeptide comprises a polypeptide consisting of the amino acid sequence of any one of SEQ ID NOs: 179-186, fused to an amino acid sequence consisting of the amino acid sequence of SEQ ID NO: 86. In some embodiments, the modified BoNT polypeptide comprises the amino acid sequence of any one of SEQ ID NOs: 187-194 having arginine substitutions at positions corresponding to one or more of K11, K289, K291, K299, K335, K337, and K343 in SEQ ID NO: 35. In some embodiments, the modified BoNT polypeptide comprises the amino acid sequence of any one of SEQ ID NOs: 187-194 having arginine substitutions at positions corresponding to K11, K289, K291, K299, K335, K337, and K343 in SEQ ID NO: 35.

Some aspects of the present disclosure provide modified Clostridial Botulinum neurotoxin (BoNT) polypeptides comprising a modified protease domain and a modified receptor binding domain of Clostridial Botulinum serotype A1 (BoNT/A1). In some embodiments, the modified protease domain comprises any one of the modified protease domains of BoNT/A1 described herein and the modified receptor binding domain of BoNT/A1 comprises one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1. In some embodiments, the modified receptor binding domain of BoNT/A1 comprises one or more amino acid substitutions at positions corresponding to 954, 955, 957, 1063, 1064, 1025, 1026, 1156, 1232, 1278, 1294, and 1295 in SEQ ID NO: 1.

In some embodiments, the modified BoNT comprising a modified protease domain further comprises a modified receptor binding domain comprising an amino acid substitution at a position corresponding to 917 in SEQ ID NO: 1. In some embodiments, the modified receptor binding domain comprises an amino acid substitution corresponding to F917R or F917K in SEQ ID NO: 1.

In some embodiments, the modified BoNT comprising a modified protease domain further comprises a modified receptor binding domain comprising an amino acid substitution at a position corresponding to 953 in SEQ ID NO: 1. In some embodiments, the modified receptor binding domain comprises an amino acid substitution corresponding to F953H or F953Y in SEQ ID NO: 1.

In some embodiments, the modified BoNT comprising a modified protease domain further comprises a modified receptor binding domain comprising an amino acid substitution at a position corresponding to 954 in SEQ ID NO: 1. In some embodiments, the modified receptor binding domain comprises an amino acid substitution corresponding to N954S in SEQ ID NO: 1.

In some embodiments, the modified BoNT comprising a modified protease domain further comprises a modified receptor binding domain comprising an amino acid substitution at a position corresponding to 955 in SEQ ID NO: 1. In some embodiments, the modified receptor binding domain comprises an amino acid substitution corresponding to S955K in SEQ ID NO: 1.

In some embodiments, the modified BoNT comprising a modified protease domain further comprises a modified receptor binding domain comprising an amino acid substitution at a position corresponding to 957 in SEQ ID NO: 1. In some embodiments, the modified receptor binding domain comprises an amino acid substitution corresponding to S957N in SEQ ID NO: 1.

In some embodiments, the modified BoNT comprising a modified protease domain further comprises a modified receptor binding domain comprising an amino acid substitution at a position corresponding to 968 in SEQ ID NO: 1. In some embodiments, the modified receptor binding domain comprises an amino acid substitution corresponding to M968I in SEQ ID NO: 1.

In some embodiments, the modified BoNT comprising a modified protease domain further comprises a modified receptor binding domain comprising an amino acid substitution at a position corresponding to 1025 in SEQ ID NO: 1. In some embodiments, the modified receptor binding domain comprises an amino acid substitution corresponding to N1025T in SEQ ID NO: 1.

In some embodiments, the modified BoNT comprising a modified protease domain further comprises a modified receptor binding domain comprising an amino acid substitution at a position corresponding to 1026 in SEQ ID NO: 1. In some embodiments, the modified receptor binding domain comprises an amino acid substitution corresponding to N1026K in SEQ ID NO: 1.

In some embodiments, the modified BoNT comprising a modified protease domain further comprises a modified receptor binding domain comprising an amino acid substitution at a position corresponding to 1052 in SEQ ID NO: 1. In some embodiments, the modified receptor binding domain comprises an amino acid substitution corresponding to N1052K in SEQ ID NO: 1.

In some embodiments, the modified BoNT comprising a modified protease domain further comprises a modified receptor binding domain comprising an amino acid substitution at a position corresponding to 1062 in SEQ ID NO: 1. In some embodiments, the modified receptor binding domain comprises an amino acid substitution corresponding to D1062E in SEQ ID NO: 1.

In some embodiments, the modified BoNT comprising a modified protease domain further comprises a modified receptor binding domain comprising an amino acid substitution at a position corresponding to 1063 in SEQ ID NO: 1. In some embodiments, the modified receptor binding domain comprises an amino acid substitution corresponding to T1063P in SEQ ID NO: 1.

In some embodiments, the modified BoNT comprising a modified protease domain further comprises a modified receptor binding domain comprising an amino acid substitution at a position corresponding to 1064 in SEQ ID NO: 1. In some embodiments, the modified receptor binding domain comprises an amino acid substitution corresponding to H1064R or H1064Q in SEQ ID NO: 1.

In some embodiments, the modified BoNT comprising a modified protease domain further comprises a modified receptor binding domain comprising an amino acid substitution at a position corresponding to 1065 in SEQ ID NO: 1. In some embodiments, the modified receptor binding domain comprises an amino acid substitution corresponding to R1065N in SEQ ID NO: 1. These modified receptor binding domains can be referred to as BoNT/A1-R1065* (any amino acid substitution at position corresponding to 1064) and BoNT/A1-R1065N.

In some embodiments, the modified BoNT comprising a modified protease domain further comprises a modified receptor binding domain comprising an amino acid substitution at a position corresponding to 1066 in SEQ ID NO: 1. In some embodiments, the modified receptor binding domain comprises an amino acid substitution corresponding to Y1066R or Y1066K in SEQ ID NO: 1.

In some embodiments, the modified BoNT comprising a modified protease domain further comprises a modified receptor binding domain comprising an amino acid substitution at a position corresponding to 1145 in SEQ ID NO: 1. In some embodiments, the modified receptor binding domain comprises an amino acid substitution corresponding to T1145Y in SEQ ID NO: 1.

In some embodiments, the modified BoNT comprising a modified protease domain further comprises a modified receptor binding domain comprising an amino acid substitution at a position corresponding to 1232 in SEQ ID NO: 1. In some embodiments, the modified receptor binding domain comprises an amino acid substitution corresponding to T1232R or T1232K in SEQ ID NO: 1. In some embodiments, the modified BoNT comprising a modified protease domain further comprises a modified receptor binding domain comprises SEQ ID NO: 178 or SEQ ID NO: 219. In some embodiments, the modified BoNT comprising a modified protease domain further comprises a modified receptor binding domain consists of SEQ ID NO: 178 or SEQ ID NO: 219.

In some embodiments, the modified BoNT comprising a modified protease domain further comprises a modified receptor binding domain comprising an amino acid substitution at a position corresponding to 1272 in SEQ ID NO: 1. In some embodiments, the modified receptor binding domain comprises an amino acid substitution corresponding to E1272G in SEQ ID NO: 1.

In some embodiments, the modified BoNT comprising a modified protease domain further comprises a modified receptor binding domain comprising an amino acid substitution at a position corresponding to 1278 in SEQ ID NO: 1. In some embodiments, the modified receptor binding domain comprises an amino acid substitution corresponding to L1278F, L1278Y or L1278W in SEQ ID NO: 1.

In some embodiments, the modified BoNT comprising a modified protease domain further comprises a modified receptor binding domain comprising an amino acid substitution at a position corresponding to 1288 in SEQ ID NO: 1. In some embodiments, the modified receptor binding domain comprises an amino acid substitution corresponding to D1288E in SEQ ID NO: 1.

In some embodiments, the modified BoNT comprising a modified protease domain further comprises a modified receptor binding domain comprising an amino acid substitution at a position corresponding to 1289 in SEQ ID NO: 1. In some embodiments, the modified receptor binding domain comprises an amino acid substitution corresponding to D1289Y in SEQ ID NO: 1.

In some embodiments, the modified BoNT comprising a modified protease domain further comprises a modified receptor binding domain comprising an amino acid substitution at a position corresponding to 1292 in SEQ ID NO: 1. In some embodiments, the modified receptor binding domain comprises an amino acid substitution corresponding to G1292R or G1292K in SEQ ID NO: 1.

In some embodiments, the modified BoNT comprising a modified protease domain further comprises a modified receptor binding domain comprising an amino acid substitution at a position corresponding to R1294S in SEQ ID NO: 1. In some embodiments, the modified receptor binding domain comprises an amino acid substitution corresponding to R1294S or R1294T in SEQ ID NO: 1.

In some embodiments, the modified BoNT comprising a modified protease domain further comprises a modified receptor binding domain comprising an amino acid substitution at a position corresponding to 1295 in SEQ ID NO: 1. In some embodiments, the modified receptor binding domain comprises an amino acid substitution corresponding to P1295S or P1295T in SEQ ID NO: 1.

In some embodiments, the modified BoNT comprising a modified protease domain further comprises a modified receptor binding domain comprising an amino acid substitution at a position corresponding to 1156 in SEQ ID NO: 1. In some embodiments, the modified receptor binding domain comprises an amino acid substitution corresponding to R1156M or R1156I in SEQ ID NO: 1. In some embodiments, the modified BoNT comprising a modified protease domain further comprises a modified receptor binding domain comprises SEQ ID NO: 177 or SEQ ID NO: 218. In some embodiments, the modified BoNT comprising a modified protease domain further comprises a modified receptor binding domain consists of SEQ ID NO: 177 or SEQ ID NO: 218.

Some aspects of the present disclosure provide modified Clostridial Botulinum neurotoxin (BoNT) polypeptides comprising any one of the modified protease domains described herein and a modified receptor binding domain of Clostridial Botulinum serotype A2 (BoNT/A2). In some embodiments, the modified receptor binding domain of BoNT/A2 comprises one or more amino acid substitutions at positions corresponding to 915, 923, 1090, 1103, 1117, 1156, 1170, 1227, 1254, 1255, or 1256 in SEQ ID NO: 2. in SEQ ID NO: 2. In some embodiments, the one or more amino acid substitutions in the modified receptor binding domain of BoNT/A2 correspond to one or more of K915Q, T923K, 51090N, N1103D, F1117Y, E1156M, E1170K, D1227N, L1254Q, Y1255G, or D1256N in SEQ ID NO: 2.

In some embodiments, the modified BoNT comprising a modified protease domain further comprises a modified receptor binding domain comprising an amino acid substitution at a position corresponding to 915 in SEQ ID NO: 2. In some embodiments, the modified receptor binding domain comprises an amino acid substitution corresponding to K915Q in SEQ ID NO: 2.

In some embodiments, the modified BoNT comprising a modified protease domain further comprises a modified receptor binding domain comprising an amino acid substitution at a position corresponding to 923 in SEQ ID NO: 2. In some embodiments, the modified receptor binding domain comprises an amino acid substitution corresponding to T923K in SEQ ID NO: 2.

In some embodiments, the modified BoNT comprising a modified protease domain further comprises a modified receptor binding domain comprising an amino acid substitution at a position corresponding to 1090 in SEQ ID NO: 2. In some embodiments, the modified receptor binding domain comprises an amino acid substitution corresponding to 51090N in SEQ ID NO: 2.

In some embodiments, the modified BoNT comprising a modified protease domain further comprises a modified receptor binding domain comprising an amino acid substitution at a position corresponding to 1103 in SEQ ID NO: 2. In some embodiments, the modified receptor binding domain comprises an amino acid substitution corresponding to N1103D in SEQ ID NO: 2.

In some embodiments, the modified BoNT comprising a modified protease domain further comprises a modified receptor binding domain comprising an amino acid substitution at a position corresponding to 1117 in SEQ ID NO: 2. In some embodiments, the modified receptor binding domain comprises an amino acid substitution corresponding to F1117Y in SEQ ID NO: 2.

In some embodiments, the modified BoNT comprising a modified protease domain further comprises a modified receptor binding domain comprising an amino acid substitution at a position corresponding to 1156 in SEQ ID NO: 2. In some embodiments, the modified receptor binding domain comprises an amino acid substitution corresponding to E1156M in SEQ ID NO: 2.

In some embodiments, the modified BoNT comprising a modified protease domain further comprises a modified receptor binding domain comprising an amino acid substitution at a position corresponding to 1170 in SEQ ID NO: 2. In some embodiments, the modified receptor binding domain comprises an amino acid substitution corresponding to E1170K in SEQ ID NO: 2.

In some embodiments, the modified BoNT comprising a modified protease domain further comprises a modified receptor binding domain comprising an amino acid substitution at a position corresponding to 1227 in SEQ ID NO: 2. In some embodiments, the modified receptor binding domain comprises an amino acid substitution corresponding to D1227N in SEQ ID NO: 2.

In some embodiments, the modified BoNT comprising a modified protease domain further comprises a modified receptor binding domain comprising an amino acid substitution at a position corresponding to 1254 in SEQ ID NO: 2. In some embodiments, the modified receptor binding domain comprises an amino acid substitution corresponding to L1254Q in SEQ ID NO: 2.

In some embodiments, the modified BoNT comprising a modified protease domain further comprises a modified receptor binding domain comprising an amino acid substitution at a position corresponding to 1255 in SEQ ID NO: 2. In some embodiments, the modified receptor binding domain comprises an amino acid substitution corresponding to Y1255F in SEQ ID NO: 2.

In some embodiments, the modified BoNT comprising a modified protease domain further comprises a modified receptor binding domain comprising an amino acid substitution at a position corresponding to 1256 in SEQ ID NO: 2. In some embodiments, the modified receptor binding domain comprises an amino acid substitution corresponding to D1256N in SEQ ID NO: 2.

In some embodiments, the modified BoNT polypeptides of the present comprise a protease domain (LC), a linker region, a translocation domain (H_{N}), and a receptor binding domain (H_{C}), wherein the linker region is located between the protease domain and the translocation domain. BoNTs are initially produced as single polypeptide and activated via the cleavage of the linker region between LC and HN either bacterial or host proteases. This process is known as “activation” and is essential for the activity the modified BoNT polypeptides described herein. After the cleavage, the LC and H remain connected via an inter-chain disulfide bond prior to translocation of LC into the cytosol of cells, where the disulfide bond is reduced in order to release the LC into the cytosol.

Further contemplated herein are modified BoNT polypeptides comprising a modified linker region, wherein the linker region comprises a specific protease cleavage site. A “specific protease cleavage site,” as used herein, refers to a recognition and cleavage site for a specification protease, as opposed to a sequence that is recognized and cleavage by more than one non-specific proteases. Such specific proteases include, without limitation: thrombin, TEV, PreScission, Factor Xa, MMP-12, MMP-13, MMP-17, MMP-20, Granzyme-B, and Enterokinase.

In some embodiments, the cleavage site of the specific proteases may be added to the linker region of a modified BoNT polypeptide described herein (e.g., SEQ ID NOs: 2-34, 36-43, 45-77, 79-86, 88-120, 122-129, 131-203, 213-216) via insertion or replacement of the existing amino acids in the linker region (e.g. replace amino acids 430-454 of modified BoNT polypeptides comprising the LC-H_{N} domain of BoNT/A1 and amino acids 430-454 of modified BoNT polypeptides comprising the LC-H_{N} domain of BoNT/A2). The sequences of the specific protease cleavage sites sequences are also provided: LVPR|GS (thrombin, SEQ ID NO: 206), ENLYFQG (TEV, SEQ ID NO: 207), LEVLFQ|GP (PreScission, SEQ ID NO: 208), IEGR| or IDGR|(Factor Xa, SEQ ID NO: 209 or 210), DDDDK| (Enterokinase, SEQ ID NO: 211) and AHREQIGG| (SUMO protease, SEQ ID NO: 212). “|” indicates where cleavage occurs.

The modified BoNT polypeptides of the present disclosure (e.g., without limitation, polypeptides comprising amino acid sequence of any of SEQ ID NOs: 2-34, 36-43, 45-77, 79-86, 88-120, 122-129, 131-203, 213-216), will generally be produced by expression form recombinant nucleic acids in appropriate cells (e.g., E. coli, or insect cells) and isolated. The nucleic acids encoding the polypeptides described herein may be obtained, and the nucleotide sequence of the nucleic acids determined, by any method known in the art.

Further provided herein are isolated and/or recombinant nucleic acids encoding any of the modified BoNT polypeptides disclosed herein. The nucleic acids encoding the isolated polypeptide fragments of the present disclosure, may be DNA or RNA, double-stranded or single stranded. In certain aspects, the subject nucleic acids encoding the isolated polypeptide fragments are further understood to include nucleic acids encoding polypeptides that are variants of any of the modified BoNT polypeptides described herein.

Variant nucleotide sequences include sequences that differ by one or more nucleotide substitutions, additions or deletions, such as allelic variants. In some embodiments, the isolated nucleic acid molecule of the present disclosure comprising a polynucleotide encoding a polypeptide comprising an amino acid sequence that has at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identity of any of SEQ ID NOs: 2-34, 36-43, 45-77, 79-86, 88-120, 122-129, 131-203, 213-215. In some embodiments, the isolated nucleic acid molecule of the present disclosure comprising a polynucleotide encoding a polypeptide comprising an amino acid sequence that has 85%, 86%, 87%, 88%, 89%, 90%, 91%, 92%, 93%, 94%, 95%, 96%, 97%, 98%, 99%, or 100% identity of any of SEQ ID NOs: 2-34, 36-43, 45-77, 79-86, 88-120, 122-129, 131-203, 213-215.

In some embodiments, the nucleic acid is comprised within a vector, such as an expression vector. In some embodiments, the vector comprises a promoter operably linked to the nucleic acid.

A variety of promoters can be used for expression of the polypeptides described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)].

Other systems include FK506 dimer, VP16 or p65 using astradiol, RU486, diphenol murislerone, or rapamycin. Inducible systems are available from Invitrogen, Clontech and Ariad. Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter.

The host cells used to express the isolated polypeptides described herein may be either bacterial cells such as Escherichia coli, or, preferably, eukaryotic cells. In particular, mammalian cells, such as Chinese hamster ovary cells (CHO), in conjunction with a vector such as the major intermediate early gene promoter element from human cytomegalovirus is an effective expression system for immunoglobulins (Foecking et al. (1986) “Powerful And Versatile Enhancer-Promoter Unit For Mammalian Expression Vectors,” Gene 45:101-106; Cockett et al. (1990) “High Level Expression Of Tissue Inhibitor Of Metalloproteinases In Chinese Hamster Ovary Cells Using Glutamine Synthetase Gene Amplification,” Biotechnology 8:662-667). A variety of host-expression vector systems may be utilized to express the isolated polypeptides described herein. Such host-expression systems represent vehicles by which the coding sequences of the isolate d polypeptides described herein may be produced and subsequently purified, but also represent cells which may, when transformed or transfected with the appropriate nucleotide coding sequences, express the isolated polypeptides described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the isolated polypeptides described herein; yeast (e.g., Saccharomyces pichia) transformed with recombinant yeast expression vectors containing sequences encoding the isolated polypeptides described herein; insect cell systems infected with recombinant virus expression vectors (e.g., baclovirus) containing the sequences encoding the isolated polypeptides described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the isolated polypeptides described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the polypeptides being expressed. For example, when a large quantity of such a protein is to be produced, for the generation of pharmaceutical compositions of polypeptides described herein, vectors which direct the expression of high levels of fusion protein products that are readily purified may be desirable. Such vectors include, but are not limited, to the E. coli expression vector pUR278 (Rather et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The 1pp Gene Of Escherichia Coli,” Nucleic Acids Res. 13:3101-3110; Van Heeke et al. (1989) “Expression Of Human Asparagine Synthetase In Escherichia Coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione.

The pGEX vectors are designed to include thrombin or factor Xa protease cleavage sites so that the cloned target gene product can be released from the GST moiety. In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic.

The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544). In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. For example, in certain embodiments, the polypeptides described herein may be expressed as a single gene product (e.g., as a single polypeptide chain, i.e., as a polyprotein precursor), requiring proteolytic cleavage by native or recombinant cellular mechanisms to form separate polypeptides described herein.

The disclosure thus encompasses engineering a nucleic acid sequence to encode a polyprotein precursor molecule comprising the polypeptides described herein, which includes coding sequences capable of directing post translational cleavage of said polyprotein precursor. Post-translational cleavage of the polyprotein precursor results in the polypeptides described herein. The post translational cleavage of the precursor molecule comprising the polypeptides described herein may occur in vivo (i.e., within the host cell by native or recombinant cell systems/mechanisms, e.g. furin cleavage at an appropriate site) or may occur in vitro (e.g. incubation of said polypeptide chain in a composition comprising proteases or peptidases of known activity and/or in a composition comprising conditions or reagents known to foster the desired proteolytic action).

Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa (Nagai et al. (1985) “Oxygen Binding Properties Of Human Mutant Hemoglobins Synthesized In Escherichia Coli,” Proc. Nat. Acad. Sci. USA 82:7252-7255, and reviewed in Jenny et al. (2003) “A Critical Review Of The Methods For Cleavage Of Fusion Proteins With Thrombin And Factor Xa,” Protein Expr. Purif. 31:1-11, each of which is incorporated by reference herein in its entirety)), enterokinase (Collins-Racie et al. (1995) “Production Of Recombinant Bovine Enterokinase Catalytic Subunit In Escherichia Coli Using The Novel Secretory Fusion Partner DsbA,” Biotechnology 13:982-987 hereby incorporated by reference herein in its entirety)), furin, and AcTEV (Parks et al. (1994) “Release Of Proteins And Peptides From Fusion Proteins Using A Recombinant Plant Virus Proteinase,” Anal. Biochem. 216:413-417 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express polypeptides described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptides described herein. Such engineered cell lines may be particularly useful in screening and evaluation of polypeptides that interact directly or indirectly with the polypeptides described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11: 223-232), hypoxanthine-guanine phosphoribosyltransferase (Szybalska et al. (1992) “Use Of The HPRT Gene And The HAT Selection Technique In DNA-Mediated Transformation Of Mammalian Cells First Steps Toward Developing Hybridoma Techniques And Gene Therapy,” Bioessays 14: 495-500), and adenine phosphoribosyltransferase (Lowy et al. (1980) “Isolation Of Transforming DNA: Cloning The Hamster aprt Gene,” Cell 22: 817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78: 1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78: 2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al. (1981) “A New Dominant Hybrid Selective Marker For Higher Eukaryotic Cells,” J. Mol. Biol. 150:1-14.

The expression levels of polypeptides described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a polypeptide described herein is amplifiable, increase in the level of inhibitor present in culture of host cell will increase the number of copies of the marker gene. Since the amplified region is associated with the nucleotide sequence of a polypeptide described herein or a polypeptide described herein, production of the polypeptide will also increase (Crouse et al. (1983) “Expression And Amplification Of Engineered Mouse Dihydrofolate Reductase Minigenes,” Mol. Cell. Biol. 3:257-266).

Once a polypeptide described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (in some embodiments after Protein A selection where the polypeptide comprises an Fc domain (or portion thereof)), and sizing column chromatography), centrifugation, differential solubility, or by any other standard technique for the purification of polypeptides or antibodies. Other aspects of the present disclosure relate to a cell comprising a nucleic acid described herein or a vector described herein.

The cell may be a prokaryotic or eukaryotic cell. In some embodiments, the cell in a mammalian cell. Exemplary cell types are described herein. Other aspects of the present disclosure related to a cell expressing the modified BoNT polypeptides described herein. The cell may be a prokaryotic or eukaryotic cell. In some embodiments, the cell in a mammalian cell. Exemplary cell types are described herein. The cell can be for propagation of the nucleic acid or for expression of the nucleic acid, or both. Such cells include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection, European Collection of Cell Cultures and the German Collection of Microorganisms and Cell Cultures. Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4.sup.th ed. 2000); ANIMAL CELL CULTURE: A PRACTICAL APPROACH (John R. W. Masters ed., Oxford University Press, 3.sup.rd ed. 2000); MOLECULAR CLONING A LABORATORY MANUAL, supra, (2001); BASIC CELL CULTURE: A PRACTICAL APPROACH (John M. Davis, Oxford Press, 2.sup.nd ed. 2002); and CURRENT PROTOCOLS IN MOLECULAR BIOLOGY, supra, (2004).

These protocols are routine procedures within the scope of one skilled in the art and from the teaching herein. Yet other aspects of the present disclosure relate to a method of producing a polypeptide described herein, the method comprising obtaining a cell described herein and expressing nucleic acid described herein in said cell. In some embodiments, the method further comprises isolating and purifying a polypeptide described herein.

In some embodiments, botulinum neurotoxin can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive.

The bacterial strains that make botulinum toxin serotypes A and G possess endogenous proteases and serotypes A and G can therefore be recovered from bacterial cultures in predominantly their active form. In contrast, botulinum toxin serotypes Ci, D and E are synthesized by non-proteolytic strains and are therefore typically unactivated when recovered from culture. Serotypes B and F are produced by both proteolytic and non-proteolytic strains and therefore can be recovered in either the active or inactive form. The proteolytic strains that produce, for example, the botulinum toxin type A serotype may only cleave a portion of the toxin produced.

The exact proportion of nicked to un-nicked molecules depends on the length of incubation and the temperature of the culture. Therefore, a certain percentage of a preparation of, for example, the botulinum toxin type A toxin may be inactive. In one embodiment, the neurotoxin of the present disclosure is in an active state. In one embodiment, the neurotoxin is in an inactive state. In one embodiment, a combination of active and inactive neurotoxin is envisioned.

Thus, the present disclosure also contemplates pharmaceutically compositions comprising the modified BoNTs or the chimeric molecules of the present disclosure. As it may also become clear later in the present disclosure, the pharmaceutical composition of the present disclosure, may further comprise other therapeutic agents suitable for the specific disease such composition is designed to treat. In some embodiments, the pharmaceutically composition of the present disclosure further comprises pharmaceutically-acceptable carriers.

The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the polypeptide from one site (e.g., the delivery site) of the body, to another site (e.g., organ, tissue or portion of the body).

A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethylcellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, a modified BoNT polypeptide of the present disclosure in a composition is administered by injection, by means of a catheter, by means of a suppository, or by means of an implant, the implant being of a porous, non-porous, or gelatinous material, including a membrane, such as a sialastic membrane, or a fiber.

Typically, when administering the composition, materials to which the polypeptide of the disclosure does not absorb are used. In other embodiments, the modified BoNT polypeptides of the present disclosure are delivered in a controlled release system. Such compositions and methods for administration are provides in U.S. Patent publication No. 2007/0020295, the contents of which are herein incorporated by reference. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

The modified BoNT polypeptides of the present disclosure can be administered as pharmaceutical compositions comprising a therapeutically effective amount of a binding agent and one or more pharmaceutically compatible ingredients. In typical embodiments, the pharmaceutical composition is formulated in accordance with routine procedures as a pharmaceutical composition adapted for intravenous or subcutaneous administration to a subject, e.g., a human being.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration. A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein.

The polypeptides of the present disclosure can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757. The pharmaceutical compositions of the present disclosure may be administered or packaged as a unit dose, for example.

The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle. In some embodiments, the modified BoNT polypeptides described herein may be conjugated to a therapeutic moiety, e.g., an antibiotic. Techniques for conjugating such therapeutic moieties to polypeptides, including e.g., Fc domains, are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev., 62:119-158. Further, the pharmaceutical composition can be provided as a pharmaceutical kit comprising (a) a container containing a polypeptide of the disclosure in lyophilized form and (b) a second container containing a pharmaceutically acceptable diluent (e.g., sterile water) for injection. The pharmaceutically acceptable diluent can be used for reconstitution or dilution of the lyophilized polypeptide of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration. In another aspect, an article of manufacture containing materials useful for the treatment of the diseases described above is included. In some embodiments, the article of manufacture comprises a container and a label.

Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an isolated polypeptide of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

The modified BoNT polypeptides, the chimeric molecules, and the pharmaceutical compositions of the present disclosure may be used for the treatment of conditions associated with unwanted neuronal activities. Thus, further provided herein are methods of treating a condition associated with unwanted neuronal activity, the method comprising administering a therapeutically effective amount of the modified BoNT polypeptide, the chimeric molecule, or the pharmaceutical composition described herein to thereby treat the condition. In some embodiments, the modified BoNT polypeptides, the chimeric molecules, and the pharmaceutic compositions of the present disclosure contact one or more neuron(s) exhibiting unwanted neuronal activity,

Condition typically treated with a neurotoxin (e.g., skeletal muscle conditions, smooth muscle conditions, glandular conditions, a neuromuscular disorder, an autonomic disorder, pain, or an aesthetic/cosmetic condition) are associated with unwanted neuronal activity, as determined by the skilled practitioner. Administration is by a route that contacts an effective amount of the composition to neurons exhibiting the unwanted activity. In some embodiments, the condition may be associated with overactive neurons or glands. Specific conditions envisioned for treatment by the methods discussed herein include, without limitation, spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions as well as other secretory disorders, pain from muscle spasms, headache pain. In addition, the present disclosure can be used to treat dermato logical or aesthetic/cosmetic conditions, for example, reduction of brow furrows, reduction of skin wrinkles.

The present disclosure can also be used in the treatment of sports injuries. Borodic U.S. Pat. No. 5,053,005 discloses methods for treating juvenile spinal curvature, i.e. scoliosis, using botulinum type A. The disclosure of Borodic is incorporated in its entirety herein by reference. In one embodiment, using substantially similar methods as disclosed by Borodic, a modified neurotoxin can be administered to a mammal, preferably a human, to treat spinal curvature. In a suitable embodiment, a modified neurotoxin comprising botulinum type E fused with a leucine-based motif is administered. Even more preferably, a modified neurotoxin comprising botulinum type A-E with a leucine-based motif fused to the carboxyl terminal of its light chain is administered to the mammal, preferably a human, to treat spinal curvature.

In addition, the modified neurotoxin can be administered to treat other neuromuscular disorders using well known techniques that are commonly performed with botulinum type A. For example, the present disclosure can be used to treat pain, for example, headache pain, pain from muscle spasms and various forms of inflammatory pain. For example, Aoki U.S. Pat. No. 5,721,215 and Aoki U.S. Pat. No. 6,113,915 disclose methods of using botulinum toxin type A for treating pain. The disclosure of these two patents is incorporated in its entirety herein by reference.

Autonomic nervous system disorders can also be treated with a modified neurotoxin. For example, glandular malfunctioning is an autonomic nervous system disorder. Glandular malfunctioning includes excessive sweating and excessive salivation. Respiratory malfunctioning is another example of an autonomic nervous system disorder. Respiratory malfunctioning includes chronic obstructive pulmonary disease and asthma. Sanders et al. disclose methods for treating the autonomic nervous system; for example, treating autonomic nervous system disorders such as excessive sweating, excessive salivation, asthma, etc., using naturally existing botulinum toxins. The disclosure of Sander et al. is incorporated in its entirety by reference herein.

In one embodiment, substantially similar methods to that of Sanders et al. can be employed, but using a modified neurotoxin, to treat autonomic nervous system disorders such as the ones discussed above. For example, a modified neurotoxin can be locally applied to the nasal cavity of the mammal in an amount sufficient to degenerate cholinergic neurons of the autonomic nervous system that control the mucous secretion in the nasal cavity. Pain that can be treated by a modified neurotoxin includes pain caused by muscle tension, or spasm, or pain that is not associated with muscle spasm. For example, Binder in U.S. Pat. No. 5,714,468 discloses that headache caused by vascular disturbances, muscular tension, neuralgia and neuropathy can be treated with a naturally occurring botulinum toxin, for example Botulinum type A. The disclosures of Binder are incorporated in its entirety herein by reference.

In one embodiment, substantially similar methods to that of Binder can be employed, but using a modified neurotoxin, to treat headache, especially the ones caused by vascular disturbances, muscular tension, neuralgia and neuropathy. Pain caused by muscle spasm can also be treated by an administration of a modified neurotoxin. For example, a botulinum type E fused with a leucine-based motif, preferably at the carboxyl terminal of the botulinum type E light chain, can be administered intramuscularly at the pain/spasm location to alleviate pain. Furthermore, a modified neurotoxin can be administered to a mammal to treat pain that is not associated with a muscular disorder, such as spasm.

In one broad embodiment, methods of the present disclosure to treat non-spasm related pain include central administration or peripheral administration of the modified neurotoxin. For example, Foster et al. in U.S. Pat. No. 5,989,545 discloses that a botulinum toxin conjugated with a targeting moiety can be administered centrally (intrathecally) to alleviate pain. The disclosures of Foster et al. are incorporated in its entirety by reference herein.

In one embodiment, substantially similar methods to that of Foster et al. can be employed, but using the compositions described herein to treat pain. The pain to be treated can be an acute pain or chronic pain. An acute or chronic pain that is not associated with a muscle spasm can also be alleviated with a local, peripheral administration of the modified neurotoxin to an actual or a perceived pain location on the mammal.

In one embodiment, the modified neurotoxin is administered subcutaneously at or near the location of pain, for example, at or near a cut. In some embodiments, the modified neurotoxin is administered intramuscularly at or near the location of pain, for example, at or near a bruise location on the mammal. In some embodiments, the modified BoNT polypeptide is injected directly into a joint of a mammal, for treating or alleviating pain caused by arthritic conditions. Also, frequent repeated injection or infusion of the modified neurotoxin to a peripheral pain location is within the scope of the present disclosure. Routes of administration for such methods are known in the art and easily adapted to the methods described herein by the skilled practitioner (e.g., see for example, Harrison's Principles of Internal Medicine (1998), edited by Anthony Fauci et al., 14.sup.th edition, published by McGraw Hill).

By way of non-limiting example, the treatment of a neuromuscular disorder can comprise a step of locally administering an effective amount of the molecule to a muscle or a group of muscles, the treatment of an autonomic disorder can comprise a step of locally administering an effective of the molecule to a gland or glands, and the treatment of pain can comprise a step of administering an effective amount of the molecule the site of the pain. In addition, the treatment of pain can comprise a step of administering an effective amount of a modified neurotoxin to the spinal cord.

“A therapeutically effective amount” as used herein refers to the amount of each therapeutic agent of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons. Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system.

Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including the polypeptide used) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide (such as the half-life of the polypeptide, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the polypeptide is administered for preventive or therapeutic purposes, previous therapy, the subject's clinical history and response to the antagonist, and the discretion of the attending physician. Typically the clinician will administer a polypeptide until a dosage is reached that achieves the desired result.

Administration of one or more polypeptides can be continuous or intermittent, depending, for example, upon the recipient's physiological condition, whether the purpose of the administration is therapeutic or prophylactic, and other factors known to skilled practitioners. The administration of a polypeptide may be essentially continuous over a preselected period of time or may be in a series of spaced dose, e.g., either before, during, or after developing a disease. As used herein, the term “treating” refers to the application or administration of a polypeptide or composition including the polypeptide to a subject in need thereof.

“A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has CDI. In some embodiments, the subject has cancer. In some embodiments, the subject is a mammal. In some embodiments, the subject is a non-human primate. In some embodiments, the subject is human. Alleviating a disease includes delaying the development or progression of the disease, or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

As used herein, a “subject” refers to a human or animal. Usually, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human.

The terms, “patient” and “subject” are used interchangeably herein. A subject can be male or female. A subject can be a fully developed subject (e.g., an adult) or a subject undergoing the developmental process (e.g., a child, infant or fetus). Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with unwanted neuronal activity. In addition, the methods and compositions described herein can be used to treat domesticated animals and/or pets.

### REFERENCES: DETAILED DESCRIPTION

1. Schiavo, G., Matteoli, M. & Montecucco, C. Neurotoxins affecting neuroexocytosis. Physiol Rev 80, 717-766 (2000).
2. Dong, M., Masuyer, G. & Stenmark, P. Botulinum and Tetanus Neurotoxins. Annu Rev Biochem 88, 811-837 (2019).
3. Johnson, E. A. Clostridial toxins as therapeutic agents: benefits of nature's most toxic proteins. Annu Rev Microbiol 53, 551-575 (1999).
4. Aoki, K. R. Botulinum toxin: a successful therapeutic protein. Curr Med Chem 11, 3085-3092 (2004).
5. Montecucco, C. & Molgo, J. Botulinal neurotoxins: revival of an old killer. Curr Opin Pharmacol 5, 274-279 (2005).
6. Lange, O., Bigalke, H., Dengler, R., Wegner, F., deGroot, M. & Wohlfarth, K. Neutralizing antibodies and secondary therapy failure after treatment with botulinum toxin type A: much ado about nothing? Clin Neuropharmacol 32, 213-218 (2009).
7. Chapman, M. A., Barron, R., Tanis, D. C., Gill, C. E. & Charles, P. D. Comparison of botulinum neurotoxin preparations for the treatment of cervical dystonia. Clin Ther 29, 1325-1337 (2007).
8. Cote, T. R., Mohan, A. K., Polder, J. A., Walton, M. K. & Braun, M. M. Botulinum toxin type A injections: adverse events reported to the US Food and Drug Administration in therapeutic and cosmetic cases. J Am Acad Dermatol 53, 407-415 (2005).
9. Dong, M., Tepp, W. H., Liu, H., Johnson, E. A. & Chapman, E. R. Mechanism of botulinum neurotoxin B and G entry into hippocampal neurons. J Cell Biol 179, 1511-1522 (2007).
10. Peng, L., Tepp, W. H., Johnson, E. A. & Dong, M. Botulinum neurotoxin D uses synaptic vesicle protein SV2 and gangliosides as receptors. PLoS Pathog 7, e1002008 (2011).
11. Dong, M., Richards, D. A., Goodnough, M. C., Tepp, W. H., Johnson, E. A. & Chapman, E. R. Synaptotagmins I and II mediate entry of botulinum neurotoxin B into cells. J Cell Biol 162, 1293-1303 (2003).
12. Nishiki, T., Kamata, Y., Nemoto, Y., Omori, A., Ito, T., Takahashi, M. & Kozaki, S. Identification of protein receptor for Clostridium botulinum type B neurotoxin in rat brain synaptosomes. J Biol Chem 269, 10498-10503 (1994).
13. Rummel, A., Karnath, T., Henke, T., Bigalke, H. & Binz, T. Synaptotagmins I and II act as nerve cell receptors for botulinum neurotoxin G. J Biol Chem 279, 30865-30870 (2004).
14. Peng, L., Berntsson, R. P., Tepp, W. H., Pitkin, R. M., Johnson, E. A., Stenmark, P. & Dong, M. Botulinum neurotoxin D-C uses synaptotagmin I/II as receptors and human synaptotagmin II is not an effective receptor for type B, D-C, and G toxins. J Cell Sci (2012).
15. Jin, R., Rummel, A., Binz, T. & Brunger, A. T. Botulinum neurotoxin B recognizes its protein receptor with high affinity and specificity. Nature 444, 1092-1095 (2006).
16. Chai, Q., Arndt, J. W., Dong, M., Tepp, W. H., Johnson, E. A., Chapman, E. R. & Stevens, R. C. Structural basis of cell surface receptor recognition by botulinum neurotoxin B. Nature 444, 1096-1100 (2006).
17. Benoit, R. M., Frey, D., Hilbert, M., Kevenaar, J. T., Wieser, M. M., Stirnimann, C. U., McMillan, D., Ceska, T., Lebon, F., Jaussi, R., Steinmetz, M. O., Schertler, G. F., Hoogenraad, C. C., Capitani, G. & Kammerer, R. A. Structural basis for recognition of synaptic vesicle protein 2C by botulinum neurotoxin A. Nature 505, 108-111 (2014).
18. Dong, M., Liu, H., Tepp, W. H., Johnson, E. A., Janz, R. & Chapman, E. R. Glycosylated SV2A and SV2B mediate the entry of botulinum neurotoxin E into neurons. Mol Biol Cell 19, 5226-5237 (2008).
19. Dong, M., Yeh, F., Tepp, W. H., Dean, C., Johnson, E. A., Janz, R. & Chapman, E. R. SV2 is the protein receptor for botulinum neurotoxin A. Science 312, 592-596 (2006).
20. Mahrhold, S., Rummel, A., Bigalke, H., Davletov, B. & Binz, T. The synaptic vesicle protein 2C mediates the uptake of botulinum neurotoxin A into phrenic nerves. FEBS Lett 580, 2011-2014 (2006).
21. Rummel, A., Hafner, K., Mahrhold, S., Darashchonak, N., Holt, M., Jahn, R., Beermann, S., Karnath, T., Bigalke, H. & Binz, T. Botulinum neurotoxins C, E and F bind gangliosides via a conserved binding site prior to stimulation-dependent uptake with botulinum neurotoxin F utilising the three isoforms of SV2 as second receptor. J Neurochem 110, 1942-1954 (2009).
22. Fu, Z., Chen, C., Barbieri, J. T., Kim, J. J. & Baldwin, M. R. Glycosylated SV2 and gangliosides as dual receptors for botulinum neurotoxin serotype F. Biochemistry 48, 5631-5641 (2009).
23. Montecucco, C. How do tetanus and botulinum toxins bind to neuronal membranes? TIBS 11, 314-317 (1986).
24. Peck, M. W., Smith, T. J., Anniballi, F., Austin, J. W., Bano, L., Bradshaw, M., Cuervo, P., Cheng, L. W., Derman, Y., Dorner, B. G., Fisher, A., Hill, K. K., Kalb, S. R., Korkeala, H., Lindstrom, M., Lista, F., Luquez, C., Mazuet, C., Pirazzini, M., Popoff, M. R., Rossetto, O., Rummel, A., Sesardic, D., Singh, B. R. & Stringer, S. C. Historical Perspectives and Guidelines for Botulinum Neurotoxin Subtype Nomenclature. Toxins (Basel) 9(2017).
25. Pier, C. L., Chen, C., Tepp, W. H., Lin, G., Janda, K. D., Barbieri, J. T., Pellett, S. & Johnson, E. A. Botulinum neurotoxin subtype A2 enters neuronal cells faster than subtype A1. FEBS Lett 585, 199-206 (2011).
26. Yao, G., Zhang, S., Mahrhold, S., Lam, K. H., Stern, D., Bagramyan, K., Perry, K., Kalkum, M., Rummel, A., Dong, M. & Jin, R. N-linked glycosylation of SV2 is required for binding and uptake of botulinum neurotoxin A. Nat Struct Mol Biol 23, 656-662 (2016).
27. Barash, J. R. & Arnon, S. S. A novel strain of Clostridium botulinum that produces type B and type H botulinum toxins. J Infect Dis 209, 183-191 (2014).
28. Zhang, S., Masuyer, G., Zhang, J., Shen, Y., Lundin, D., Henriksson, L., Miyashita, S. I., Martinez-Carranza, M., Dong, M. & Stenmark, P. Identification and characterization of a novel botulinum neurotoxin. Nat Commun 8, 14130 (2017).
29. Popp, M. W., Antos, J. M., Grotenbreg, G. M., Spooner, E. & Ploegh, H. L. Sortagging: a versatile method for protein labeling. Nat Chem Biol 3, 707-708 (2007).
30. Aoki, K. R. A comparison of the safety margins of botulinum neurotoxin serotypes A, B, and F in mice. Toxicon 39, 1815-1820 (2001).
31. Arnon, S. S., Schechter, R., Inglesby, T. V., Henderson, D. A., Bartlett, J. G., Ascher, M. S., Eitzen, E., Fine, A. D., Hauer, J., Layton, M., Lillibridge, S., Osterholm, M. T., O'Toole, T., Parker, G., Perl, T. M., Russell, P. K., Swerdlow, D. L. & Tonat, K. Botulinum toxin as a biological weapon: medical and public health management. Jama 285, 1059-1070 (2001).
32. Montal, M. Botulinum neurotoxin: a marvel of protein design. Annu Rev Biochem 79, 591-617 (2010).
33. Rossetto, O., Pirazzini, M. & Montecucco, C. botulinum neurotoxins: genetic, structural and mechanistic insights. Nat Rev Microbiol 12, 535-549 (2014).
34. Pirazzini, M., et al. Thioredoxin and its reductase are present on synaptic vesicles, and their inhibition prevents the paralysis induced by botulinum neurotoxins. Cell Rep 8, 1870-1878 (2014).
35. Pirazzini, M., et al. The thioredoxin reductase—Thioredoxin redox system cleaves the interchain disulphide bond of botulinum neurotoxins on the cytosolic surface of synaptic vesicles. Toxicon 107, 32-36 (2015).
36. JaHN, R. & Scheller, R. H. SNAREs—engines for membrane fusion. Nat Rev Mol Cell Biol 7, 631-643 (2006).
37. Sudhof, T. C. & Rothman, J. E. Membrane fusion: grappling with SNARE and SM proteins. Science 323, 474-477 (2009).
38. Gu, S., et al. Botulinum neurotoxin is shielded by NTNHA in an interlocked complex. Science 335, 977-981 (2012).
39. Lee, K., et al. Molecular basis for disruption of E-cadherin adhesion by botulinum neurotoxin A complex. Science 344, 1405-1410 (2014).
40. Lee, K., et al. Molecular basis for disruption of E-cadherin adhesion by botulinum neurotoxin A complex. Science 344, 1405-1410 (2014).
41. Sugawara, Y., et al. Botulinum hemagglutinin disrupts the intercellular epithelial barrier by directly binding E-cadherin. J Cell Biol 189, 691-700 (2010).
42. Hill, K. K., Xie, G., Foley, B. T. & Smith, T. J. Genetic diversity within the botulinum neurotoxin-producing bacteria and their neurotoxins. Toxicon 107, 2-8 (2015).

### EXAMPLES

### Example 1

Botulinum neurotoxins (BoNTs) are a family of bacterial toxins that target and enter nerve terminals and then act as a protease to cleave key proteins required for neurotransmitter release, thus inducing flaccid paralysis of muscles. Members of BoNTs, such as BoNT/A, have been utilized to treat a wide range of medical conditions as well as for cosmetic purposes of reducing wrinkles. A local injection of a tiny amount of BoNT/A can induce relaxation of muscles for 3-6 months in humans. However, an issue with clinical use of BoNT/A is that patients may generate neutralizing antibodies. Because the chance of generating neutralizing antibodies is directly related to injected doses, lowering the toxin therapeutic doses is highly desired, which means the efficacy of individual toxin molecules has to be enhanced. Here, an approach was developed to enhance BoNT efficacy and therapeutic duration by “de-lysine” the lysine residues on the surface of the BoNT protease domain. Using this approach, BoNT/A protease domain mutants containing substitutions of surface lysine residues into arginines were systemically designed and tested. A set of mutations that significantly enhanced the efficacy than the wild-type protease domain were identified. Their enhanced efficacy was validated in the context of full-length BoNT/A on both cultured neurons and in vivo in mouse models. These improved protease domains can be utilized to create a new generation of BoNT/A and other chimeric BoNT molecules for therapeutic uses by providing enhanced therapeutic efficacy, longer therapeutic duration, and lower chances of inducing neutralizing antibodies than currently available BoNTs.

Botulinum neurotoxins are a family of bacterial toxins, including seven major serotypes (BoNT/A-G)^{1,2}. These toxins act by blocking neurotransmitter release from neurons, thus paralyzing animals and humans. In recent years, BoNTs have been widely used to treat a growing list of medical conditions: local injections of minute amount of toxins can attenuate neuronal activity in targeted regions, which can be beneficial in many medical conditions as well as for cosmetic purposes³⁻⁵.

BoNT/A and BoNT/B are the only two BoNTs that are currently FDA-approved for use in humans³⁻⁵. These are toxins purified from bacteria without any sequence modifications (defined as wild type, WT). As the application of BoNTs grows, limitations and adverse effects have been reported. The major limitation is the generation of neutralizing antibodies in patients, which renders future treatment ineffective⁶. Termination of BoNT usage often leaves patients with no other effective ways to treat/relieve their disorders. The possibility of antibody responses is directly related to both toxin doses and the frequency of injection⁶.

The major adverse effects largely due to diffusion of toxins to other regions of the body and the possibility of toxin diffusion is directly related to injected doses. The adverse effects ranges from transient non-serious events such as ptosis and diplopia to life-threatening events even death^{7,8}. In a petition letter filed in 2008 by Dr. Sidney Wolfe to FDA, a total of 180 serious adverse events, including 16 deaths have been documented. As a result, FDA now requires the “Black box warning” on all BoNT products, highlighting the risk of the spread of toxins, following similar warnings issued by the European Union.

Because both the generation of neutralizing antibodies and toxin diffusion are directly related to injected doses, lowering toxin doses (while maintaining the same levels of toxin activity) is highly desired, which means the efficacy of individual toxin molecule to induce local muscle paralysis has to be enhanced. Such modified BoNTs with improved local efficacy will also reduce any potential off-target effects due to toxin diffusion to other regions. Besides injection dose, more frequent injections also lead to higher chances of inducing neutralizing antibodies. Thus, extending therapeutic duration of each injection is another major goal of improving BoNTs for therapeutic uses. BoNTs such as BoNT/A has showed long half-life in neurons and induces persistent muscle paralysis in humans for 3-6 months. This relatively long duration is a key pharmacological property that enables medical use of BoNTs. Further extending this therapeutic duration is highly desirable, as it will reduce the frequency of repeated toxin injection, reducing both the cost for the patients and the chance of generating neutralizing antibodies.

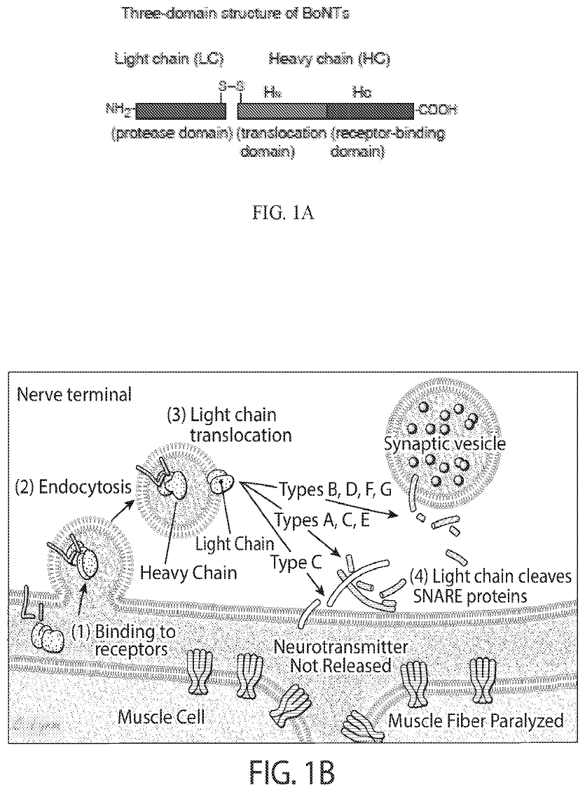

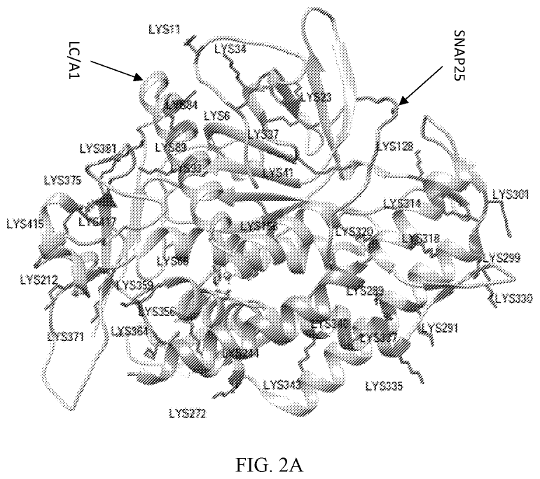

BoNTs are composed of three functional domains (FIG. 1A)²: (1) the C-terminal receptor-binding domain (H_{C}, ˜50 kDa); (2) the membrane translocation domain in the middle (H_{N}, ˜50 kDa); (3) the N-terminal protease domain (also known as light chain, LC, ˜50 kDa). Correspondingly, BoNTs has three major steps for their action (FIG. 1B): (1) receptor binding: these toxins target motor nerve terminals by first binding specifically to their receptors expressed in neurons; (2) translocation: after binding to receptors, BoNTs enter cells via receptor-mediated endocytosis into endosomes, and the low pH conditions within endosomes then induces conformational changes of toxin, resulting in its penetration of endosomal membrane and release of its protease domain into the cytosol of neurons; (3) substrate cleavage: within the cytosol of neurons, the released LC of BoNTs then cleave proteins required for synaptic transmission, therefore blocking neurotransmission².

Enhancing the efficacy of BoNTs can be achieved by improving (1) receptor-binding capability; (2) translocation efficiency; (3) LC enzymatic activity and duration. The therapeutic duration is largely determined by how fast the amount of toxin molecules falls below the effective dose inside neurons, which is a combination of two factors (1) total numbers of toxin LC entering neurons; (2) half-life of each LC inside neurons.

Previous studies have showed that the efficacy of BoNTs can be improved by enhancing receptor-bindine^{9,10} and improving translocation 11 through designed point mutations. Mutations in toxin LC that enhance its enzymatic activity 12 or oxidative stability 13 have been previously designed and evaluated, but they did not lead to any enhancement of toxin efficacy or duration compared with WT toxin.

Among the three functional domains of BoNT, LC plays critical role in determining duration of action and overall activity. Replacing the surface lysine (K) residues of BoNT-LCs to similar arginine (R) residues may improve LCs for the following rationales: (1) lysine is the site for ubiquitination, which leads to degradation of LCs in cells through ubiquitin-proteasome pathway. Reducing the numbers of lysine in LCs (“de-lysine”) can decrease its chance to be ubiquitinated, thus potentially extending its half-life. (2) Arginine, which is a positively charged amino acid similar to lysine, has higher pKa value than lysine. Arginine maintains its charge during membrane translocation, whereas lysine is readily deprotonated to become a neutral residue in the membrane. Therefore, proteins containing arginine would be easier to translocate through membranes than proteins containing lysines.

The approach to “de-lysine” mutant LCs was a combination of structure-assisted design and consensus mutagenesis based on naturally occurring BoNT variant sequences. Recent progresses in genomic sequencing revealed that there are a growing number of subtype sequences within each major serotype of BoNTs¹⁸. For instance, BoNT/A currently contains at least 8 subtypes (A1-A8), with a total of 37 distinct sequences (https://bontbase.org/). There are as much as 15% differences at protein levels among these subtypes. These naturally-occurred subtype sequences offer a vast resource to guide mutagenesis. For example, residues that are conserved across majority of variant (consensus sequence) are likely critical for proper folding and function the molecular, and thus cannot be altered¹⁹. On the other hand, residues that are not conserved across variants are likely the ones that can be mutated without affecting the property of the toxin. Both the structure information and consensus sequences were utilized to design and evaluate “de-lysine” K to R mutations in BoNT/A-LC.

Design of K to R Mutations of LC/A1

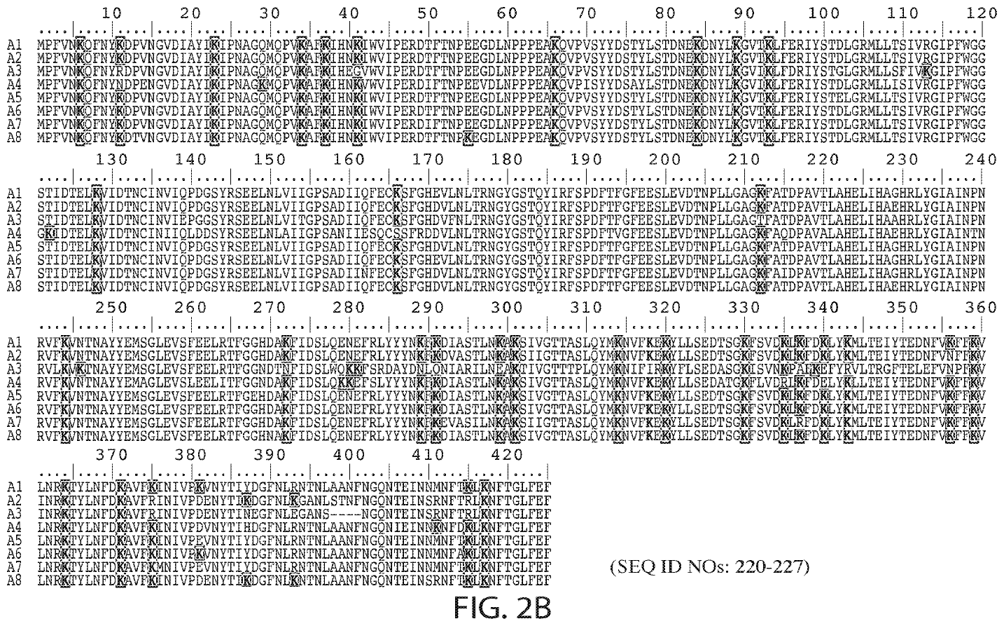

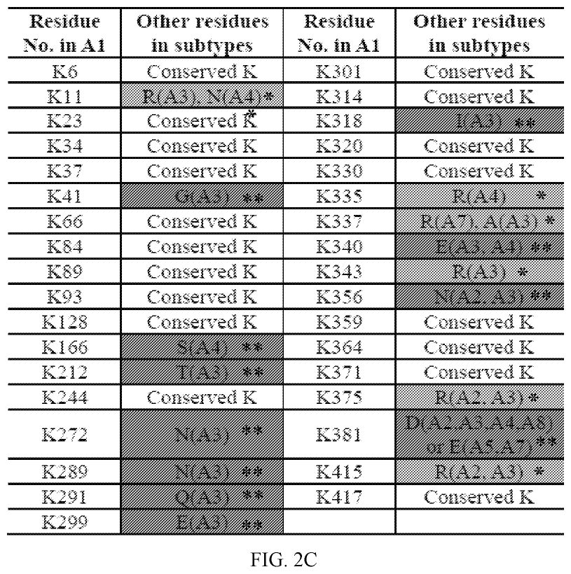

The crystal structure of BoNT/A1-LC (LC/A1) revealed that it has 35 lysines including 33 on its surface and two (K166 and K320) located inside the molecule (FIG. 2A). To avoid any detrimental effect caused by the K-to-R mutation, the LC sequence of A1 were aligned to A8 (FIG. 2B). There are 18 lysines on A1-LC that are conserved across subtypes, suggesting that they are critical for proper folding and/or function (FIG. 2C). Six lysines in A1 (K11, K335, K337, K343, K375 and K415) are already altered to R in some subtype sequences (FIG. 2C), and the remaining 10 non-conserved surface lysines (K41, K212, K272, K289, K291, K299, K318, K340, K356 and K381) are replaced by non-charged or negatively charged amino acids in other subtypes (FIG. 2C). Based on this consensus sequence analysis, it was decided to focus on these 16 non-conserved lysines to perform K to R mutagenesis on the standard BoNT/A1 sequence.

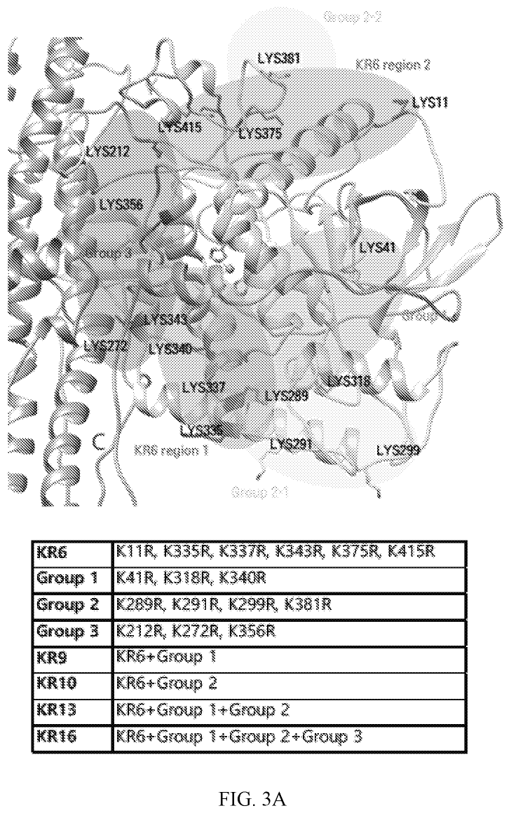

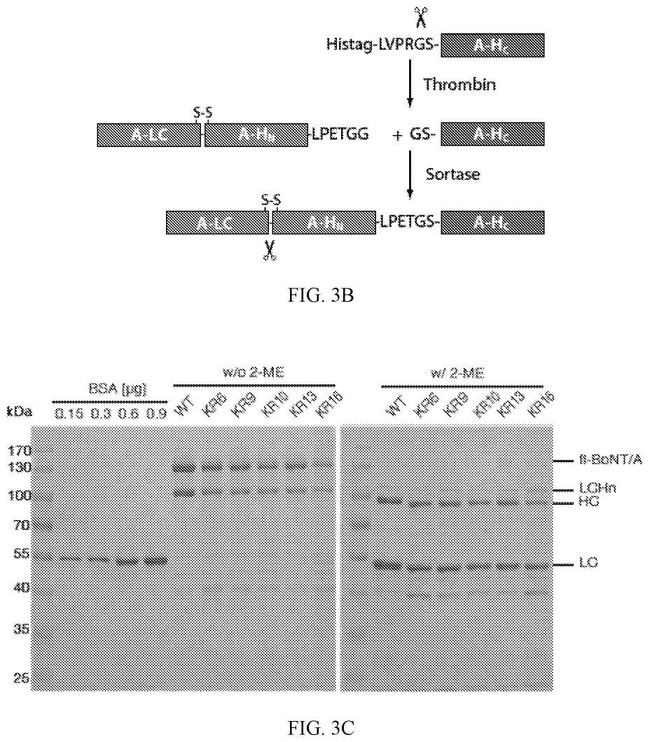

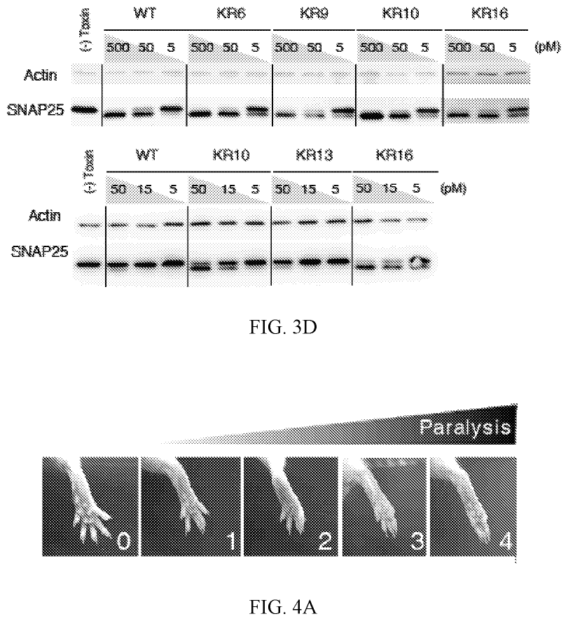

To approach the mutagenesis systematically, these 16 mutation sites were divided in four groups (FIG. 3A). The first group (Group 0), designated KR6, contains 6 mutation sites (11, 335, 337, 343, 375, 415) that are already R in other BoNT/A subtypes. Group 1 contain mutations at residues 41, 318, and 340, which are located near or at the binding interface with the toxin substrate SNAP-25²⁰. Group 2 contains mutations at 289, 291, 299, and 381, which are residues that are away from SNAP-25 binding region. Group 3 contains mutations at 212, 272, and 356, which are located between LC and H_{N}. Combinational mutations were designed as KR9 (KR6+group 1); KR10 (KR6+group 2); KR13 (KR6+groups 1 and 2); and KR16 (KR6+groups 1, 2, and 3).

Experimental Validation of BoNT/A1 Mutations

These selected mutations were next evaluated to determine if the mutations enhance the overall efficacy of BoNT/A1. To ensure biosafety, cDNA sequences that encode full-length toxin or develop any live organisms that may harbor the full toxin gene were not produced. Instead, a previously developed sortase-mediated ligation method was used (FIG. 3B)^{21,22}. This method relies on producing two separated pieces of BoNTs: one is the LC-H_{N} fragment with a sortase recognition site at the C-terminus; the other piece is the H_{C} with a free glycine at its N-terminus. Each piece alone is not toxic. The two proteins together can then be ligated biochemically by adding sortase (a bacterial transpeptidase), which recognizes the sortase tag (LPETG) and ligates it to the glycine at the N-terminal of H_{C}, thus producing a full-length toxin protein, without ever having the coding sequence for the full-length toxin. In this way, the amount of toxins produced in the test tube can be precisely controlled to ensure that the total amount is below the exempted amount for BoNTs (<1 mg).

WT and KR mutants of LCH_{N}/A1 were produced in E. coli, and then ligated to H_{C}/A1, generating full length toxins at ˜150 kDa (FIG. 3C). The linker region between the LC and H_{N} needs to be separated by proteases in order to be able to release LC upon its entry into the cytosol. This process is known as “activation” and activated LC-H_{N} remain connected by an inter-chain disulfide bond before reaching the cytosol. To facilitate the activation step, a thrombin cleavage site was introduced between LC and H_{N}. Treatment of thrombin resulted in almost complete activation of these ligated toxins (FIG. 3C).

The activity of WT versus KR mutants were compared in cultured rat cortical neurons. Cultured rat cortical neurons were exposed to WT, KR6, KR9, KR10, and KR16 full-length BoNT/A1 at three different concentrations (500, 50, and 5 pM) for 12 hours. In addition, WT, KR10, KR13, and KR16 were also examined at 50, 15, and 5 pM concentrations (FIG. 3D, lower panel). Entry of the toxin will result in cleavage of its substrate, a protein named SNAP-25, in neurons, which can be detected by immunoblot of cell lysates. The cleaved SNAP-25 can still be detected by the antibody used, showing up as a band with smaller molecular weight than the intact SNAP-25 (FIG. 3D). Compared with WT toxin, KR6, KR9, KR10, and KR16 showed a slightly enhanced cleavage of SNAP-25 at 5 pM concentration, suggesting that these KR mutations have a higher level of efficacy on cortical neurons than WT toxin. The enhanced cleavage of SNAP-25 is further confirmed for KR10 and KR16 at both 50 and 15 pM as well (FIG. 3D, lower panel).

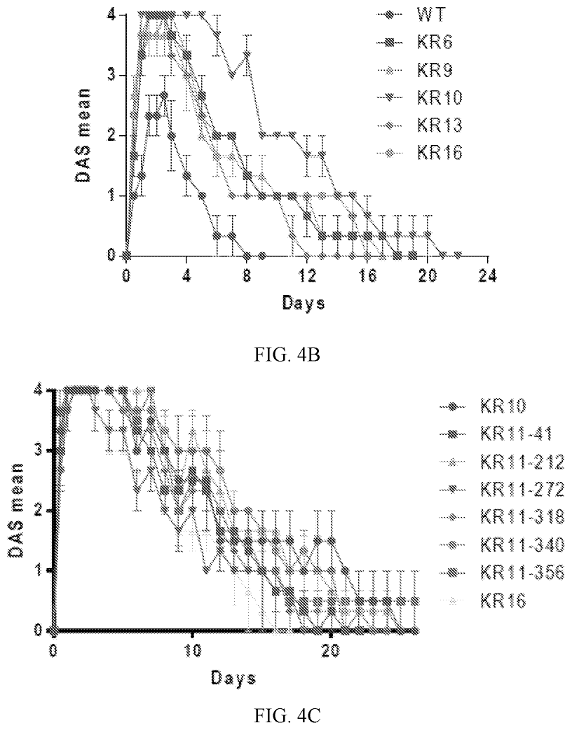

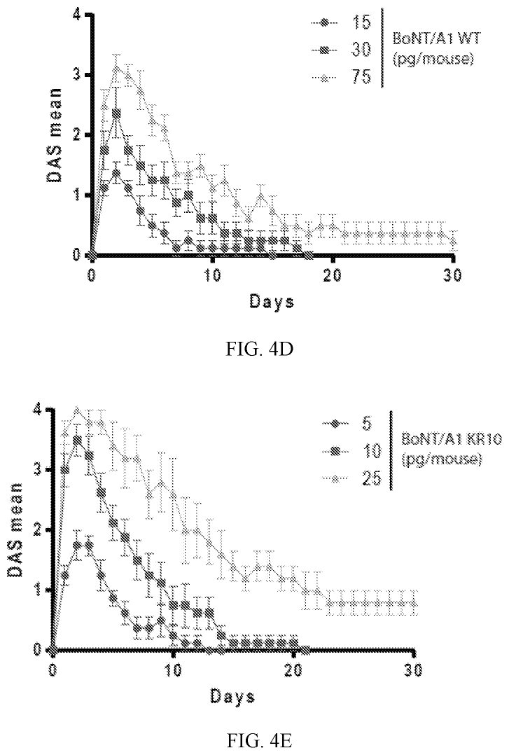

All these ligated toxins were evaluated and compared in vivo for their efficacy in inducing local muscle paralysis using a well-established mouse Digit Abduction Score (DAS) assay²³. A tiny amount of ligated toxin was injected into the hind leg of mice, and paralysis of this leg leads to loss of ability to spread the toe during startle response. The degree of toe spread reflects the degree of muscle paralysis, and can be quantified on the scale of 0-4, with 0 represents no paralysis and 4 represents the severest paralysis (FIG. 4A). The equal amount of WT and KR mutant toxins were injected and the DAS scores were recorded. As shown in FIG. 4B, all KR mutants showed higher DAS scores (reflecting potency) than WT toxin. KR6, KR9, KR13, and KR16 performed similarly, whereas KR10 is clearly the best with the longest duration of score 4. The therapeutic duration (TD) can be defined as the time that the DAS scores staying above 2. Compare to WT toxin with TD ˜3 days, the same dose of KR6, KR9, KR13, and KR16 showed TD of ˜6 days, and KR10 showed TD of ˜11 days (FIG. 4B), more than 3-fold extension of TD. Further adding additional KR mutations from groups 1 and 3 to KR10, which generates a series of KR11, did not improve the in vivo activity, with KR10 still the most potent modified BoNT (FIG. 4C).

To further compare WT and KR10 mutant toxins, three different doses of toxins were injected (15, 30, and 75 pg for WT, and 5, 10 and 25 pg for KR10). Both toxins elicited a dose-dependent response in the DAS mean (FIGS. 4D and 4E). KR10 clearly showed higher potency at all three doses than WT toxin. The increase in potency is >3-fold. For example, 10 pg of KR10 induced a higher DAS score than 30 pg of WT toxin, and 25 pg KR10 also induced higher DAS score than 75 pg of WT toxin. The DAS score for 5 pg KR10 is slightly lower than 30 pg of WT toxin, suggesting that the overall enhancement in potency is between 3 to 6-fold over WT toxin.

A series of additional mutations were generated and tested as listed in Table 1. Mutations that include conserved lysine sites are often not soluble (Table 1).

Combination with Other Mutations in H_{C} and in the Context of Chimeric Toxins

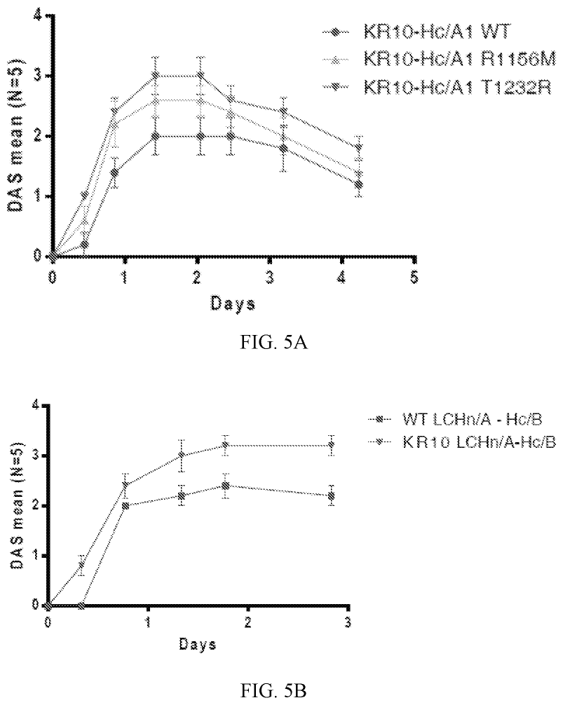

It was explored whether the benefit of KR10 mutations can be combined with additional mutations in receptor binding (H_{C}) domain to create a BoNT/A1 mutant with further enhanced activity. A series of point mutations were previously identified within H_{C}/A that enhance its binding to neurons (U.S. Application 63/128,758, which is incorporated by reference herein in its entirety). Two representative H_{C} mutations, R1156M and T1232R, were selected and ligated to the KR10-LC-H_{N}. 5 pg of the full-length ligated toxin were injected in DAS assays. Both KR10-R1156M and KR10-T1232R showed higher level of potency than KR10 mutant alone, with KR10-T1232R performing best (FIG. 5A). These results demonstrate that the benefit of KR10 and mutations in H_{C}/A can be combined to generate the better BoNT/A mutants with further enhanced potency than mutations in individual domains.

Finally, it was evaluated whether the benefit of KR10 can be maintained in the context of chimeric toxins. WT or KR10 LCH_{N} of BoNT/A1 were ligated with the WT BoNT/B1 H_{C} and compared using DAS assays. As shown in FIG. 5B, KR10-AB could reach score 3, while WT AB only reached score 2, suggesting the enhanced efficacy from KR10 is maintained in the context of chimeric toxins and demonstrate that KR10 LC/A1 can be utilized to construct chimeric toxins.

Together, these results demonstrate that changing lysines into arginines on the surface of A1-LC made the full-length toxin more potent in vitro and in vivo. These mutations can be used to create a new generation of BoNT/A with improved efficacy compared to the natural BoNT/A. These mutations can also be introduced to other, potentially superior A subtypes (e.g. A2 and A6) in addition to A1 to create novel engineered toxin with improved potency, because they share most of surface lysines on the corresponding positions. The light chain of BoNT/A containing these mutations can also be utilized to create chimeric toxins, such as BoNT/AB.

Materials and Methods

Antibodies and materials: Mouse monoclonal antibody for SNAP25 (C171.2) was generously provided by E. Chapman (Madison, WI) and are available from Synaptic Systems (Goettingen, Germany). Mouse monoclonal antibody for actin was purchased from Sigma (AC-15).

cDNA and constructs: The cDNAs encoding BoNT/A1-LC (residues 1-441) with KR6 and KR16 mutations were synthesized by Genewiz Inc. and its codon has been optimized for expression in E. coli. The DNAs encoding BoNT/A1-KR mutants were subcloned into pET28a vector containing H_{N}/A1 with both a LPETGG (SEQ ID NO: 130) tag for sortase ligation and a His6 tag for purification at the C-terminus of the H_{N}. A thrombin cleavage site was introduced between LC and H_{N} sequences. Other KR mutants were generated by site-directed mutagenesis using KR6 or KR16 as PCR templates. DNA encoding BoNT/A1-H_{C} was obtained from a previous study²⁶, but the non-essential HA tag was removed from the construct. All constructs were verified by sequencing.

Protein expression, purification and activation: WT and mutants of BoNT/A1-LCH_{N} were expressed as His6 tagged recombinant proteins in E. coli BL21(DE3) strain with the induction temperature at 18° C. overnight in the autoinduction medium (Formedium, Ltd.). His6-fusion proteins were purified using Ni-NTA bead charged column. Briefly, bacterial lysates were incubated with Ni-NTA beads, followed by flow-through of washing buffer (40 mM imidazole, TBS, pH7.5) and elution buffer (100-500 mM imidazole, TBS, pH7.5). Eluted proteins were desalted and digested with thrombin Sepharose bead (BioVision, Inc.). Inactivated proteins were removed using prewashed Ni-NTA beads, and supernatant (activated proteins) was concentrated, quantified and frozen (−80° C.) until use.

Sortase-mediated ligation: 4 μM of substrate proteins, LC-H_{N} and H_{C} were premixed with TBS, pH7.5 containing 10 mM CaCl₂. 0.5 μM of recombinant sortase was added and incubated for 40 min at room temperature. Prewashed Ni-NTA beads were loaded to remove His-tagged sortase and unligated substrate proteins. Supernatant was only activated using 25 U/ml thrombin (Millipore) for 40 min at room temperature. Some of ligated products were analyzed on the SDS-PAGE gel with or without 2-mercaptoethanol to confirm ligation and activation. Full-length toxins were quantified based on protein density on the gel using BSA as a standard (0.15-0.9 μg).

Neuron culture and SNAP25 cleavage assays: Rat cortical neurons were prepared from E18-19 embryos as described previously²⁷. The quantified full-length toxins were mixed with medium, where the neurons were cultured. After desired incubation time, cells were washed and subjected to immunoblotting analysis.

Digit Abduction Score (DAS) assay. All procedures were conducted in accordance with the guidelines approved by the Institute Animal Care and Use Committee (IACUC) at Boston Children's Hospital (#3030). Briefly, full-length BoNT/A1-WT or KR mutants generated by sortase-mediated ligation and thrombin activation were diluted with 0.2% of gelatin-containing phosphate buffer. Mice (CD-1 strain, female, purchased from Envigo, 5-6 weeks old, 21-25 g) were anesthetized with isoflurane (3-4%) and injected with the prepared full-length toxin (10 μL) using a 30-gauge needle attached to a sterile Hamilton syringe, into the gastrocnemius muscles of the right hind limb. Muscle paralysis and the spread of hind paw in response to a startle stimulus were observed according to FIG. 4A.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Also, while embodiments herein are described with respect to controlling flow of air in the device to move the suction tip, it should be appreciated that the other gases in the surgical field may flow in the device to cause the desired movement of the suction tip. In other words, the use of the terms “air” and “airflow” should not be considered as being limited to air, as other gases in the environment/surgical site may be present.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

TABLE 1
All the tested KR and non-K-to-R mutants

| | | |
|---|---|---|
| Construct name | Sequence information | Full-length toxin activity |

K-to-R mutants

| | | |
|---|---|---|
| KR6 | K11R, K335R, K337R, K343R, K375R, K415R | ** |
| KR9 | KR6 + K41R, K318R, K340R | ** |
| KR10 | KR6 + K289R, K291R, K299R, K381R | ** |
| KR11-41 | KR10 + K41R | ** |
| KR11-212 | KR10 + K212R | ** |
| KR11-272 | KR10 + K272R | ** |
| KR11-318 | KR10 + K318R | ** |
| KR11-340 | KR10 + K340R | ** |
| KR11-356 | KR10 + K356R | ** |
| KR13 | KR10 + K41R, K318R, K340R | ** |
| KR15 | KR16 lacking K272R | ** |
| KR16 | KR13 + K212R, K272R, K356R | ** |
| KR26 | KR33 + R41K, R66K, R84K, R93K, R314K, | Rarely expressed |
| | R340K, R371K | |
| KR28 | KR33 + R66K, R84K, R93K, R314K, R371K | Rarely expressed |
| KR29 | KR33 + R41K, R84K, R93K, R371K | Rarely expressed |
| KR30 | KR33 + R84K, R93K, R371K | Rarely expressed |
| KR33 | All K to R mutations except K166 and K320 | Rarely expressed |

Non-K-to-R mutants

| | | |
|---|---|---|
| nKR-Y387R | Y387R | * |
| nKR-M411R | M411R | * |

Other A subtype KR mutants

| | | |
|---|---|---|
| A2-KR7 | K11R, K289R, K291R, K299R, K335R, K337R, | better than A2 WT |
| | K343R | |

similar in vivo activity compared to WT
** at least two times better in vivo toxicity compared to WT

TABLE 2
Amino Acid Sequences of BoNTs

| SEQ ID | | |
|---|---|---|
| NO: | Description | Sequence |
| 1 | BoNT/A1-WT | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 2 | BoNT/A1-K11R | MPFVNKQFNYRDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 3 | BoNT/A1-K41R | MPFVNKQFNYKDPVNGVDIAYIKIPNAGQMQPVKAFKIHNRIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 4 | BoNT/A1-K212R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GRFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 5 | BoNT/A1-K272R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDARFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 6 | BoNT/A1-K289R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 7 | BoNT/A1-K291R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFRDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 8 | BoNT/A1-K299R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNRAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 9 | BoNT/A1-K318R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 10 | BoNT/A1-K335R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDRLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| | | MPFVNKQFNYKDPVNGVDIAYIKIPNAGQMQPVKAFKIHNKIWVIPERDT |
| 11 | BoNT/A1-K337R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLRFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 12 | BoNT/A1-K340R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDRLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 13 | BoNT/A1-K343R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYRMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 14 | BoNT/A1-K356R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVRFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 15 | BoNT/A1-K375R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFRINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 16 | BoNT/A1-K381R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPRVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 17 | BoNT/A1-Y387R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIRDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 18 | BoNT/A1-M411R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNRNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 19 | BoNT/A1-K415R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTRLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 20 | BoNT/A1-Group 0 | MPFVNKQFNYRDPVNGVDIAYIKIPNAGQMQPVKAFKIHNKIWVIPERDT |
| | (K11R, K335R, K337R, | FTNPEEGDLNPPPEAKQVPVSYYDSTYLSTDNEKDNYLKGVTKLFERIYSTDL |
| | K343R, K375R, K415R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDRLRFDKLYRMLTEIYTEDNFVKFFKVLNRKTYL |
| | | NFDKAVFRINIVPKVNYTIYDGFNLRNTNLAANFNGQNTEINNMNFTRLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 21 | BoNT/A1-Group 1 | MPFVNKQFNYKDPVNGVDIAYIKIPNAGQMQPVKAFKIHNRIWVIPERDT |
| | (K41R, K318R, K340R) | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDKLKFDRLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 22 | BoNT/A1-Group 2 | MPFVNKQFNYKDPVNGVDIAYIKIPNAGQMQPVKAFKIHNKIWVIPERDT |
| | (K289R, K291R, | FTNPEEGDLNPPPEAKQVPVSYYDSTYLSTDNEKDNYLKGVTKLFERIYSTDL |
| | K299R, K381R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDKLKFDKLYKMLTEIYTEDNFVKFFKVLNRKTYL |
| | | NFDKAVFKINIVPRVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 23 | BoNT/A1-Group 3 | MPFVNKQFNYKDPVNGVDIAYIKIPNAGQMQPVKAFKIHNKIWVIPERDT |
| | (K212R, K272R, | FTNPEEGDLNPPPEAKQVPVSYYDSTYLSTDNEKDNYLKGVTKLFERIYSTDL |
| | K356R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GRFATDPAVTLAHELIHAGHRLYGIAINPNRVFKVNTNAYYEMSGLEVSFEE |
| | | LRTFGGHDARFIDSLQENEFRLYYYNKFKDIASTLNKAKSIVGTTASLQYMK |
| | | NVFKEKYLLSEDTSGKFSVDKLKFDKLYKMLTEIYTEDNFVRFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 24 | BoNT/A1-KR9 (K11R, | MPFVNKQFNYRDPVNGVDIAYIKIPNAGQMQPVKAFKIHNRIWVIPERDT |
| | K41R, K318R, K335R, | FTNPEEGDLNPPPEAKQVPVSYYDSTYLSTDNEKDNYLKGVTKLFERIYSTDL |
| | K337R, K340R, K343R, | GRMLLTSIVRGIPFWGGSTIDTELKVIDTNCINVIQPDGSYRSEELNLVIIGPS |
| | K375R, K415R) | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDRLRFDRLYRMLTEIYTEDNFVKFFKVLNRKTYL |
| | | NFDKAVFRINIVPKVNYTIYDGFNLRNTNLAANFNGQNTEINNMNFTRLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKNIINTSILNLRYESNHLIDLSRYASKI |
| | | NIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 25 | BoNT/A1-KR10 (K11R, | MPFVNKQFNYRDPVNGVDIAYIKIPNAGQMQPVKAFKIHNKIWVIPERDT |
| | K289R, K291R, K299R, | FTNPEEGDLNPPPEAKQVPVSYYDSTYLSTDNEKDNYLKGVTKLFERIYSTDL |
| | K335R, K337R, K343R, | GRMLLTSIVRGIPFWGGSTIDTELKVIDTNCINVIQPDGSYRSEELNLVIIGPS |
| | K375R, K381R, K415R) | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 26 | BoNT/A1-KR11-41 | MPFVNKQFNYRDPVNGVDIAYIKIPNAGQMQPVKAFKIHNRIWVIPERDT |
| | (K11R, K41R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 27 | BoNT/A1-KR11-212 | MPFVNKQFNYRDPVNGVDIAYIKIPNAGQMQPVKAFKIHNKIWVIPERDT |
| | (K11R, K212R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GRFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 28 | BoNT/A1-KR11-272 | MPFVNKQFNYRDPVNGVDIAYIKIPNAGQMQPVKAFKIHNKIWVIPERDT |
| | (K11R, K272R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDARFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 29 | BoNT/A1-KR11-318 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K318R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFREKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 30 | BoNT/A1-KR11-340 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K340R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDRLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 31 | BoNT/A1-KR11-356 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K343R, K356R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 32 | BoNT/A1-KR13 (K11R, | MPFVNKQFNYRDPVNGVDIAYIKIPNAGQMQPVKAFKIHNRIWVIPERDT |
| | K41R, K289R, K291R, | FTNPEEGDLNPPPEAKQVPVSYYDSTYLSTDNEKDNYLKGVTKLFERIYSTDL |
| | K299R, K318R, K335R, | GRMLLTSIVRGIPFWGGSTIDTELKVIDTNCINVIQPDGSYRSEELNLVIIGPS |
| | K337R, K340R, K343R, | ADIIQFECKSFGHEVLNLTRNGYGSTQYIRFSPDFTFGFEESLEVDTNPLLGA |
| | K375R, K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFREKYLLSEDTSGKFSVDRLRFDRLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 33 | BoNT/A1-KR15 (K11R, | MPFVNKQFNYRDPVNGVDIAYIKIPNAGQMQPVKAFKIHNRIWVIPERDT |
| | K41R, K212R, K289R, | FTNPEEGDLNPPPEAKQVPVSYYDSTYLSTDNEKDNYLKGVTKLFERIYSTDL |
| | K291R, K299R, K318R, | GRMLLTSIVRGIPFWGGSTIDTELKVIDTNCINVIQPDGSYRSEELNLVIIGPS |
| | K335R, K337R, K340R, | ADIIQFECKSFGHEVLNLTRNGYGSTQYIRFSPDFTFGFEESLEVDTNPLLGA |
| | K343R, K356R, K375R, | GRFATDPAVTLAHELIHAGHRLYGIAINPNRVFKVNTNAYYEMSGLEVSFEE |
| | K381R, K415R) | LRTFGGHDAKFIDSLQENEFRLYYYNRFRDIASTLNRAKSIVGTTASLQYMK |
| | | NVFREKYLLSEDTSGKFSVDRLRFDRLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 34 | BoNT/A1-KR16 (K11R, | MPFVNKQFNYRDPVNGVDIAYIKIPNAGQMQPVKAFKIHNRIWVIPERDT |
| | K41R, K212R, K272R, | FTNPEEGDLNPPPEAKQVPVSYYDSTYLSTDNEKDNYLKGVTKLFERIYSTDL |
| | K289R, K291R, K299R, | GRMLLTSIVRGIPFWGGSTIDTELKVIDTNCINVIQPDGSYRSEELNLVIIGPS |
| | K318R, K335R, K337R, | ADIIQFECKSFGHEVLNLTRNGYGSTQYIRFSPDFTFGFEESLEVDTNPLLGA |
| | K340R, K343R, K356R, | GRFATDPAVTLAHELIHAGHRLYGIAINPNRVFKVNTNAYYEMSGLEVSFEE |
| | K375R, K381R, K415R) | LRTFGGHDARFIDSLQENEFRLYYYNRFRDIASTLNRAKSIVGTTASLQYMK |
| | | NVFREKYLLSEDTSGKFSVDRLRFDRLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 35 | BoNT/A2-WT | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 36 | BoNT/A2-K11R | MPFVNKQFNYRDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 37 | BoNT/A2-K289R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNRFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 38 | BoNT/A2-K291R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFRDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 39 | BoNT/A2-K299R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNRAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 40 | BoNT/A2-K335R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDRLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 41 | BoNT/A2-K337R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLRFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 42 | BoNT/A2-K343R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 43 | BoNT/A2-KR7 (K11R, | MPFVNKQFNYRDPVNGVDIAYIKIPNAGQMQPVKAFKIHNKIWVIPERDT |
| | K289R, K291R, K299R, | FTNPEEGDLNPPPEAKQVPVSYYDSTYLSTDNEKDNYLKGVTKLFERIYSTDL |
| | K335R, K337R, K343R) | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNRFRDVASTLNRAKSIIGTTASLQYMK |
| | | NVFKEKYLLSEDTSGKFSVDRLRFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKNIVNTSILSIVYKKDDLIDLSRY |
| | | GAKINIGDRVYYDSIDKNQIKLINLESSTIEVILKNAIVYNSMYENFSTSFWIKI |
| | | PKYFSKINLNNEYTIINCIENNSGWKVSLNYGEIIWTLQDNKQNIQRVVFKYS |
| | | QMVNISDYINRWIFVTITNNRLTKSKIYINGRLIDQKPISNLGNIHASNKIMF |
| | | KLDGCRDPRRYIMIKYFNLFDKELNEKEIKDLYDSQSNSGILKDFWGNYLQY |
| | | DKPYYMLNLFDPNKYVDVNNIGIRGYMYLKGPRGSVVTTNIYLNSTLYEGT |
| | | KFIIKKYASGNEDNIVRNNDRVYINVVVKNKEYRLATNASQAGVEKILSALEI |
| | | PDVGNLSQVVVMKSKDDQGIRNKCKMNLQDNNGNDIGFIGFHLYDNIAK |
| | | LVASNWYNRQVGKASRTFGCSWEFIPVDDGWGESSL |
| 44 | LC-Hn BoNT/A1-WT | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 45 | LC-Hn BoNT/A1-K11R | MPFVNKQFNYRDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 46 | LC-Hn BoNT/A1-K41R | MPFVNKQFNYKDPVNGVDIAYIKIPNAGQMQPVKAFKIHNRIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 47 | LC-Hn BoNT/A1- | MPFVNKQFNYKDPVNGVDIAYIKIPNAGQMQPVKAFKIHNKIWVIPERDT |
| | K212R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GRFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 48 | LC-Hn BoNT/A1- | MPFVNKQFNYKDPVNGVDIAYIKIPNAGQMQPVKAFKIHNKIWVIPERDT |
| | K272R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDARFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 49 | LC-Hn BoNT/A1- | MPFVNKQFNYKDPVNGVDIAYIKIPNAGQMQPVKAFKIHNKIWVIPERDT |
| | K289R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 50 | LC-Hn BoNT/A1- | MPFVNKQFNYKDPVNGVDIAYIKIPNAGQMQPVKAFKIHNKIWVIPERDT |
| | K291R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFRDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 51 | LC-Hn BoNT/A1- | MPFVNKQFNYKDPVNGVDIAYIKIPNAGQMQPVKAFKIHNKIWVIPERDT |
| | K299R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNRAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 52 | LC-Hn BoNT/A1- | MPFVNKQFNYKDPVNGVDIAYIKIPNAGQMQPVKAFKIHNKIWVIPERDT |
| | K318R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 53 | LC-Hn BoNT/A1- | MPFVNKQFNYKDPVNGVDIAYIKIPNAGQMQPVKAFKIHNKIWVIPERDT |
| | K335R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDRLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 54 | LC-Hn BoNT/A1- | MPFVNKQFNYKDPVNGVDIAYIKIPNAGQMQPVKAFKIHNKIWVIPERDT |
| | K337R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLRFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 55 | LC-Hn BoNT/A1- | MPFVNKQFNYKDPVNGVDIAYIKIPNAGQMQPVKAFKIHNKIWVIPERDT |
| | K340R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDRLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 56 | LC-Hn BoNT/A1- | MPFVNKQFNYKDPVNGVDIAYIKIPNAGQMQPVKAFKIHNKIWVIPERDT |
| | K343R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYRMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 57 | LC-Hn BoNT/A1- | MPFVNKQFNYKDPVNGVDIAYIKIPNAGQMQPVKAFKIHNKIWVIPERDT |
| | K356R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVRFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 58 | LC-Hn BoNT/A1- | MPFVNKQFNYKDPVNGVDIAYIKIPNAGQMQPVKAFKIHNKIWVIPERDT |
| | K375R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFRINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 59 | LC-Hn BoNT/A1- | MPFVNKQFNYKDPVNGVDIAYIKIPNAGQMQPVKAFKIHNKIWVIPERDT |
| | K381R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPRVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 60 | LC-Hn BoNT/A1-Y387R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIRDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 61 | LC-Hn BoNT/A1- | MPFVNKQFNYKDPVNGVDIAYIKIPNAGQMQPVKAFKIHNKIWVIPERDT |
| | M411R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNRNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 62 | LC-Hn BoNT/A1- | MPFVNKQFNYKDPVNGVDIAYIKIPNAGQMQPVKAFKIHNKIWVIPERDT |
| | K415R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTRLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 63 | LC-Hn BoNT/A1-Group | MPFVNKQFNYRDPVNGVDIAYIKIPNAGQMQPVKAFKIHNKIWVIPERDT |
| | 0 (K11R, K335R, | FTNPEEGDLNPPPEAKQVPVSYYDSTYLSTDNEKDNYLKGVTKLFERIYSTDL |
| | K337R, K343R, K375R, | GRMLLTSIVRGIPFWGGSTIDTELKVIDTNCINVIQPDGSYRSEELNLVIIGPS |
| | K415R) | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDRLRFDKLYRMLTEIYTEDNFVKFFKVLNRKTYL |
| | | NFDKAVFRINIVPKVNYTIYDGFNLRNTNLAANFNGQNTEINNMNFTRLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 64 | LC-Hn BoNT/A1-Group | MPFVNKQFNYKDPVNGVDIAYIKIPNAGQMQPVKAFKIHNRIWVIPERDT |
| | 1 (K41R, K318R, | FTNPEEGDLNPPPEAKQVPVSYYDSTYLSTDNEKDNYLKGVTKLFERIYSTDL |
| | K340R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDKLKFDRLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 65 | LC-Hn BoNT/A1-Group | MPFVNKQFNYKDPVNGVDIAYIKIPNAGQMQPVKAFKIHNKIWVIPERDT |
| | 2 (K289R, K291R, | FTNPEEGDLNPPPEAKQVPVSYYDSTYLSTDNEKDNYLKGVTKLFERIYSTDL |
| | K299R, K381R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDKLKFDKLYKMLTEIYTEDNFVKFFKVLNRKTYL |
| | | NFDKAVFKINIVPRVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 66 | LC-Hn BoNT/A1-Group | MPFVNKQFNYKDPVNGVDIAYIKIPNAGQMQPVKAFKIHNKIWVIPERDT |
| | 3 (K212R, K272R, | FTNPEEGDLNPPPEAKQVPVSYYDSTYLSTDNEKDNYLKGVTKLFERIYSTDL |
| | K356R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GRFATDPAVTLAHELIHAGHRLYGIAINPNRVFKVNTNAYYEMSGLEVSFEE |
| | | LRTFGGHDARFIDSLQENEFRLYYYNKFKDIASTLNKAKSIVGTTASLQYMK |
| | | NVFKEKYLLSEDTSGKFSVDKLKFDKLYKMLTEIYTEDNFVRFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 67 | LC-Hn BoNT/A1-KR9 | MPFVNKQFNYRDPVNGVDIAYIKIPNAGQMQPVKAFKIHNRIWVIPERDT |
| | (K11R, K41R, K318R, | FTNPEEGDLNPPPEAKQVPVSYYDSTYLSTDNEKDNYLKGVTKLFERIYSTDL |
| | K335R, K337R, K340R, | GRMLLTSIVRGIPFWGGSTIDTELKVIDTNCINVIQPDGSYRSEELNLVIIGPS |
| | K343R, K375R, K415R) | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDRLRFDRLYRMLTEIYTEDNFVKFFKVLNRKTYL |
| | | NFDKAVFRINIVPKVNYTIYDGFNLRNTNLAANFNGQNTEINNMNFTRLKN |
| | | FTGLFEFYKLLCVRGIITSKTKSLDKGYNKALNDLCIKVNNWDLFFSPSEDNF |
| | | TNDLNKGEEITSDTNIEAAEENISLDLIQQYYLTFNFDNEPENISIENLSSDIIG |
| | | QLELMPNIERFPNGKKYELDKYTMFHYLRAQEFEHGKSRIALTNSVNEALLN |
| | | PSRVYTFFSSDYVKKVNKATEAAMFLGWVEQLVYDFTDETSEVSTTDKIADI |
| | | TIIIPYIGPALNIGNMLYKDDFVGALIFSGAVILLEFIPEIAIPVLGTFALVSYIAN |
| | | KVLTVQTIDNALSKRNEKWDEVYKYIVTNWLAKVNTQIDLIRKKMKEALEN |
| | | QAEATKAIINYQYNQYTEEEKNNINFNIDDLSSKLNESINKAMININKFLNQC |
| | | SVSYLMNSMIPYGVKRLEDFDASLKDALLKYIYDNRGTLIGQVDRLKDKVN |
| | | NTLSTDIPFQLSKYVDNQRLLSTFTEYIKN |
| 68 | LC-Hn BoNT/A1-KR10 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K343R, K375R, K381R, | ADIIQFECKSFGHEVLNLTRNGYGSTQYIRFSPDFTFGFEESLEVDTNPLLGA |
| | K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 69 | LC-Hn BoNT/A1-KR11- | MPFVNKQFNYRDPVNGVDIAYIKIPNAGQMQPVKAFKIHNRIWVIPERDT |
| | 41 (K11R, K41R, | FTNPEEGDLNPPPEAKQVPVSYYDSTYLSTDNEKDNYLKGVTKLFERIYSTDL |
| | K289R, K291R, K299R, | GRMLLTSIVRGIPFWGGSTIDTELKVIDTNCINVIQPDGSYRSEELNLVIIGPS |
| | K335R, K337R, K343R, | ADIIQFECKSFGHEVLNLTRNGYGSTQYIRFSPDFTFGFEESLEVDTNPLLGA |
| | K375R, K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 70 | LC-Hn BoNT/A1-KR11- | MPFVNKQFNYRDPVNGVDIAYIKIPNAGQMQPVKAFKIHNKIWVIPERDT |
| | 212 (K11R, K212R, | FTNPEEGDLNPPPEAKQVPVSYYDSTYLSTDNEKDNYLKGVTKLFERIYSTDL |
| | K289R, K291R, K299R, | GRMLLTSIVRGIPFWGGSTIDTELKVIDTNCINVIQPDGSYRSEELNLVIIGPS |
| | K335R, K337R, K343R, | ADIIQFECKSFGHEVLNLTRNGYGSTQYIRFSPDFTFGFEESLEVDTNPLLGA |
| | K375R, K381R, K415R) | GRFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 71 | LC-Hn BoNT/A1-KR11- | MPFVNKQFNYRDPVNGVDIAYIKIPNAGQMQPVKAFKIHNKIWVIPERDT |
| | 272 (K11R, K272R, | FTNPEEGDLNPPPEAKQVPVSYYDSTYLSTDNEKDNYLKGVTKLFERIYSTDL |
| | K289R, K291R, K299R, | GRMLLTSIVRGIPFWGGSTIDTELKVIDTNCINVIQPDGSYRSEELNLVIIGPS |
| | K335R, K337R, K343R, | ADIIQFECKSFGHEVLNLTRNGYGSTQYIRFSPDFTFGFEESLEVDTNPLLGA |
| | K375R, K381R, K415R) | GKFATDPAVTLAHELIHAGHRLYGIAINPNRVFKVNTNAYYEMSGLEVSFEE |
| | | LRTFGGHDARFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 72 | LC-Hn BoNT/A1-KR11- | MPFVNKQFNYRDPVNGVDIAYIKIPNAGQMQPVKAFKIHNKIWVIPERDT |
| | 318 (K11R, K289R, | FTNPEEGDLNPPPEAKQVPVSYYDSTYLSTDNEKDNYLKGVTKLFERIYSTDL |
| | K291R, K299R, K318R, | GRMLLTSIVRGIPFWGGSTIDTELKVIDTNCINVIQPDGSYRSEELNLVIIGPS |
| | K335R, K337R, K343R, | ADIIQFECKSFGHEVLNLTRNGYGSTQYIRFSPDFTFGFEESLEVDTNPLLGA |
| | K375R, K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFREKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 73 | LC-Hn BoNT/A1-KR11- | MPFVNKQFNYRDPVNGVDIAYIKIPNAGQMQPVKAFKIHNKIWVIPERDT |
| | 340 (K11R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K340R, K343R, | ADIIQFECKSFGHEVLNLTRNGYGSTQYIRFSPDFTFGFEESLEVDTNPLLGA |
| | K375R, K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDRLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 74 | LC-Hn BoNT/A1-KR11- | MPFVNKQFNYRDPVNGVDIAYIKIPNAGQMQPVKAFKIHNKIWVIPERDT |
| | 356 (K11R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K356R, | ADIIQFECKSFGHEVLNLTRNGYGSTQYIRFSPDFTFGFEESLEVDTNPLLGA |
| | K375R, K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 75 | LC-Hn BoNT/A1-KR13 | MPFVNKQFNYRDPVNGVDIAYIKIPNAGQMQPVKAFKIHNRIWVIPERDT |
| | (K11R, K41R, K289R, | FTNPEEGDLNPPPEAKQVPVSYYDSTYLSTDNEKDNYLKGVTKLFERIYSTDL |
| | K291R, K299R, K318R, | GRMLLTSIVRGIPFWGGSTIDTELKVIDTNCINVIQPDGSYRSEELNLVIIGPS |
| | K335R, K337R, K340R, | ADIIQFECKSFGHEVLNLTRNGYGSTQYIRFSPDFTFGFEESLEVDTNPLLGA |
| | K343R, K375R, K381R, | GKFATDPAVTLAHELIHAGHRLYGIAINPNRVFKVNTNAYYEMSGLEVSFEE |
| | K415R) | LRTFGGHDAKFIDSLQENEFRLYYYNRFRDIASTLNRAKSIVGTTASLQYMK |
| | | NVFREKYLLSEDTSGKFSVDRLRFDRLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 76 | LC-Hn BoNT/A1-KR15 | MPFVNKQFNYRDPVNGVDIAYIKIPNAGQMQPVKAFKIHNRIWVIPERDT |
| | (K11R, K41R, K212R, | FTNPEEGDLNPPPEAKQVPVSYYDSTYLSTDNEKDNYLKGVTKLFERIYSTDL |
| | K289R, K291R, K299R, | GRMLLTSIVRGIPFWGGSTIDTELKVIDTNCINVIQPDGSYRSEELNLVIIGPS |
| | K318R, K335R, K337R, | ADIIQFECKSFGHEVLNLTRNGYGSTQYIRFSPDFTFGFEESLEVDTNPLLGA |
| | K340R, K343R, K356R, | GRFATDPAVTLAHELIHAGHRLYGIAINPNRVFKVNTNAYYEMSGLEVSFEE |
| | K375R, K381R, K415R) | LRTFGGHDAKFIDSLQENEFRLYYYNRFRDIASTLNRAKSIVGTTASLQYMK |
| | | NVFREKYLLSEDTSGKFSVDRLRFDRLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 77 | LC-Hn BoNT/A1-KR16 | MPFVNKQFNYRDPVNGVDIAYIKIPNAGQMQPVKAFKIHNRIWVIPERDT |
| | (K11R, K41R, K212R, | FTNPEEGDLNPPPEAKQVPVSYYDSTYLSTDNEKDNYLKGVTKLFERIYSTDL |
| | K272R, K289R, K291R, | GRMLLTSIVRGIPFWGGSTIDTELKVIDTNCINVIQPDGSYRSEELNLVIIGPS |
| | K299R, K318R, K335R, | ADIIQFECKSFGHEVLNLTRNGYGSTQYIRFSPDFTFGFEESLEVDTNPLLGA |
| | K337R, K340R, K343R, | GRFATDPAVTLAHELIHAGHRLYGIAINPNRVFKVNTNAYYEMSGLEVSFEE |
| | K356R, K375R, K381R, | LRTFGGHDARFIDSLQENEFRLYYYNRFRDIASTLNRAKSIVGTTASLQYMK |
| | K415R) | NVFREKYLLSEDTSGKFSVDRLRFDRLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKEAL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKN |
| 78 | LC-Hn BoNT/A2-WT | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 79 | LC-Hn BoNT/A2-K11R | MPFVNKQFNYRDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 80 | LC-Hn BoNT/A2- | MPFVNKQFNYKDPVNGVDIAYIKIPNAGQMQPVKAFKIHNKIWVIPERDT |
| | K289R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNRFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 81 | LC-Hn BoNT/A2- | MPFVNKQFNYKDPVNGVDIAYIKIPNAGQMQPVKAFKIHNKIWVIPERDT |
| | K291R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFRDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 82 | LC-Hn BoNT/A2- | MPFVNKQFNYKDPVNGVDIAYIKIPNAGQMQPVKAFKIHNKIWVIPERDT |
| | K299R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNRAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 83 | LC-Hn BoNT/A2- | MPFVNKQFNYKDPVNGVDIAYIKIPNAGQMQPVKAFKIHNKIWVIPERDT |
| | K335R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDRLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 84 | LC-Hn BoNT/A2- | MPFVNKQFNYKDPVNGVDIAYIKIPNAGQMQPVKAFKIHNKIWVIPERDT |
| | K337R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLRFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 85 | LC-Hn BoNT/A2- | MPFVNKQFNYKDPVNGVDIAYIKIPNAGQMQPVKAFKIHNKIWVIPERDT |
| | K343R | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 86 | LC-Hn BoNT/A2-KR7 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K343R) | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNRFRDVASTLNRAKSIIGTTASLQYMK |
| | | NVFKEKYLLSEDTSGKFSVDRLRFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKSLDEGYNKALNDLCIKVNNWDLFFSPSEDNFT |
| | | NDLDKVEEITADTNIEAAEENISLDLIQQYYLTFDFDNEPENISIENLSSDIIGQ |
| | | LEPMPNIERFPNGKKYELDKYTMFHYLRAQEFEHGDSRIILTNSAEEALLKP |
| | | NVAYTFFSSKYVKKINKAVEAFMFLNWAEELVYDFTDETNEVTTMDKIADI |
| | | TIIVPYIGPALNIGNMLSKGEFVEAIIFTGVVAMLEFIPEYALPVFGTFAIVSYI |
| | | ANKVLTVQTINNALSKRNEKWDEVYKYTVTNWLAKVNTQIDLIREKMKKA |
| | | LENQAEATKAIINYQYNQYTEEEKNNINFNIDDLSSKLNESINSAMININKFL |
| | | DQCSVSYLMNSMIPYAVKRLKDFDASVRDVLLKYIYDNRGTLVLQVDRLKD |
| | | EVNNTLSADIPFQLSKYVDNKKLLSTFTEYIKN |
| 87 | LC BoNT/A1-WT | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 88 | LC BoNT/A1-K11R | MPFVNKQFNYRDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 89 | LC BoNT/A1-K41R | MPFVNKQFNYKDPVNGVDIAYIKIPNAGQMQPVKAFKIHNRIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 90 | LC BoNT/A1-K212R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GRFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 91 | LC BoNT/A1-K272R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDARFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 92 | LC BoNT/A1-K289R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 93 | LC BoNT/A1-K291R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFRDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 94 | LC BoNT/A1-K299R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNRAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 95 | LC BoNT/A1-K318R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 96 | LC BoNT/A1-K335R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDRLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 97 | LC BoNT/A1-K337R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLRFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 98 | LC BoNT/A1-K340R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDRLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 99 | LC BoNT/A1-K343R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYRMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 100 | LC BoNT/A1-K356R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVRFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 101 | LC BoNT/A1-K375R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFRINIVPKVNYTIYDGFNLRNTNLAANFNGQNTEINNMNFTKLKN |
| | | FTGLFEFYKLLCVRGIITSKTKS |
| 102 | LC BoNT/A1-K381R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPRVNYTIYDGFNLRNTNLAANFNGQNTEINNMNFTKLKN |
| | | FTGLFEFYKLLCVRGIITSKTKS |
| 103 | LC BoNT/A1-Y387R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIRDGFNLRNTNLAANFNGQNTEINNMNFTKLKN |
| | | FTGLFEFYKLLCVRGIITSKTKS |
| 104 | LC BoNT/A1-M411R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNRNFTKLKN |
| | | FTGLFEFYKLLCVRGIITSKTKS |
| 105 | LC BoNT/A1-K415R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTRLKN |
| | | FTGLFEFYKLLCVRGIITSKTKS |
| 106 | LC BoNT/A1-Group 0 | MPFVNKQFNYRDPVNGVDIAYIKIPNAGQMQPVKAFKIHNKIWVIPERDT |
| | (K11R, K335R, K337R, | FTNPEEGDLNPPPEAKQVPVSYYDSTYLSTDNEKDNYLKGVTKLFERIYSTDL |
| | K343R, K375R, K415R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDRLRFDKLYRMLTEIYTEDNFVKFFKVLNRKTYL |
| | | NFDKAVFRINIVPKVNYTIYDGFNLRNTNLAANFNGQNTEINNMNFTRLKN |
| | | FTGLFEFYKLLCVRGIITSKTKS |
| 107 | LC BoNT/A1-Group 1 | MPFVNKQFNYKDPVNGVDIAYIKIPNAGQMQPVKAFKIHNRIWVIPERDT |
| | (K41R, K318R, K340R) | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDKLKFDRLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 108 | LC BoNT/A1-Group 2 | MPFVNKQFNYKDPVNGVDIAYIKIPNAGQMQPVKAFKIHNKIWVIPERDT |
| | (K289R, K291R, | FTNPEEGDLNPPPEAKQVPVSYYDSTYLSTDNEKDNYLKGVTKLFERIYSTDL |
| | K299R, K381R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDKLKFDKLYKMLTEIYTEDNFVKFFKVLNRKTYL |
| | | NFDKAVFKINIVPRVNYTIYDGFNLRNTNLAANFNGQNTEINNMNFTKLKN |
| | | FTGLFEFYKLLCVRGIITSKTKS |
| 109 | LC BoNT/A1-Group 3 | MPFVNKQFNYKDPVNGVDIAYIKIPNAGQMQPVKAFKIHNKIWVIPERDT |
| | (K212R, K272R, | FTNPEEGDLNPPPEAKQVPVSYYDSTYLSTDNEKDNYLKGVTKLFERIYSTDL |
| | K356R) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GRFATDPAVTLAHELIHAGHRLYGIAINPNRVFKVNTNAYYEMSGLEVSFEE |
| | | LRTFGGHDARFIDSLQENEFRLYYYNKFKDIASTLNKAKSIVGTTASLQYMK |
| | | NVFKEKYLLSEDTSGKFSVDKLKFDKLYKMLTEIYTEDNFVRFFKVLNRKTYL |
| | | NFDKAVFKINIVPKVNYTIYDGFNLRNTNLAANFNGQNTEINNMNFTKLKN |
| | | FTGLFEFYKLLCVRGIITSKTKS |
| 110 | LC BoNT/A1-KR9 | MPFVNKQFNYRDPVNGVDIAYIKIPNAGQMQPVKAFKIHNRIWVIPERDT |
| | (K11R, K41R, K318R, | FTNPEEGDLNPPPEAKQVPVSYYDSTYLSTDNEKDNYLKGVTKLFERIYSTDL |
| | K335R, K337R, K340R, | GRMLLTSIVRGIPFWGGSTIDTELKVIDTNCINVIQPDGSYRSEELNLVIIGPS |
| | K343R, K375R, K415R) | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFREKYLLSEDTSGKFSVDRLRFDRLYRMLTEIYTEDNFVKFFKVLNRKTYL |
| | | NFDKAVFRINIVPKVNYTIYDGFNLRNTNLAANFNGQNTEINNMNFTRLKN |
| | | FTGLFEFYKLLCVRGIITSKTKS |
| 111 | LC BoNT/A1-KR10 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K343R, K375R, K381R, | ADIIQFECKSFGHEVLNLTRNGYGSTQYIRFSPDFTFGFEESLEVDTNPLLGA |
| | K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 112 | LC BoNT/A1-KR11-41 | MPFVNKQFNYRDPVNGVDIAYIKIPNAGQMQPVKAFKIHNRIWVIPERDT |
| | (K11R, K41R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 113 | LC BoNT/A1-KR11-212 | MPFVNKQFNYRDPVNGVDIAYIKIPNAGQMQPVKAFKIHNKIWVIPERDT |
| | (K11R, K212R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GRFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 114 | LC BoNT/A1-KR11-272 | MPFVNKQFNYRDPVNGVDIAYIKIPNAGQMQPVKAFKIHNKIWVIPERDT |
| | (K11R, K272R, K289R, | FTNPEEGDLNPPPEAKQVPVSYYDSTYLSTDNEKDNYLKGVTKLFERIYSTDL |
| | K291R, K299R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDARFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 115 | LC BoNT/A1-KR11-318 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K318R, K335R, | GRMLLTSIVRGIPFWGGSTIDTELKVIDTNCINVIQPDGSYRSEELNLVIIGPS |
| | K337R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFREKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 116 | LC BoNT/A1-KR11-340 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K340R, K343R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDRLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 117 | LC BoNT/A1-KR11-356 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K343R, K356R, K375R, | ADIIQFECKSFGHEVLNLTRNGYGSTQYIRFSPDFTFGFEESLEVDTNPLLGA |
| | K381R, K415R) | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 118 | LC BoNT/A1-KR13 | MPFVNKQFNYRDPVNGVDIAYIKIPNAGQMQPVKAFKIHNRIWVIPERDT |
| | (K11R, K41R, K289R, | FTNPEEGDLNPPPEAKQVPVSYYDSTYLSTDNEKDNYLKGVTKLFERIYSTDL |
| | K291R, K299R, K318R, | GRMLLTSIVRGIPFWGGSTIDTELKVIDTNCINVIQPDGSYRSEELNLVIIGPS |
| | K335R, K337R, K340R, | ADIIQFECKSFGHEVLNLTRNGYGSTQYIRFSPDFTFGFEESLEVDTNPLLGA |
| | K343R, K375R, K381R, | GKFATDPAVTLAHELIHAGHRLYGIAINPNRVFKVNTNAYYEMSGLEVSFEE |
| | K415R) | LRTFGGHDAKFIDSLQENEFRLYYYNRFRDIASTLNRAKSIVGTTASLQYMK |
| | | NVFREKYLLSEDTSGKFSVDRLRFDRLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 119 | LC BoNT/A1-KR15 | MPFVNKQFNYRDPVNGVDIAYIKIPNAGQMQPVKAFKIHNRIWVIPERDT |
| | (K11R, K41R, K212R, | FTNPEEGDLNPPPEAKQVPVSYYDSTYLSTDNEKDNYLKGVTKLFERIYSTDL |
| | K289R, K291R, K299R, | GRMLLTSIVRGIPFWGGSTIDTELKVIDTNCINVIQPDGSYRSEELNLVIIGPS |
| | K318R, K335R, K337R, | ADIIQFECKSFGHEVLNLTRNGYGSTQYIRFSPDFTFGFEESLEVDTNPLLGA |
| | K340R, K343R, K356R, | GRFATDPAVTLAHELIHAGHRLYGIAINPNRVFKVNTNAYYEMSGLEVSFEE |
| | K375R, K381R, K415R) | LRTFGGHDAKFIDSLQENEFRLYYYNRFRDIASTLNRAKSIVGTTASLQYMK |
| | | NVFREKYLLSEDTSGKFSVDRLRFDRLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 120 | LC BoNT/A1-KR16 | MPFVNKQFNYRDPVNGVDIAYIKIPNAGQMQPVKAFKIHNRIWVIPERDT |
| | (K11R, K41R, K212R, | FTNPEEGDLNPPPEAKQVPVSYYDSTYLSTDNEKDNYLKGVTKLFERIYSTDL |
| | K272R, K289R, K291R, | GRMLLTSIVRGIPFWGGSTIDTELKVIDTNCINVIQPDGSYRSEELNLVIIGPS |
| | K299R, K318R, K335R, | ADIIQFECKSFGHEVLNLTRNGYGSTQYIRFSPDFTFGFEESLEVDTNPLLGA |
| | K337R, K340R, K343R, | GRFATDPAVTLAHELIHAGHRLYGIAINPNRVFKVNTNAYYEMSGLEVSFEE |
| | K356R, K375R, K381R, | LRTFGGHDARFIDSLQENEFRLYYYNRFRDIASTLNRAKSIVGTTASLQYMK |
| | K415R) | NVFREKYLLSEDTSGKFSVDRLRFDRLYRMLTEIYTEDNFVRFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKS |
| 121 | LC BoNT/A2-WT | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 122 | LC BoNT/A2-K11R | MPFVNKQFNYRDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 123 | LC BoNT/A2-K289R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNRFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 124 | LC BoNT/A2-K291R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFRDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 125 | LC BoNT/A2-K299R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNRAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 126 | LC BoNT/A2-K335R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDRLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 127 | LC BoNT/A2-K337R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLRFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 128 | LC BoNT/A2-K343R | MPFVNKQFNYKDPVNGVDIAYIKIPNAGQMQPVKAFKIHNKIWVIPERDT |
| | | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 129 | LC BoNT/A2-KR7 | MPFVNKQFNYRDPVNGVDIAYIKIPNAGQMQPVKAFKIHNKIWVIPERDT |
| | (K11R, K289R, K291R, | FTNPEEGDLNPPPEAKQVPVSYYDSTYLSTDNEKDNYLKGVTKLFERIYSTDL |
| | K299R, K335R, K337R, | GRMLLTSIVRGIPFWGGSTIDTELKVIDTNCINVIQPDGSYRSEELNLVIIGPS |
| | K343R) | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNRFRDVASTLNRAKSIIGTTASLQYMK |
| | | NVFKEKYLLSEDTSGKFSVDRLRFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFKTKS |
| 131 | H_{C}-BoNT/A1-F917R (of | IINTSILNLRYESNHLIDLSRYASKINIGSKVNFDPIDKNQIQLRNLESSKIEVILK |
| | full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 132 | H_{C}-BoNT/A1-F917K (of | IINTSILNLRYESNHLIDLSRYASKINIGSKVNFDPIDKNQIQLKNLESSKIEVILK |
| | full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 133 | H_{C}-BoNT/A1-F953H | IINTSILNLRYESNHLIDLSRYASKINIGSKVNFDPIDKNQIQLFNLESSKIEVILK |
| | (of full length) | NAIVYNSMYENFSTSFWIRIPKYHNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 134 | H_{C}-BoNT/A1-F953Y (of | IINTSILNLRYESNHLIDLSRYASKINIGSKVNFDPIDKNQIQLFNLESSKIEVILK |
| | full length) | NAIVYNSMYENFSTSFWIRIPKYYNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 135 | H_{C}-BoNT/A1-N954S | IINTSILNLRYESNHLIDLSRYASKINIGSKVNFDPIDKNQIQLFNLESSKIEVILK |
| | (of full length) | NAIVYNSMYENFSTSFWIRIPKYFSSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 136 | H_{C}-BoNT/A1-S955K (of | IINTSILNLRYESNHLIDLSRYASKINIGSKVNFDPIDKNQIQLFNLESSKIEVILK |
| | full length) | NAIVYNSMYENFSTSFWIRIPKYFNKISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 137 | H_{C}-BoNT/A1-S957N | IINTSILNLRYESNHLIDLSRYASKINIGSKVNFDPIDKNQIQLFNLESSKIEVILK |
| | (of full length) | NAIVYNSMYENFSTSFWIRIPKYFNSINLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 138 | H_{C}-BoNT/A1-M968I | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCIENNSGWKVSLNYGEI |
| | | IWTLQDTQEIKQRVVFKYSQMINISDYINRWIFVTITNNRLNNSKIYINGRLI |
| | | DQKPISNLGNIHASNNIMFKLDGCRDTHRYIWIKYFNLFDKELNEKEIKDLY |
| | | DNQSNSGILKDFWGDYLQYDKPYYMLNLYDPNKYVDVNNVGIRGYMYLK |
| | | GPRGSVMTTNIYLNSSLYRGTKFIIKKYASGNKDNIVRNNDRVYINVVVKNK |
| | | EYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 139 | H_{C}-BoNT/A1-N1025T | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLTNSKIYINGRL |
| | | IDQKPISNLGNIHASNNIMFKLDGCRDTHRYIWIKYFNLFDKELNEKEIKDLY |
| | | DNQSNSGILKDFWGDYLQYDKPYYMLNLYDPNKYVDVNNVGIRGYMYLK |
| | | GPRGSVMTTNIYLNSSLYRGTKFIIKKYASGNKDNIVRNNDRVYINVVVKNK |
| | | EYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 140 | H_{C}-BoNT/A1 - N1026K | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNKSKIYINGRL |
| | | IDQKPISNLGNIHASNNIMFKLDGCRDTHRYIWIKYFNLFDKELNEKEIKDLY |
| | | DNQSNSGILKDFWGDYLQYDKPYYMLNLYDPNKYVDVNNVGIRGYMYLK |
| | | GPRGSVMTTNIYLNSSLYRGTKFIIKKYASGNKDNIVRNNDRVYINVVVKNK |
| | | EYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 141 | H_{C}-BoNT/A1 - N1052K | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNKIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 142 | H_{C}-BoNT/A1 - D1062E | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRETHRYIWIKYFNLFDKELNEKEIKDLY |
| | | DNQSNSGILKDFWGDYLQYDKPYYMLNLYDPNKYVDVNNVGIRGYMYLK |
| | | GPRGSVMTTNIYLNSSLYRGTKFIIKKYASGNKDNIVRNNDRVYINVVVKNK |
| | | EYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 143 | H_{C}-BoNT/A1 - T1063P | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDPHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 144 | H_{C}-BoNT/A1 - H1064R | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTRRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 145 | H_{C}-BoNT/A1 - H1064Q | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTQRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 146 | H_{C}-BoNT/A1 - R1065N | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHNYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 147 | H_{C}-BoNT/A1 - Y1066R | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRRIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 148 | H_{C}-BoNT/A1 - Y1066K | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRKIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 149 | H_{C}-BoNT/A1 - T1145Y | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMYTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 150 | H_{C}-BoNT/A1 - R1156M | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYMGTKFIIKKYASGNKDNIVRNNDRVYINVVVK |
| | | NKEYRLATNASQAGVEKILSALEIPDVGNLSQVVVMKSKNDQGITNKCKM |
| | | NLQDNNGNDIGFIGFHQFNNIAKLVASNWYNRQIERSSRTLGCSWEFIPV |
| | | DDGWGERPL |
| 151 | H_{C}-BoNT/A1 - R1156I | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYIGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 152 | H_{C}-BoNT/A1 - T1232R | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGIRNKCKMN |
| | | LQDNNGNDIGFIGFHQFNNIAKLVASNWYNRQIERSSRTLGCSWEFIPVDD |
| | | GWGERPL |
| 153 | H_{C}-BoNT/A1 - T1232K | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGIKNKCKMN |
| | | LQDNNGNDIGFIGFHQFNNIAKLVASNWYNRQIERSSRTLGCSWEFIPVDD |
| | | GWGERPL |
| 154 | H_{C}-BoNT/A1 - E1272G | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIGRSSRTLGCSWEFIPVDD |
| | | GWGERPL |
| 155 | H_{C}-BoNT/A1-L1278F | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTFGCSWEFIPVDD |
| | | GWGERPL |
| 156 | H_{C}-BoNT/A1-L1278Y | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTYGCSWEFIPVDD |
| | | GWGERPL |
| 157 | H_{C}-BoNT/A1-L1278W | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTWGCSWEFIPVD |
| | | DGWGERPL |
| 158 | H_{C}-BoNT/A1-D1288E | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVED |
| | | GWGERPL |
| 159 | H_{C}-BoNT/A1-D1289Y | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDY |
| | | GWGERPL |
| 160 | H_{C}-BoNT/A1-G1292R | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWRERPL |
| 161 | H_{C}-BoNT/A1-G1292K | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWKERPL |
| 162 | H_{C}-BoNT/A1-R1294S | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGESPL |
| 163 | H_{C}-BoNT/A1-R1294T | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGETPL |
| 164 | H_{C}-BoNT/A1-P1295S | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERSL |
| 165 | H_{C}-BoNT/A1-P1295T | IINTSILNLRYESNHLIDLSRYASKINIGSKVNFDPIDKNQIQLFNLESSKIEVILK |
| | (of full length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERTL |
| 166 | H_{C}-BoNT/A2-K915Q | IVNTSILSIVYKKDDLIDLSRYGAKINIGDRVYYDSIDKNQIQLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 167 | H_{C}-BoNT/A2-T923K (of | IVNTSILSIVYKKDDLIDLSRYGAKINIGDRVYYDSIDKNQIKLINLESSKIEVILK |
| | full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 168 | H_{C}-BoNT/A2-S1090N | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | NQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 169 | H_{C}-BoNT/A2-N1103D | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGDYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 170 | H_{C}-BoNT/A2-F1117Y | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLYDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 171 | H_{C}-BoNT/A2-E1156M | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYMGTKFIIKKYASGNEDNIVRNNDRVYINVVVKNKEY |
| | | RLATNASQAGVEKILSALEIPDVGNLSQVVVMKSKDDQGIRNKCKMNLQD |
| | | NNGNDIGFIGFHLYDNIAKLVASNWYNRQVGKASRTFGCSWEFIPVDDG |
| | | WGESSL |
| 172 | H_{C}-BoNT/A2-E1170K | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNKDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 173 | H_{C}-BoNT/A2-D1227N | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKNDQGIRNKCKMNLQDN |
| | | NGNDIGFIGFHLYDNIAKLVASNWYNRQVGKASRTFGCSWEFIPVDDGW |
| | | GESSL |
| 174 | H_{C}-BoNT/A2-L1254Q | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHQYDNIAKLVASNWYNRQVGKASRTFGCSWEFIPVDDGW |
| | | GESSL |
| 175 | H_{C}-BoNT/A2-Y1255F | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLFDNIAKLVASNWYNRQVGKASRTFGCSWEFIPVDDGW |
| | | GESSL |
| 176 | H_{C}-BoNT/A2-D1256N | IVNTSILSIVYKKDDLIDLSRYGAKINIGDRVYYDSIDKNQIKLINLESSTIEVILK |
| | (of full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYNNIAKLVASNWYNRQVGKASRTFGCSWEFIPVDDGW |
| | | GESSL |
| 177 | BoNT/A1 - KR10/ | MPFVNKQFNYRDPVNGVDIAYIKIPNAGQMQPVKAFKIHNKIWVIPERDT |
| | R1156M (of full | FTNPEEGDLNPPPEAKQVPVSYYDSTYLSTDNEKDNYLKGVTKLFERIYSTDL |
| | length) | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKETL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYMGT |
| | | KFIIKKYASGNKDNIVRNNDRVYINVVVKNKEYRLATNASQAGVEKILSALEI |
| | | PDVGNLSQVVVMKSKNDQGITNKCKMNLQDNNGNDIGFIGFHQFNNIAK |
| | | LVASNWYNRQIERSSRTLGCSWEFIPVDDGWGERPL |
| 178 | BoNT/A1 - KR10/ | MPFVNKQFNYRDPVNGVDIAYIKIPNAGQMQPVKAFKIHNKIWVIPERDT |
| | T1232R (of full length) | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKETL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNIINTSILNLRYESNHLIDLSRYASK |
| | | INIGSKVNFDPIDKNQIQLFNLESSKIEVILKNAIVYNSMYENFSTSFWIRIPKY |
| | | FNSISLNNEYTIINCMENNSGWKVSLNYGEIIWTLQDTQEIKQRVVFKYSQ |
| | | MINISDYINRWIFVTITNNRLNNSKIYINGRLIDQKPISNLGNIHASNNIMFKL |
| | | DGCRDTHRYIWIKYFNLFDKELNEKEIKDLYDNQSNSGILKDFWGDYLQYD |
| | | KPYYMLNLYDPNKYVDVNNVGIRGYMYLKGPRGSVMTTNIYLNSSLYRGT |
| | | KFIIKKYASGNKDNIVRNNDRVYINVVVKNKEYRLATNASQAGVEKILSALEI |
| | | PDVGNLSQVVVMKSKNDQGIRNKCKMNLQDNNGNDIGFIGFHQFNNIA |
| | | KLVASNWYNRQIERSSRTLGCSWEFIPVDDGWGERPL |
| 179 | H_{C}-BoNT/B | SEILNNIILNLRYRDNNLIDLSGYGAKVEVYDGVKLNDKNQFKLTSSADSKIR |
| | | VTQNQNIIFNSMFLDFSVSFWIRIPKYRNDDIQNYIHNEYTIINCMKNNSG |
| | | WKISIRGNRIIWTLIDINGKTKSVFFEYNIREDISEYINRWFFVTITNNLDNAKI |
| | | YINGTLESNMDIKDIGEVIVNGEITFKLDGDVDRTQFIWMKYFSIFNTQLNQ |
| | | SNIKEIYKIQSYSEYLKDFWGNPLMYNKEYYMFNAGNKNSYIKLVKDSSVGE |
| | | ILIRSKYNQNSNYINYRNLYIGEKFIIRRESNSQSINDDIVRKEDYIHLDLVLHH |
| | | EEWRVYAYKYFKEQEEKLFLSIISDSNEFYKTIEIKEYDEQPSYSCQLLFKKDEE |
| | | STDDIGLIGIHRFYESGVLRKKYKDYFCISKWYLKEVKRKPYKSNLGCNWQFI |
| | | PKDEGWTE |
| 180 | H_{C}-BoNT/C | SKILSLQNRKNTLVDTSGYNAEVSEEGDVQLNPIFPFDFKLGSSGEDRGKVIV |
| | | TQNENIVYNSMYESFSISFWIRINKWVSNLPGYTIIDSVKNNSGWSIGIISNF |
| | | LVFTLKQNEDSEQSINFSYDISNNAPGYNKWFFVTVTNNMMGNMKIYING |
| | | KLIDTIKVKELTGINFSKTITFEINKIPDTGLITSDSDNINMWIRDFYIFAKELDG |
| | | KDINILFNSLQYTNVVKDYWGNDLRYNKEYYMVNIDYLNRYMYANSRQIV |
| | | FNTRRNNNDFNEGYKIIIKRIRGNTNDTRVRGGDILYFDMTINNKAYNLFM |
| | | KNETMYADNHSTEDIYAIGLREQTKDINDNIIFQIQPMNNTYYYASQIFKSN |
| | | FNGENISGICSIGTYRFRLGGDWYRHNYLVPTVKQGNYASLLESTSTHWGF |
| | | VPVSE |
| 181 | H_{C}-BoNT/D | SINDSKILSLQNKKNALVDTSGYNAEVRVGDNVQLNTIYTNDFKLSSSGDKII |
| | | VNLNNNILYSAIYENSSVSFWIKISKDLTNSHNEYTIINSIEQNSGWKLCIRNG |
| | | NIEWILQDVNRKYKSLIFDYSESLSHTGYTNKWFFVTITNNIMGYMKLYING |
| | | ELKQSQKIEDLDEVKLDKTIVFGIDENIDENQMLWIRDFNIFSKELSNEDINIV |
| | | YEGQILRNVIKDYWGNPLKFDTEYYIINDNYIDRYIAPESNVLVLVQYPDRSK |
| | | LYTGNPITIKSVSDKNPYSRILNGDNIILHMLYNSRKYMIIRDTDTIYATQGGE |
| | | CSQNCVYALKLQSNLGNYGIGIFSIKNIVSKNKYCSQIFSSFRENTMLLADIYK |
| | | PWRFSFKNAYTPVAVTNYETKLLSTSSFWKFISRDPGWVE |
| 182 | H_{C}-BoNT/E | KRIKSSSVLNMRYKNDKYVDTSGYDSNININGDVYKYPTNKNQFGIYNDKLS |
| | | EVNISQNDYIIYDNKYKNFSISFWVRIPNYDNKIVNVNNEYTIINCMRDNNS |
| | | GWKVSLNHNEIIWTLQDNAGINQKLAFNYGNANGISDYINKWIFVTITNDR |
| | | LGDSKLYINGNLIDQKSILNLGNIHVSDNILFKIVNCSYTRYIGIRYFNIFDKEL |
| | | DETEIQTLYSNEPNTNILKDFWGNYLLYDKEYYLLNVLKPNNFIDRRKDSTLSI |
| | | NNIRSTILLANRLYSGIKVKIQRVNNSSTNDNLVRKNDQVYINFVASKTHLFP |
| | | LYADTATTNKEKTIKISSSGNRFNQVVVMNSVGNNCTMNFKNNNGNNIG |
| | | LLGFKADTVVASTWYYTHMRDHTNSNGCFWNFISEEHGWQEK |
| 183 | H_{C}-BoNT/F | KKIKDNSILDMRYENNKFIDISGYGSNISINGDVYIYSTNRNQFGIYSSKPSEV |
| | | NIAQNNDIIYNGRYQNFSISFWVRIPKYFNKVNLNNEYTIIDCIRNNNSGWK |
| | | ISLNYNKIIWTLQDTAGNNQKLVFNYTQMISISDYINKWIFVTITNNRLGNS |
| | | RIYINGNLIDEKSISNLGDIHVSDNILFKIVGCNDTRYVGIRYFKVFDTELGKTE |
| | | IETLYSDEPDPSILKDFWGNYLLYNKRYYLLNLLRTDKSITQNSNFLNINQQR |
| | | GVYQKPNIFSNTRLYTGVEVIIRKNGSTDISNTDNFVRKNDLAYINVVDRDV |
| | | EYRLYADISIAKPEKIIKLIRTSNSNNSLGQIIVMDSIGNNCTMNFQNNNGG |
| | | NIGLLGFHSNNLVASSWYYNNIRKNTSSNGCFWSFISKEHGWQEN |
| 184 | H_{C}-BoNT/G | SNISSNAILSLSYRGGRLIDSSGYGATMNVGSDVIFNDIGNGQFKLNNSENS |
| | | NITAHQSKFVVYDSMFDNFSINFWVRTPKYNNNDIQTYLQNEYTIISCIKND |
| | | SGWKVSIKGNRIIWTLIDVNAKSKSIFFEYSIKDNISDYINKWFSITITNDRLG |
| | | NANIYINGSLKKSEKILNLDRINSSNDIDFKLINCTDTTKFVWIKDFNIFGREL |
| | | NATEVSSLYWIQSSTNTLKDFWGNPLRYDTQYYLFNQGMQNIYIKYFSKAS |
| | | MGETAPRTNFNNAAINYQNLYLGLRFIIKKASNSRNINNDNIVREGDYIYLNI |
| | | DNISDESYRVYVLVNSKEIQTQLFLAPINDDPTFYDVLQIKKYYEKTTYNCQIL |
| | | CEKDTKTFGLFGIGKFVKDYGYVWDTYDNYFCISQWYLRRISENINKLRLGC |
| | | NWQFIPVDEGWTE |
| 185 | H_{C}-BoNT/H | GELKYNCILNIKYEMDRDKLVDSSGYRSRINIGTGVKFSEIDKNQVQLSNLE |
| | | SSKIEVILNNGVIYNSMYENFSTSFWIRIPKYFRNINNEYKIISCMQNNSGWE |
| | | VSLNFSNMNSKIIWTLQDTEGIKKTVVFQYTQNINISDYINRWIFVTITNNRL |
| | | SNSKIYINGRLINEESISDLGNIHASNNIMFKLDGCRDPHRYIWIKYFNLFDKE |
| | | LNKKEIKDLYDNQSNSGILKDFWGDYLQYDKPYYMLNLYDPNKYLDVNNV |
| | | GIRGYMYLKGPRGRIVTTNIYLNSTLYMGTKFIIKKYASGNKDNIVRNNDRV |
| | | YINVVVKNKEYRLATNASQAGVEKILSAVEIPDVGNLSQVVVMKSENDQGI |
| | | RNKCKMNLQDNNGNDIGFIGFHQFNNIAKLVASNWYNRQIGKASRTFGC |
| | | SWEFIPVDDGWGESSL |
| 186 | H_{C}-BoNT/EN | GKIQDLSGENTPLTLGENLHIVNGRDNQAVRLNNQLDSKLEIQSRPNIHFTA |
| | | FEDFSISIWIRCSMLRNNRNRGQKYTIIQQFNKYGWQLAIQDSVFVWTLHD |
| | | TFNNQIQLTSGSALTNKNYLLQNFWLHITVTNKRSEKSRLYINGVLQDQKDI |
| | | SVLGNCHPKEPILFSIQDNSDPNYFVRFEQFNVYRKALTDSEVNRLYWKYFE |
| | | GSYLRDVWGERLTYNRDYYMQLSTLPGRGIKREYRTWSGFDYIILSELGTQK |
| | | IPTHEVTYPKLYQGQKITIHSDGKNLEPHVKSNKNIRLKIDDFYIGVVNPFKLP |
| | | EWRPESGAYVVTTYNHAEDLCLYFRTRSSSQSLYYGQLIMNDGRNKSLLNY |
| | | TLKGSTYWIWSSAWYYENYNTSSKTAGNWYFIPVDEGWKED |
| 187 | LC-Hn BoNT/A1 - H_{C}- | SLDKGYNKALNDLCIKVNNWDLFFSPSEDNFTNDLNKGEEITSDTNIEAAEE |
| | BoNT/B | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLSEILNNIILNLRYRDNNLIDLSGYGAKVEVYDGVKLND |
| | | KNQFKLTSSADSKIRVTQNQNIIFNSMFLDFSVSFWIRIPKYRNDDIQNYIHN |
| | | EYTIINCMKNNSGWKISIRGNRIIWTLIDINGKTKSVFFEYNIREDISEYINRW |
| | | FFVTITNNLDNAKIYINGTLESNMDIKDIGEVIVNGEITFKLDGDVDRTQFIW |
| | | MKYFSIFNTQLNQSNIKEIYKIQSYSEYLKDFWGNPLMYNKEYYMFNAGNK |
| | | NSYIKLVKDSSVGEILIRSKYNQNSNYINYRNLYIGEKFIIRRESNSQSINDDIV |
| | | RKEDYIHLDLVLHHEEWRVYAYKYFKEQEEKLFLSIISDSNEFYKTIEIKEYDEQ |
| | | PSYSCQLLFKKDEESTDDIGLIGIHRFYESGVLRKKYKDYFCISKWYLKEVKRK |
| | | PYKSNLGCNWQFIPKDEGWTE |
| 188 | LC-Hn BoNT/A1 - H_{C} | SLDKGYNKALNDLCIKVNNWDLFFSPSEDNFTNDLNKGEEITSDTNIEAAEE |
| | BoNT/C | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLSKILSLQNRKNTLVDTSGYNAEVSEEGDVQLNPIFPFD |
| | | FKLGSSGEDRGKVIVTQNENIVYNSMYESFSISFWIRINKWVSNLPGYTIIDS |
| | | VKNNSGWSIGIISNFLVFTLKQNEDSEQSINFSYDISNNAPGYNKWFFVTVT |
| | | NNMMGNMKIYINGKLIDTIKVKELTGINFSKTITFEINKIPDTGLITSDSDNIN |
| | | MWIRDFYIFAKELDGKDINILFNSLQYTNVVKDYWGNDLRYNKEYYMVNI |
| | | DYLNRYMYANSRQIVFNTRRNNNDFNEGYKIIIKRIRGNTNDTRVRGGDILY |
| | | FDMTINNKAYNLFMKNETMYADNHSTEDIYAIGLREQTKDINDNIIFQIQP |
| | | MNNTYYYASQIFKSNFNGENISGICSIGTYRFRLGGDWYRHNYLVPTVKQG |
| | | NYASLLESTSTHWGFVPVSE |
| 189 | LC-Hn BoNT/A1 - H_{C}- | SLDKGYNKALNDLCIKVNNWDLFFSPSEDNFTNDLNKGEEITSDTNIEAAEE |
| | BoNT/D | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLSINDSKILSLQNKKNALVDTSGYNAEVRVGDNVQLNT |
| | | IYTNDFKLSSSGDKIIVNLNNNILYSAIYENSSVSFWIKISKDLTNSHNEYTIINS |
| | | IEQNSGWKLCIRNGNIEWILQDVNRKYKSLIFDYSESLSHTGYTNKWFFVTIT |
| | | NNIMGYMKLYINGELKQSQKIEDLDEVKLDKTIVFGIDENIDENQMLWIRD |
| | | FNIFSKELSNEDINIVYEGQILRNVIKDYWGNPLKFDTEYYIINDNYIDRYIAPE |
| | | SNVLVLVQYPDRSKLYTGNPITIKSVSDKNPYSRILNGDNIILHMLYNSRKYM |
| | | IIRDTDTIYATQGGECSQNCVYALKLQSNLGNYGIGIFSIKNIVSKNKYCSQIF |
| | | SSFRENTMLLADIYKPWRFSFKNAYTPVAVTNYETKLLSTSSFWKFISRDPG |
| | | WVE |
| 190 | LC-Hn BoNT/A1 - H_{C}- | SLDKGYNKALNDLCIKVNNWDLFFSPSEDNFTNDLNKGEEITSDTNIEAAEE |
| | BoNT/E | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLKRIKSSSVLNMRYKNDKYVDTSGYDSNININGDVYKY |
| | | PTNKNQFGIYNDKLSEVNISQNDYIIYDNKYKNFSISFWVRIPNYDNKIVNV |
| | | NNEYTIINCMRDNNSGWKVSLNHNEIIWTLQDNAGINQKLAFNYGNANG |
| | | ISDYINKWIFVTITNDRLGDSKLYINGNLIDQKSILNLGNIHVSDNILFKIVNCS |
| | | YTRYIGIRYFNIFDKELDETEIQTLYSNEPNTNILKDFWGNYLLYDKEYYLLNV |
| | | LKPNNFIDRRKDSTLSINNIRSTILLANRLYSGIKVKIQRVNNSSTNDNLVRKN |
| | | DQVYINFVASKTHLFPLYADTATTNKEKTIKISSSGNRFNQVVVMNSVGNN |
| | | CTMNFKNNNGNNIGLLGFKADTVVASTWYYTHMRDHTNSNGCFWNFIS |
| | | EEHGWQEK |
| 191 | LC-Hn BoNT/A1 - H_{C}- | SLDKGYNKALNDLCIKVNNWDLFFSPSEDNFTNDLNKGEEITSDTNIEAAEE |
| | BoNT/F | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLKKIKDNSILDMRYENNKFIDISGYGSNISINGDVYIYST |
| | | NRNQFGIYSSKPSEVNIAQNNDIIYNGRYQNFSISFWVRIPKYFNKVNLNNE |
| | | YTIIDCIRNNNSGWKISLNYNKIIWTLQDTAGNNQKLVFNYTQMISISDYIN |
| | | KWIFVTITNNRLGNSRIYINGNLIDEKSISNLGDIHVSDNILFKIVGCNDTRYV |
| | | GIRYFKVFDTELGKTEIETLYSDEPDPSILKDFWGNYLLYNKRYYLLNLLRTDK |
| | | SITQNSNFLNINQQRGVYQKPNIFSNTRLYTGVEVIIRKNGSTDISNTDNFVR |
| | | KNDLAYINVVDRDVEYRLYADISIAKPEKIIKLIRTSNSNNSLGQIIVMDSIGN |
| | | NCTMNFQNNNGGNIGLLGFHSNNLVASSWYYNNIRKNTSSNGCFWSFIS |
| | | KEHGWQEN |
| 192 | LC-Hn BoNT/A1 - H_{C}- | SLDKGYNKALNDLCIKVNNWDLFFSPSEDNFTNDLNKGEEITSDTNIEAAEE |
| | BoNT/G | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLSNISSNAILSLSYRGGRLIDSSGYGATMNVGSDVIFNDI |
| | | GNGQFKLNNSENSNITAHQSKFVVYDSMFDNFSINFWVRTPKYNNNDIQT |
| | | YLQNEYTIISCIKNDSGWKVSIKGNRIIWTLIDVNAKSKSIFFEYSIKDNISDYI |
| | | NKWFSITITNDRLG |
| | | NANIYINGSLKKSEKILNLDRINSSNDIDFKLINCTDTTKFVWIKDFNIFGREL |
| | | NATEVSSLYWIQSSTNTLKDFWGNPLRYDTQYYLFNQGMQNIYIKYFSKAS |
| | | MGETAPRTNFNNAAINYQNLYLGLRFIIKKASNSRNINNDNIVREGDYIYLNI |
| | | DNISDESYRVYVLVNSKEIQTQLFLAPINDDPTFYDVLQIKKYYEKTTYNCQIL |
| | | CEKDTKTFGLFGIGKFVKDYGYVWDTYDNYFCISQWYLRRISENINKLRLGC |
| | | NWQFIPVDEGWTE |
| 193 | LC-Hn BoNT/A1 - H_{C}- | SLDKGYNKALNDLCIKVNNWDLFFSPSEDNFTNDLNKGEEITSDTNIEAAEE |
| | BoNT/H | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLGELKYNCILNIKYEMDRDKLVDSSGYRSRINIGTGVKF |
| | | SEIDKNQVQLSNLESSKIEVILNNGVIYNSMYENFSTSFWIRIPKYFRNINNEY |
| | | KIISCMQNNSGWEVSLNFSNMNSKIIWTLQDTEGIKKTVVFQYTQNINISD |
| | | YINRWIFVTITNNRLSNSKIYINGRLINEESISDLGNIHASNNIMFKLDGCRDP |
| | | HRYIWIKYFNLFDKELNKKEIKDLYDNQSNSGILKDFWGDYLQYDKPYYML |
| | | NLYDPNKYLDVNNVGIRGYMYLKGPRGRIVTTNIYLNSTLYMGTKFIIKKYA |
| | | SGNKDNIVRNNDRVYINVVVKNKEYRLATNASQAGVEKILSAVEIPDVGNL |
| | | SQVVVMKSENDQGIRNKCKMNLQDNNGNDIGFIGFHQFNNIAKLVASN |
| | | WYNRQIGKASRTFGCSWEFIPVDDGWGESSL |
| 194 | LC-Hn BoNT/A1 - H_{C}- | SLDKGYNKALNDLCIKVNNWDLFFSPSEDNFTNDLNKGEEITSDTNIEAAEE |
| | BoNT/EN | NISLDLIQQYYLTFNFDNEPENISIENLSSDIIGQLELMPNIERFPNGKKYELDK |
| | | YTMFHYLRAQEFEHGKSRIALTNSVNEALLNPSRVYTFFSSDYVKKVNKATE |
| | | AAMFLGWVEQLVYDFTDETSEVSTTDKIADITIIIPYIGPALNIGNMLYKDDF |
| | | VGALIFSGAVILLEFIPEIAIPVLGTFALVSYIANKVLTVQTIDNALSKRNEKWD |
| | | EVYKYIVTNWLAKVNTQIDLIRKKMKETLENQAEATKAIINYQYNQYTEEEK |
| | | NNINFNIDDLSSKLNESINKAMININKFLNQCSVSYLMNSMIPYGVKRLEDF |
| | | DASLKDALLKYIYDNRGTLIGQVDRLKDKVNNTLSTDIPFQLSKYVDNQRLLS |
| | | TFTEYIKNIINTSILNLRYESNHLIDLSRYASKINIGSKVNFDPIDKNQIQLFNLE |
| | | SSKIEVILKNAIVYNSMYENFSTSFWIRIPKYFNSISLNNEYTIINCMENNSGW |
| | | KVSLNYGEIIWTLQDTQEIKQRVVFKYSQMINISDYINRWIFVTITNNRLNNS |
| | | KIYINGRLIDQKPISNLGNIHASNNIMFKLDGCRDTHRYIWIKYFNLFDKELN |
| | | EKEIKDLYDNQSNSGILKDFWGDYLQYDKPYYMLNLYDPNKYVDVNNVGI |
| | | RGYMYLKGPRGSVMTTNIYLNSSLYRGTKFIIKKYASGNKDNIVRNNDRVYI |
| | | NVVVKNKEYRLATNASQAGVEKILSALEIPDVGNLSQVVVMKSKNDQGITN |
| | | KCKMNLQDNNGNDIGFIGFHQFNNIAKLVASNWYNRQIERSSRTLGCSW |
| | | EFIPVDDGWGERPLGKIQDLSGENTPLTLGENLHIVNGRDNQAVRLNNQL |
| | | DSKLEIQSRPNIHFTAFEDFSISIWIRCSMLRNNRNRGQKYTIIQQFNKYGW |
| | | QLAIQDSVFVWTLHDTFNNQIQLTSGSALTNKNYLLQNFWLHITVTNKRSE |
| | | KSRLYINGVLQDQKDISVLGNCHPKEPILFSIQDNSDPNYFVRFEQFNVYRK |
| | | ALTDSEVNRLYWKYFEGSYLRDVWGERLTYNRDYYMQLSTLPGRGIKREYR |
| | | TWSGFDYIILSELGTQKIPTHEVTYPKLYQGQKITIHSDGKNLEPHVKSNKNI |
| | | RLKIDDFYIGVVNPFKLPEWRPESGAYVVTTYNHAEDLCLYFRTRSSSQSLYY |
| | | GQLIMNDGRNKSLLNYTLKGSTYWIWSSAWYYENYNTSSKTAGNWYFIPV |
| | | DEGWKED |
| 195 | LC-Hn BoNT/A2 - H_{C}- | IVNTSILSIVYKKDDLIDLSRYGAKINIGDRVYYDSIDKNQIKLINLESSTIEVILK |
| | BoNT/B | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNSEILNNIILNLRYRDNNLIDLSGYGAKVEVYDGVKL |
| | | NDKNQFKLTSSADSKIRVTQNQNIIFNSMFLDFSVSFWIRIPKYRNDDIQNYI |
| | | HNEYTIINCMKNNSGWKISIRGNRIIWTLIDINGKTKSVFFEYNIREDISEYIN |
| | | RWFFVTITNNLDNAKIYINGTLESNMDIKDIGEVIVNGEITFKLDGDVDRTQ |
| | | FIWMKYFSIFNTQLNQSNIKEIYKIQSYSEYLKDFWGNPLMYNKEYYMFNA |
| | | GNKNSYIKLVKDSSVGEILIRSKYNQNSNYINYRNLYIGEKFIIRRESNSQSIND |
| | | DIVRKEDYIHLDLVLHHEEWRVYAYKYFKEQEEKLFLSIISDSNEFYKTIEIKEY |
| | | DEQPSYSCQLLFKKDEESTDDIGLIGIHRFYESGVLRKKYKDYFCISKWYLKEV |
| | | KRKPYKSNLGCNWQFIPKDEGWTE |
| 196 | LC-Hn BoNT/A2 - H_{C} | IVNTSILSIVYKKDDLIDLSRYGAKINIGDRVYYDSIDKNQIKLINLESSTIEVILK |
| | BoNT/C | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNSKILSLQNRKNTLVDTSGYNAEVSEEGDVQLNPIF |
| | | PFDFKLGSSGEDRGKVIVTQNENIVYNSMYESFSISFWIRINKWVSNLPGYTI |
| | | IDSVKNNSGWSIGIISNFLVFTLKQNEDSEQSINFSYDISNNAPGYNKWFFV |
| | | TVTNNMMGNMKIYINGKLIDTIKVKELTGINFSKTITFEINKIPDTGLITSDSD |
| | | NINMWIRDFYIFAKELDGKDINILFNSLQYTNVVKDYWGNDLRYNKEYYM |
| | | VNIDYLNRYMYANSRQIVFNTRRNNNDFNEGYKIIIKRIRGNTNDTRVRGG |
| | | DILYFDMTINNKAYNLFMKNETMYADNHSTEDIYAIGLREQTKDINDNIIFQ |
| | | IQPMNNTYYYASQIFKSNFNGENISGICSIGTYRFRLGGDWYRHNYLVPTVK |
| | | QGNYASLLESTSTHWGFVPVSE |
| 197 | LC-Hn BoNT/A2 - H_{C}- | IVNTSILSIVYKKDDLIDLSRYGAKINIGDRVYYDSIDKNQIKLINLESSTIEVILK |
| | BoNT/D | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNSINDSKILSLQNKKNALVDTSGYNAEVRVGDNVQ |
| | | LNTIYTNDFKLSSSGDKIIVNLNNNILYSAIYENSSVSFWIKISKDLTNSHNEYT |
| | | IINSIEQNSGWKLCIRNGNIEWILQDVNRKYKSLIFDYSESLSHTGYTNKWFF |
| | | VTITNNIMGYMKLYINGELKQSQKIEDLDEVKLDKTIVFGIDENIDENQML |
| | | WIRDFNIFSKELSNEDINIVYEGQILRNVIKDYWGNPLKFDTEYYIINDNYIDR |
| | | YIAPESNVLVLVQYPDRSKLYTGNPITIKSVSDKNPYSRILNGDNIILHMLYNS |
| | | RKYMIIRDTDTIYATQGGECSQNCVYALKLQSNLGNYGIGIFSIKNIVSKNKY |
| | | CSQIFSSFRENTMLLADIYKPWRFSFKNAYTPVAVTNYETKLLSTSSFWKFIS |
| | | RDPGWVE |
| 198 | LC-Hn BoNT/A2 - H_{C}- | IVNTSILSIVYKKDDLIDLSRYGAKINIGDRVYYDSIDKNQIKLINLESSTIEVILK |
| | BoNT/E | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNKRIKSSSVLNMRYKNDKYVDTSGYDSNININGDVY |
| | | KYPTNKNQFGIYNDKLSEVNISQNDYIIYDNKYKNFSISFWVRIPNYDNKIVN |
| | | VNNEYTIINCMRDNNSGWKVSLNHNEIIWTLQDNAGINQKLAFNYGNAN |
| | | GISDYINKWIFVTITNDRLGDSKLYINGNLIDQKSILNLGNIHVSDNILFKIVNC |
| | | SYTRYIGIRYFNIFDKELDETEIQTLYSNEPNTNILKDFWGNYLLYDKEYYLLN |
| | | VLKPNNFIDRRKDSTLSINNIRSTILLANRLYSGIKVKIQRVNNSSTNDNLVRK |
| | | NDQVYINFVASKTHLFPLYADTATTNKEKTIKISSSGNRFNQVVVMNSVGN |
| | | NCTMNFKNNNGNNIGLLGFKADTVVASTWYYTHMRDHTNSNGCFWNFI |
| | | SEEHGWQEK |
| 199 | LC-Hn BoNT/A2 - H_{C}- | IVNTSILSIVYKKDDLIDLSRYGAKINIGDRVYYDSIDKNQIKLINLESSTIEVILK |
| | BoNT/F | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNKKIKDNSILDMRYENNKFIDISGYGSNISINGDVYIY |
| | | STNRNQFGIYSSKPSEVNIAQNNDIIYNGRYQNFSISFWVRIPKYFNKVNLN |
| | | NEYTIIDCIRNNNSGWKISLNYNKIIWTLQDTAGNNQKLVFNYTQMISISDYI |
| | | NKWIFVTITNNRLGNSRIYINGNLIDEKSISNLGDIHVSDNILFKIVGCNDTRY |
| | | VGIRYFKVFDTELGKTEIETLYSDEPDPSILKDFWGNYLLYNKRYYLLNLLRTD |
| | | KSITQNSNFLNINQQRGVYQKPNIFSNTRLYTGVEVIIRKNGSTDISNTDNFV |
| | | RKNDLAYINVVDRDVEYRLYADISIAKPEKIIKLIRTSNSNNSLGQIIVMDSIG |
| | | NNCTMNFQNNNGGNIGLLGFHSNNLVASSWYYNNIRKNTSSNGCFWSFI |
| | | SKEHGWQEN |
| 200 | LC-Hn BoNT/A2 - H_{C}- | IVNTSILSIVYKKDDLIDLSRYGAKINIGDRVYYDSIDKNQIKLINLESSTIEVILK |
| | BoNT/G | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNSNISSNAILSLSYRGGRLIDSSGYGATMNVGSDVIF |
| | | NDIGNGQFKLNNSENSNITAHQSKFVVYDSMFDNFSINFWVRTPKYNNN |
| | | DIQTYLQNEYTIISCIKNDSGWKVSIKGNRIIWTLIDVNAKSKSIFFEYSIKDNI |
| | | SDYINKWFSITITNDRLG |
| | | NANIYINGSLKKSEKILNLDRINSSNDIDFKLINCTDTTKFVWIKDFNIFGREL |
| | | NATEVSSLYWIQSSTNTLKDFWGNPLRYDTQYYLFNQGMQNIYIKYFSKAS |
| | | MGETAPRTNFNNAAINYQNLYLGLRFIIKKASNSRNINNDNIVREGDYIYLNI |
| | | DNISDESYRVYVLVNSKEIQTQLFLAPINDDPTFYDVLQIKKYYEKTTYNCQIL |
| | | CEKDTKTFGLFGIGKFVKDYGYVWDTYDNYFCISQWYLRRISENINKLRLGC |
| | | NWQFIPVDEGWTE |
| 201 | LC-Hn BoNT/A2 - H_{C}- | IVNTSILSIVYKKDDLIDLSRYGAKINIGDRVYYDSIDKNQIKLINLESSTIEVILK |
| | BoNT/H | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNGELKYNCILNIKYEMDRDKLVDSSGYRSRINIGTGV |
| | | KFSEIDKNQVQLSNLE |
| | | SSKIEVILNNGVIYNSMYENFSTSFWIRIPKYFRNINNEYKIISCMQNNSGWE |
| | | VSLNFSNMNSKIIWTLQDTEGIKKTVVFQYTQNINISDYINRWIFVTITNNRL |
| | | SNSKIYINGRLINEESISDLGNIHASNNIMFKLDGCRDPHRYIWIKYFNLFDKE |
| | | LNKKEIKDLYDNQSNSGILKDFWGDYLQYDKPYYMLNLYDPNKYLDVNNV |
| | | GIRGYMYLKGPRGRIVTTNIYLNSTLYMGTKFIIKKYASGNKDNIVRNNDRV |
| | | YINVVVKNKEYRLATNASQAGVEKILSAVEIPDVGNLSQVVVMKSENDQGI |
| | | RNKCKMNLQDNNGNDIGFIGFHQFNNIAKLVASNWYNRQIGKASRTFGC |
| | | SWEFIPVDDGWGESSL |
| 202 | LC-Hn BoNT/A2 - H_{C}- | IVNTSILSIVYKKDDLIDLSRYGAKINIGDRVYYDSIDKNQIKLINLESSTIEVILK |
| | BoNT/EN | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSLSLDEGYNKALNDLCIKVNNWDLFFSPSEDNFTNDLDKVEEITADTNI |
| | | EAAEENISLDLIQQYYLTFDFDNEPENISIENLSSDIIGQLEPMPNIERFPNGK |
| | | KYELDKYTMFHYLRAQEFEHGDSRIILTNSAEEALLKPNVAYTFFSSKYVKKI |
| | | NKAVEAFMFLNWAEELVYDFTDETNEVTTMDKIADITIIVPYIGPALNIGN |
| | | MLSKGEFVEAIIFTGVVAMLEFIPEYALPVFGTFAIVSYIANKVLTVQTINNAL |
| | | SKRNEKWDEVYKYTVTNWLAKVNTQIDLIREKMKKALENQAEATKAIINYQ |
| | | YNQYTEEEKNNINFNIDDLSSKLNESINSAMININKFLDQCSVSYLMNSMIP |
| | | YAVKRLKDFDASVRDVLLKYIYDNRGTLVLQVDRLKDEVNNTLSADIPFQLS |
| | | KYVDNKKLLSTFTEYIKNGKIQDLSGENTPLTLGENLHIVNGRDNQAVRLNN |
| | | QLDSKLEIQSRPNIHFTAFEDFSISIWIRCSMLRNNRNRGQKYTIIQQFNKYG |
| | | WQLAIQDSVFVWTLHDTFNNQIQLTSGSALTNKNYLLQNFWLHITVTNKR |
| | | SEKSRLYINGVLQDQKDISVLGNCHPKEPILFSIQDNSDPNYFVRFEQFNVYR |
| | | KALTDSEVNRLYWKYFEGSYLRDVWGERLTYNRDYYMQLSTLPGRGIKREY |
| | | RTWSGFDYIILSELGTQKIPTHEVTYPKLYQGQKITIHSDGKNLEPHVKSNKN |
| | | IRLKIDDFYIGVVNPFKLPEWRPESGAYVVTTYNHAEDLCLYFRTRSSSQSLY |
| | | YGQLIMNDGRNKSLLNYTLKGSTYWIWSSAWYYENYNTSSKTAGNWYFIP |
| | | VDEGWKED |
| 203 | LC-H_{N} BoNT/A1 KR10 - | MPFVNKQFNYRDPVNGVDIAYIKIPNAGQMQPVKAFKIHNKIWVIPERDT |
| | H_{C}/B | FTNPEEGDLNPPPEAKQVPVSYYDSTYLSTDNEKDNYLKGVTKLFERIYSTDL |
| | | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSKTKSLDKGYNKALNDLCIKVNNWDLFFSPSEDN |
| | | FTNDLNKGEEITSDTNIEAAEENISLDLIQQYYLTFNFDNEPENISIENLSSDII |
| | | GQLELMPNIERFPNGKKYELDKYTMFHYLRAQEFEHGKSRIALTNSVNEALL |
| | | NPSRVYTFFSSDYVKKVNKATEAAMFLGWVEQLVYDFTDETSEVSTTDKIA |
| | | DITIIIPYIGPALNIGNMLYKDDFVGALIFSGAVILLEFIPEIAIPVLGTFALVSYI |
| | | ANKVLTVQTIDNALSKRNEKWDEVYKYIVTNWLAKVNTQIDLIRKKMKETL |
| | | ENQAEATKAIINYQYNQYTEEEKNNINFNIDDLSSKLNESINKAMININKFLN |
| | | QCSVSYLMNSMIPYGVKRLEDFDASLKDALLKYIYDNRGTLIGQVDRLKDKV |
| | | NNTLSTDIPFQLSKYVDNQRLLSTFTEYIKNSEILNNIILNLRYRDNNLIDLSGY |
| | | GAKVEVYDGVKLNDKNQFKLTSSADSKIRVTQNQNIIFNSMFLDFSVSFWI |
| | | RIPKYRNDDIQNYIHNEYTIINCMKNNSGWKISIRGNRIIWTLIDINGKTKSV |
| | | FFEYNIREDISEYINRWFFVTITNNLDNAKIYINGTLESNMDIKDIGEVIVNGE |
| | | ITFKLDGDVDRTQFIWMKYFSIFNTQLNQSNIKEIYKIQSYSEYLKDFWGNPL |
| | | MYNKEYYMFNAGNKNSYIKLVKDSSVGEILIRSKYNQNSNYINYRNLYIGEK |
| | | FIIRRESNSQSINDDIVRKEDYIHLDLVLHHEEWRVYAYKYFKEQEEKLFLSIIS |
| | | DSNEFYKTIEIKEYDEQPSYSCQLLFKKDEESTDDIGLIGIHRFYESGVLRKKYK |
| | | DYFCISKWYLKEVKRKPYKSNLGCNWQFIPKDEGWTE |
| 204 | H_{C}-BoNT/A2-WT (of | IVNTSILSIVYKKDDLIDLSRYGAKINIGDRVYYDSIDKNQIKLINLESSTIEVILK |
| | full length) | NAIVYNSMYENFSTSFWIKIPKYFSKINLNNEYTIINCIENNSGWKVSLNYGEI |
| | | IWTLQDNKQNIQRVVFKYSQMVNISDYINRWIFVTITNNRLTKSKIYINGRLI |
| | | DQKPISNLGNIHASNKIMFKLDGCRDPRRYIMIKYFNLFDKELNEKEIKDLYD |
| | | SQSNSGILKDFWGNYLQYDKPYYMLNLFDPNKYVDVNNIGIRGYMYLKGP |
| | | RGSVVTTNIYLNSTLYEGTKFIIKKYASGNEDNIVRNNDRVYINVVVKNKEYR |
| | | LATNASQAGVEKILSALEIPDVGNLSQVVVMKSKDDQGIRNKCKMNLQDN |
| | | NGNDIGFIGFHLYDNIAKLVASNWYNRQVGKASRTFGCSWEFIPVDDGW |
| | | GESSL |
| 205 | H_{C}-BoNT/A1 (of full | IINTSILNLRYESNHLIDLSRYASKINIGSKVNFDPIDKNQIQLFNLESSKIEVILK |
| | length) | NAIVYNSMYENFSTSFWIRIPKYFNSISLNNEYTIINCMENNSGWKVSLNYG |
| | | EIIWTLQDTQEIKQRVVFKYSQMINISDYINRWIFVTITNNRLNNSKIYINGR |
| | | LIDQKPISNLGNIHASNNIMFKLDGCRDTHRYIWIKYFNLFDKELNEKEIKDL |
| | | YDNQSNSGILKDFWGDYLQYDKPYYMLNLYDPNKYVDVNNVGIRGYMYL |
| | | KGPRGSVMTTNIYLNSSLYRGTKFIIKKYASGNKDNIVRNNDRVYINVVVKN |
| | | KEYRLATNASQAGVEKILSALEIPDVGNLSQVVVMKSKNDQGITNKCKMNL |
| | | QDNNGNDIGFIGFHQFNNIAKLVASNWYNRQIERSSRTLGCSWEFIPVDD |
| | | GWGERPL |
| 206 | Thrombin Cleavage | LVPRGS |
| | site | |
| 207 | TEV cleavage site | ENLYFQG |
| 208 | PreScission cleavage | LEVLFQGP |
| | site | |
| 209 | Factor Xa cleavage | IEGR |
| | site 1 | |
| 210 | Factor Xa cleavage | IDGR |
| | site 2 | |
| 211 | Enterokinase cleavage | DDDDK |
| | site 1 | |
| 212 | SUMO protease | AHREQIGG |
| | cleavage site | |
| 213 | BoNT/A1-WT with a | MPFVNKQFNYKDPVNGVDIAYIKIPNAGQMQPVKAFKIHNKIWVIPERDT |
| | thrombin cleavage | FTNPEEGDLNPPPEAKQVPVSYYDSTYLSTDNEKDNYLKGVTKLFERIYSTDL |
| | site | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNKFKDIASTLNKAKSIVGTTASLQYMK |
| | | NVFKEKYLLSEDTSGKFSVDKLKFDKLYKMLTEIYTEDNFVKFFKVLNRKTYL |
| | | NFDKAVFKINIVPKVNYTIYDGFNLRNTNLAANFNGQNTEINNMNFTKLKN |
| | | FTGLFEFYKLLCVRGIITSTSLVPRGSALNDLCIKVNNWDLFFSPSEDNFTND |
| | | LNKGEEITSDTNIEAAEENISLDLIQQYYLTFNFDNEPENISIENLSSDIIGQLEL |
| | | MPNIERFPNGKKYELDKYTMFHYLRAQEFEHGKSRIALTNSVNEALLNPSR |
| | | VYTFFSSDYVKKVNKATEAAMFLGWVEQLVYDFTDETSEVSTTDKIADITIII |
| | | PYIGPALNIGNMLYKDDFVGALIFSGAVILLEFIPEIAIPVLGTFALVSYIANKV |
| | | LTVQTIDNALSKRNEKWDEVYKYIVTNWLAKVNTQIDLIRKKMKEALENQA |
| | | EATKAIINYQYNQYTEEEKNNINFNIDDLSSKLNESINKAMININKFLNQCSV |
| | | SYLMNSMIPYGVKRLEDFDASLKDALLKYIYDNRGTLIGQVDRLKDKVNNTL |
| | | STDIPFQLSKYVDNQRLLSTFTEYIKNIINTSILNLRYESNHLIDLSRYASKINIG |
| | | SKVNFDPIDKNQIQLFNLESSKIEVILKNAIVYNSMYENFSTSFWIRIPKYFNSI |
| | | SLNNEYTIINCMENNSGWKVSLNYGEIIWTLQDTQEIKQRVVFKYSQMINIS |
| | | DYINRWIFVTITNNRLNNSKIYINGRLIDQKPISNLGNIHASNNIMFKLDGCR |
| | | DTHRYIWIKYFNLFDKELNEKEIKDLYDNQSNSGILKDFWGDYLQYDKPYY |
| | | MLNLYDPNKYVDVNNVGIRGYMYLKGPRGSVMTTNIYLNSSLYRGTKFIIK |
| | | KYASGNKDNIVRNNDRVYINVVVKNKEYRLATNASQAGVEKILSALEIPDV |
| | | GNLSQVVVMKSKNDQGITNKCKMNLQDNNGNDIGFIGFHQFNNIAKLVA |
| | | SNWYNRQIERSSRTLGCSWEFIPVDDGWGERPL |
| 214 | BoNT/A1-KR10 (K11R, | MPFVNKQFNYRDPVNGVDIAYIKIPNAGQMQPVKAFKIHNKIWVIPERDT |
| | K289R, K291R, K299R, | FTNPEEGDLNPPPEAKQVPVSYYDSTYLSTDNEKDNYLKGVTKLFERIYSTDL |
| | K335R, K337R, K343R, | GRMLLTSIVRGIPFWGGSTIDTELKVIDTNCINVIQPDGSYRSEELNLVIIGPS |
| | K375R, K381R, K415R) | ADIIQFECKSFGHEVLNLTRNGYGSTQYIRFSPDFTFGFEESLEVDTNPLLGA |
| | with a thrombin | GKFATDPAVTLAHELIHAGHRLYGIAINPNRVFKVNTNAYYEMSGLEVSFEE |
| | cleavage site | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIITSTSLVPRGSALNDLCIKVNNWDLFFSPSEDNFTN |
| | | DLNKGEEITSDTNIEAAEENISLDLIQQYYLTFNFDNEPENISIENLSSDIIGQL |
| | | ELMPNIERFPNGKKYELDKYTMFHYLRAQEFEHGKSRIALTNSVNEALLNPS |
| | | RVYTFFSSDYVKKVNKATEAAMFLGWVEQLVYDFTDETSEVSTTDKIADITII |
| | | IPYIGPALNIGNMLYKDDFVGALIFSGAVILLEFIPEIAIPVLGTFALVSYIANKV |
| | | LTVQTIDNALSKRNEKWDEVYKYIVTNWLAKVNTQIDLIRKKMKEALENQA |
| | | EATKAIINYQYNQYTEEEKNNINFNIDDLSSKLNESINKAMININKFLNQCSV |
| | | SYLMNSMIPYGVKRLEDFDASLKDALLKYIYDNRGTLIGQVDRLKDKVNNTL |
| | | STDIPFQLSKYVDNQRLLSTFTEYIKNIINTSILNLRYESNHLIDLSRYASKINIG |
| | | SKVNFDPIDKNQIQLFNLESSKIEVILKNAIVYNSMYENFSTSFWIRIPKYFNSI |
| | | SLNNEYTIINCMENNSGWKVSLNYGEIIWTLQDTQEIKQRVVFKYSQMINIS |
| | | DYINRWIFVTITNNRLNNSKIYINGRLIDQKPISNLGNIHASNNIMFKLDGCR |
| | | DTHRYIWIKYFNLFDKELNEKEIKDLYDNQSNSGILKDFWGDYLQYDKPYY |
| | | MLNLYDPNKYVDVNNVGIRGYMYLKGPRGSVMTTNIYLNSSLYRGTKFIIK |
| | | KYASGNKDNIVRNNDRVYINVVVKNKEYRLATNASQAGVEKILSALEIPDV |
| | | GNLSQVVVMKSKNDQGITNKCKMNLQDNNGNDIGFIGFHQFNNIAKLVA |
| | | SNWYNRQIERSSRTLGCSWEFIPVDDGWGERPL |
| 215 | BoNT/A2-WT with a | MPFVNKQFNYKDPVNGVDIAYIKIPNAGQMQPVKAFKIHNKIWVIPERDT |
| | thrombin cleavage | FTNPEEGDLNPPPEAKQVPVSYYDSTYLSTDNEKDNYLKGVTKLFERIYSTDL |
| | site | GRMLLTSIVRGIPFWGGSTIDTELKVIDTNCINVIQPDGSYRSEELNLVIIGPS |
| | | ADIIQFECKSFGHDVLNLTRNGYGSTQYIRFSPDFTFGFEESLEVDTNPLLGA |
| | | GKFATDPAVTLAHELIHAEHRLYGIAINPNRVFKVNTNAYYEMSGLEVSFEE |
| | | LRTFGGHDAKFIDSLQENEFRLYYYNKFKDVASTLNKAKSIIGTTASLQYMK |
| | | NVFKEKYLLSEDTSGKFSVDKLKFDKLYKMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFTSLVPRGSALNDLCIKVNNWDLFFSPSEDNFTNDL |
| | | DKVEEITADTNIEAAEENISLDLIQQYYLTFDFDNEPENISIENLSSDIIGQLEP |
| | | MPNIERFPNGKKYELDKYTMFHYLRAQEFEHGDSRIILTNSAEEALLKPNVA |
| | | YTFFSSKYVKKINKAVEAFMFLNWAEELVYDFTDETNEVTTMDKIADITIIVP |
| | | YIGPALNIGNMLSKGEFVEAIIFTGVVAMLEFIPEYALPVFGTFAIVSYIANKV |
| | | LTVQTINNALSKRNEKWDEVYKYTVTNWLAKVNTQIDLIREKMKKALENQ |
| | | AEATKAIINYQYNQYTEEEKNNINFNIDDLSSKLNESINSAMININKFLDQCS |
| | | VSYLMNSMIPYAVKRLKDFDASVRDVLLKYIYDNRGTLVLQVDRLKDEVNN |
| | | TLSADIPFQLSKYVDNKKLLSTFTEYIKNIVNTSILSIVYKKDDLIDLSRYGAKINI |
| | | GDRVYYDSIDKNQIKLINLESSTIEVILKNAIVYNSMYENFSTSFWIKIPKYFSKI |
| | | NLNNEYTIINCIENNSGWKVSLNYGEIIWTLQDNKQNIQRVVFKYSQMVNI |
| | | SDYINRWIFVTITNNRLTKSKIYINGRLIDQKPISNLGNIHASNKIMFKLDGCR |
| | | DPRRYIMIKYFNLFDKELNEKEIKDLYDSQSNSGILKDFWGNYLQYDKPYYM |
| | | LNLFDPNKYVDVNNIGIRGYMYLKGPRGSVVTTNIYLNSTLYEGTKFIIKKYA |
| | | SGNEDNIVRNNDRVYINVVVKNKEYRLATNASQAGVEKILSALEIPDVGNLS |
| | | QVVVMKSKDDQGIRNKCKMNLQDNNGNDIGFIGFHLYDNIAKLVASNWY |
| | | NRQVGKASRTFGCSWEFIPVDDGWGESSL |
| 216 | BoNT/A2-KR7 (K11R, | MPFVNKQFNYRDPVNGVDIAYIKIPNAGQMQPVKAFKIHNKIWVIPERDT |
| | K289R, K291R, K299R, | FTNPEEGDLNPPPEAKQVPVSYYDSTYLSTDNEKDNYLKGVTKLFERIYSTDL |
| | K335R, K337R, K343R) | GRMLLTSIVRGIPFWGGSTIDTELKVIDTNCINVIQPDGSYRSEELNLVIIGPS |
| | with a thrombin | ADIIQFECKSFGHDVLNLTRNGYGSTQYIRFSPDFTFGFEESLEVDTNPLLGA |
| | cleavage site | GKFATDPAVTLAHELIHAEHRLYGIAINPNRVFKVNTNAYYEMSGLEVSFEE |
| | | LRTFGGHDAKFIDSLQENEFRLYYYNRFRDVASTLNRAKSIIGTTASLQYMK |
| | | NVFKEKYLLSEDTSGKFSVDRLRFDKLYRMLTEIYTEDNFVNFFKVINRKTYL |
| | | NFDKAVFRINIVPDENYTIKDGFNLKGANLSTNFNGQNTEINSRNFTRLKNF |
| | | TGLFEFYKLLCVRGIIPFTSLVPRGSALNDLCIKVNNWDLFFSPSEDNFTNDL |
| | | DKVEEITADTNIEAAEENISLDLIQQYYLTFDFDNEPENISIENLSSDIIGQLEP |
| | | MPNIERFPNGKKYELDKYTMFHYLRAQEFEHGDSRIILTNSAEEALLKPNVA |
| | | YTFFSSKYVKKINKAVEAFMFLNWAEELVYDFTDETNEVTTMDKIADITIIVP |
| | | YIGPALNIGNMLSKGEFVEAIIFTGVVAMLEFIPEYALPVFGTFAIVSYIANKV |
| | | LTVQTINNALSKRNEKWDEVYKYTVTNWLAKVNTQIDLIREKMKKALENQ |
| | | AEATKAIINYQYNQYTEEEKNNINFNIDDLSSKLNESINSAMININKFLDQCS |
| | | VSYLMNSMIPYAVKRLKDFDASVRDVLLKYIYDNRGTLVLQVDRLKDEVNN |
| | | TLSADIPFQLSKYVDNKKLLSTFTEYIKNIVNTSILSIVYKKDDLIDLSRYGAKINI |
| | | GDRVYYDSIDKNQIKLINLESSTIEVILKNAIVYNSMYENFSTSFWIKIPKYFSKI |
| | | NLNNEYTIINCIENNSGWKVSLNYGEIIWTLQDNKQNIQRVVFKYSQMVNI |
| | | SDYINRWIFVTITNNRLTKSKIYINGRLIDQKPISNLGNIHASNKIMFKLDGCR |
| | | DPRRYIMIKYFNLFDKELNEKEIKDLYDSQSNSGILKDFWGNYLQYDKPYYM |
| | | LNLFDPNKYVDVNNIGIRGYMYLKGPRGSVVTTNIYLNSTLYEGTKFIIKKYA |
| | | SGNEDNIVRNNDRVYINVVVKNKEYRLATNASQAGVEKILSALEIPDVGNLS |
| | | QVVVMKSKDDQGIRNKCKMNLQDNNGNDIGFIGFHLYDNIAKLVASNWY |
| | | NRQVGKASRTFGCSWEFIPVDDGWGESSL |
| 217 | LC-HN BoNT/A1 KR10 - | MPFVNKQFNYRDPVNGVDIAYIKIPNAGQMQPVKAFKIHNKIWVIPERDT |
| | H_{C}/B w/ thrombin | FTNPEEGDLNPPPEAKQVPVSYYDSTYLSTDNEKDNYLKGVTKLFERIYSTDL |
| | cleavage site | GRMLLTSIVRGIPFWGGSTIDTELKVIDTNCINVIQPDGSYRSEELNLVIIGPS |
| | | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIIPFTSLVPRGSALNDLCIKVNNWDLFFSPSEDNFTN |
| | | DLNKGEEITSDTNIEAAEENISLDLIQQYYLTFNFDNEPENISIENLSSDIIGQL |
| | | ELMPNIERFPNGKKYELDKYTMFHYLRAQEFEHGKSRIALTNSVNEALLNPS |
| | | RVYTFFSSDYVKKVNKATEAAMFLGWVEQLVYDFTDETSEVSTTDKIADITII |
| | | IPYIGPALNIGNMLYKDDFVGALIFSGAVILLEFIPEIAIPVLGTFALVSYIANKV |
| | | LTVQTIDNALSKRNEKWDEVYKYIVTNWLAKVNTQIDLIRKKMKETLENQA |
| | | EATKAIINYQYNQYTEEEKNNINFNIDDLSSKLNESINKAMININKFLNQCSV |
| | | SYLMNSMIPYGVKRLEDFDASLKDALLKYIYDNRGTLIGQVDRLKDKVNNTL |
| | | STDIPFQLSKYVDNQRLLSTFTEYIKNSEILNNIILNLRYRDNNLIDLSGYGAKV |
| | | EVYDGVKLNDKNQFKLTSSADSKIRVTQNQNIIFNSMFLDFSVSFWIRIPKY |
| | | RNDDIQNYIHNEYTIINCMKNNSGWKISIRGNRIIWTLIDINGKTKSVFFEYN |
| | | IREDISEYINRWFFVTITNNLDNAKIYINGTLESNMDIKDIGEVIVNGEITFKL |
| | | DGDVDRTQFIWMKYFSIFNTQLNQSNIKEIYKIQSYSEYLKDFWGNPLMYN |
| | | KEYYMFNAGNKNSYIKLVKDSSVGEILIRSKYNQNSNYINYRNLYIGEKFIIRR |
| | | ESNSQSINDDIVRKEDYIHLDLVLHHEEWRVYAYKYFKEQEEKLFLSIISDSNE |
| | | FYKTIEIKEYDEQPSYSCQLLFKKDEESTDDIGLIGIHRFYESGVLRKKYKDYFCI |
| | | SKWYLKEVKRKPYKSNLGCNWQFIPKDEGWTE |
| 218 | BoNT/A1 - KR10/ | MPFVNKQFNYRDPVNGVDIAYIKIPNAGQMQPVKAFKIHNKIWVIPERDT |
| | R1156M (of full | FTNPEEGDLNPPPEAKQVPVSYYDSTYLSTDNEKDNYLKGVTKLFERIYSTDL |
| | length) w/ thrombin | GRMLLTSIVRGIPFWGGSTIDTELKVIDTNCINVIQPDGSYRSEELNLVIIGPS |
| | cleavage site | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIIPFTSLVPRGSALNDLCIKVNNWDLFFSPSEDNFTN |
| | | DLNKGEEITSDTNIEAAEENISLDLIQQYYLTFNFDNEPENISIENLSSDIIGQL |
| | | ELMPNIERFPNGKKYELDKYTMFHYLRAQEFEHGKSRIALTNSVNEALLNPS |
| | | RVYTFFSSDYVKKVNKATEAAMFLGWVEQLVYDFTDETSEVSTTDKIADITII |
| | | IPYIGPALNIGNMLYKDDFVGALIFSGAVILLEFIPEIAIPVLGTFALVSYIANKV |
| | | LTVQTIDNALSKRNEKWDEVYKYIVTNWLAKVNTQIDLIRKKMKETLENQA |
| | | EATKAIINYQYNQYTEEEKNNINFNIDDLSSKLNESINKAMININKFLNQCSV |
| | | SYLMNSMIPYGVKRLEDFDASLKDALLKYIYDNRGTLIGQVDRLKDKVNNTL |
| | | STDIPFQLSKYVDNQRLLSTFTEYIKNIINTSILNLRYESNHLIDLSRYASKINIG |
| | | SKVNFDPIDKNQIQLFNLESSKIEVILKNAIVYNSMYENFSTSFWIRIPKYFNSI |
| | | SLNNEYTIINCMENNSGWKVSLNYGEIIWTLQDTQEIKQRVVFKYSQMINIS |
| | | DYINRWIFVTITNNRLNNSKIYINGRLIDQKPISNLGNIHASNNIMFKLDGCR |
| | | DTHRYIWIKYFNLFDKELNEKEIKDLYDNQSNSGILKDFWGDYLQYDKPYY |
| | | MLNLYDPNKYVDVNNVGIRGYMYLKGPRGSVMTTNIYLNSSLYMGTKFIIK |
| | | KYASGNKDNIVRNNDRVYINVVVKNKEYRLATNASQAGVEKILSALEIPDV |
| | | GNLSQVVVMKSKNDQGITNKCKMNLQDNNGNDIGFIGFHQFNNIAKLVA |
| | | SNWYNRQIERSSRTLGCSWEFIPVDDGWGERPL |
| 219 | BoNT/A1 - KR10/ | MPFVNKQFNYRDPVNGVDIAYIKIPNAGQMQPVKAFKIHNKIWVIPERDT |
| | T1232R (of full length) | FTNPEEGDLNPPPEAKQVPVSYYDSTYLSTDNEKDNYLKGVTKLFERIYSTDL |
| | w/ thrombin cleavage | GRMLLTSIVRGIPFWGGSTIDTELKVIDTNCINVIQPDGSYRSEELNLVIIGPS |
| | site | ADIIQFECKSFGHEVLNLTRNGYGSTQYIRFSPDFTFGFEESLEVDTNPLLGA |
| | | GKFATDPAVTLAHELIHAGHRLYGIAINPNRVFKVNTNAYYEMSGLEVSFEE |
| | | LRTFGGHDAKFIDSLQENEFRLYYYNRFRDIASTLNRAKSIVGTTASLQYMK |
| | | NVFKEKYLLSEDTSGKFSVDRLRFDKLYRMLTEIYTEDNFVKFFKVLNRKTYL |
| | | NFDKAVFRINIVPRVNYTIYDGFNLRNTNLAANFNGQNTEINNMNFTRLK |
| | | NFTGLFEFYKLLCVRGIIPFTSLVPRGSALNDLCIKVNNWDLFFSPSEDNFTN |
| | | DLNKGEEITSDTNIEAAEENISLDLIQQYYLTFNFDNEPENISIENLSSDIIGQL |
| | | ELMPNIERFPNGKKYELDKYTMFHYLRAQEFEHGKSRIALTNSVNEALLNPS |
| | | RVYTFFSSDYVKKVNKATEAAMFLGWVEQLVYDFTDETSEVSTTDKIADITII |
| | | IPYIGPALNIGNMLYKDDFVGALIFSGAVILLEFIPEIAIPVLGTFALVSYIANKV |
| | | LTVQTIDNALSKRNEKWDEVYKYIVTNWLAKVNTQIDLIRKKMKETLENQA |
| | | EATKAIINYQYNQYTEEEKNNINFNIDDLSSKLNESINKAMININKFLNQCSV |
| | | SYLMNSMIPYGVKRLEDFDASLKDALLKYIYDNRGTLIGQVDRLKDKVNNTL |
| | | STDIPFQLSKYVDNQRLLSTFTEYIKNIINTSILNLRYESNHLIDLSRYASKINIG |
| | | SKVNFDPIDKNQIQLFNLESSKIEVILKNAIVYNSMYENFSTSFWIRIPKYFNSI |
| | | SLNNEYTIINCMENNSGWKVSLNYGEIIWTLQDTQEIKQRVVFKYSQMINIS |
| | | DYINRWIFVTITNNRLNNSKIYINGRLIDQKPISNLGNIHASNNIMFKLDGCR |
| | | DTHRYIWIKYFNLFDKELNEKEIKDLYDNQSNSGILKDFWGDYLQYDKPYY |
| | | MLNLYDPNKYVDVNNVGIRGYMYLKGPRGSVMTTNIYLNSSLYRGTKFIIK |
| | | KYASGNKDNIVRNNDRVYINVVVKNKEYRLATNASQAGVEKILSALEIPDV |
| | | GNLSQVVVMKSKNDQGIRNKCKMNLQDNNGNDIGFIGFHQFNNIAKLVA |
| | | SNWYNRQIERSSRTLGCSWEFIPVDDGWGERPL |

### REFERENCES: EXAMPLES

1. Schiavo, G., Matteoli, M. & Montecucco, C. Neurotoxins affecting neuroexocytosis. Physiol Rev 80, 717-766 (2000).
2. Dong, M., Masuyer, G. & Stenmark, P. Botulinum and Tetanus Neurotoxins. Annu Rev Biochem 88, 811-837 (2019).
3. Johnson, E. A. Clostridial toxins as therapeutic agents: benefits of nature's most toxic proteins. Annu Rev Microbiol 53, 551-575 (1999).
4. Aoki, K. R. Botulinum toxin: a successful therapeutic protein. Curr Med Chem 11, 3085-3092 (2004).
5. Montecucco, C. & Molgo, J. Botulinal neurotoxins: revival of an old killer. Curr Opin Pharmacol 5, 274-279 (2005).
6. Lange, O., Bigalke, H., Dengler, R., Wegner, F., deGroot, M. & Wohlfarth, K. Neutralizing antibodies and secondary therapy failure after treatment with botulinum toxin type A: much ado about nothing? Clin Neuropharmacol 32, 213-218 (2009).
7 Chapman, M. A., Barron, R., Tanis, D. C., Gill, C. E. & Charles, P. D. Comparison of botulinum neurotoxin preparations for the treatment of cervical dystonia. Clin Ther 29, 1325-1337 (2007).
8. Cote, T. R., Mohan, A. K., Polder, J. A., Walton, M. K. & Braun, M. M. Botulinum toxin type A injections: adverse events reported to the US Food and Drug Administration in therapeutic and cosmetic cases. J Am Acad Dermatol 53, 407-415 (2005).
9. Tao, L., Peng, L., Berntsson, R. P., Liu, S. M., Park, S., Yu, F., Boone, C., Palan, S., Beard, M., Chabrier, P. E., Stenmark, P., Krupp, J. & Dong, M. Engineered botulinum neurotoxin B with improved efficacy for targeting human receptors. Nat Commun 8, 53 (2017).
10. Elliott, M., Favre-Guilmard, C., Liu, S. M., Maignel, J., Masuyer, G., Beard, M., Boone, C., Carre, D., Kalinichev, M., Lezmi, S., Mir, I., Nicoleau, C., Palan, S., Perier, C., Raban, E., Zhang, S., Dong, M., Stenmark, P. & Krupp, J. Engineered botulinum neurotoxin B with improved binding to human receptors has enhanced efficacy in preclinical models. Sci Adv 5, eaau7196 (2019).
11. Pirazzini, M., Henke, T., Rossetto, O., Mahrhold, S., Krez, N., Rummel, A., Montecucco, C. & Binz, T. Neutralisation of specific surface carboxylates speeds up translocation of botulinum neurotoxin type B enzymatic domain. FEBS Lett 587, 3831-3836 (2013).
12. Guo, J., Pan, X., Zhao, Y. & Chen, S. Engineering Clostridia Neurotoxins with elevated catalytic activity. Toxicon 74, 158-166 (2013).
13. de la Paz, M. L., Scheps, D., Jurk, M., Hofmann, F. & Frevert, J. Rational design of botulinum neurotoxin A1 mutants with improved oxidative stability. Toxicon 147, 54-57 (2018).
14. Tsai, Y. C., Maditz, R., Kuo, C.-I., Fishman, P. S., Shoemaker, C. B., Oyler, G. A. & Weissman, A. M. Targeting botulinum neurotoxin persistence by the ubiquitin-proteasome system. Proceedings of the National Academy of Sciences 107, 16554-16559 (2010).
15. Li, L., Vorobyov, I. & Allen, T. W. The different interactions of lysine and arginine side chains with lipid membranes. The Journal of Physical Chemistry B 117, 11906-11920 (2013).
16. Åmand, H. L., Fant, K., Norden, B. & Esbjorner, E. K. Stimulated endocytosis in penetratin uptake: effect of arginine and lysine. Biochemical and biophysical research communications 371, 621-625 (2008).
17. Zou, G., de Leeuw, E., Li, C., Pazgier, M., Li, C., Zeng, P., Lu, W.-Y., Lubkowski, J. & Lu, W. Toward understanding the cationicity of defensins ARG and LYS versus their noncoded analogs. Journal of biological chemistry 282, 19653-19665 (2007).
18. Peck, M. W., Smith, T. J., Anniballi, F., Austin, J. W., Bano, L., Bradshaw, M., Cuervo, P., Cheng, L. W., Derman, Y., Dorner, B. G., Fisher, A., Hill, K. K., Kalb, S. R., Korkeala, H., Lindstrom, M., Lista, F., Luquez, C., Mazuet, C., Pirazzini, M., Popoff, M. R., Rossetto, O., Rummel, A., Sesardic, D., Singh, B. R. & Stringer, S. C. Historical Perspectives and Guidelines for Botulinum Neurotoxin Subtype Nomenclature. Toxins (Basel) 9(2017).
19. Sternke, M., Tripp, K. W. & Barrick, D. Consensus sequence design as a general strategy to create hyperstable, biologically active proteins. Proceedings of the National Academy of Sciences 116, 11275-11284 (2019).
20. Breidenbach, M. A. & Brunger, A. T. Substrate recognition strategy for botulinum neurotoxin serotype A. Nature 432, 925-929 (2004).
21. Zhang, S., Masuyer, G., Zhang, J., Shen, Y., Lundin, D., Henriksson, L., Miyashita, S. I., Martinez-Carranza, M., Dong, M. & Stenmark, P. Identification and characterization of a novel botulinum neurotoxin. Nat Commun 8, 14130 (2017).
22. Popp, M. W., Antos, J. M., Grotenbreg, G. M., Spooner, E. & Ploegh, H. L. Sortagging: a versatile method for protein labeling. Nat Chem Biol 3, 707-708 (2007).
23. Aoki, K. R. A comparison of the safety margins of botulinum neurotoxin serotypes A, B, and F in mice. Toxicon 39, 1815-1820 (2001).
24. Wang, J., Zurawski, T. H., Bodeker, M. O., Meng, J., Boddul, S., Aoki, K. R. & Dolly, J. O. Longer-acting and highly potent chimaeric inhibitors of excessive exocytosis created with domains from botulinum neurotoxin A and B. Biochem J 444, 59-67 (2012).
25. Arnon, S. S., Schechter, R., Inglesby, T. V., Henderson, D. A., Bartlett, J. G., Ascher, M. S., Eitzen, E., Fine, A. D., Hauer, J., Layton, M., Lillibridge, S., Osterholm, M. T., O'Toole, T., Parker, G., Perl, T. M., Russell, P. K., Swerdlow, D. L. & Tonat, K. Botulinum toxin as a biological weapon: medical and public health management. Jama 285, 1059-1070 (2001).
26. Gustafsson, R., Zhang, S., Masuyer, G., Dong, M. & Stenmark, P. Crystal structure of botulinum neurotoxin A2 in complex with the human protein receptor SV2C reveals plasticity in receptor binding. Toxins 10, 153 (2018).
27. Peng, L., Tepp, W. H., Johnson, E. A. & Dong, M. Botulinum neurotoxin D uses synaptic vesicle protein SV2 and gangliosides as receptors. PLoS Pathog 7, e1002008 (2011).

What is claimed is:

1. A modified Clostridial Botulinum neurotoxin (BoNT) polypeptide comprising a modified protease domain of Clostridial Botulinum serotype A1 (BoNT/A1) comprising one or more arginine substitutions at positions corresponding to K11, K41, K212, K272, K289, K291, K299, K318, K335, K337, K340, K343, K356, K375, K381, Y387, M411 and K415 in SEQ ID NO: 1.

2. The modified BoNT polypeptide of claim 1, wherein the modified protease domain comprises one or more arginine substitutions at positions corresponding to K11, K335, K337, K343, K375, and K415 in SEQ ID NO: 1.

3. The modified BoNT polypeptide of claim 1, wherein the modified protease domain comprises one or more arginine substitutions at positions corresponding to K41, K318, and K340 in SEQ ID NO: 1.

4. The modified BoNT polypeptide of claim 1, wherein the modified protease domain comprises one or more arginine substitutions at positions corresponding to K289, K291, K299, and K381 in SEQ ID NO: 1.

5. The modified BoNT polypeptide of claim 1, wherein the modified protease domain comprises one or more arginine substitutions at positions corresponding to K212, K272, and K356 in SEQ ID NO: 1.

6. The modified BoNT polypeptide of claim 1, wherein the modified protease domain comprises one or more arginine substitutions at positions corresponding to K11, K41, K318, K335, K337, K340, K343, K375, and K415 in SEQ ID NO: 1.

7. The modified BoNT polypeptide of claim 1, wherein the modified protease domain comprises one or more arginine substitutions at positions corresponding to K11, K289, K291, K299, K335, K337, K343, K375, K381, and K415 in SEQ ID NO: 1.

8. The modified BoNT polypeptide of claim 7, wherein the modified protease domain comprises arginine substitutions at positions corresponding to K11, K289, K291, K299, K335, K337, K343, K375, K381, and K415 in SEQ ID NO: 1.

9. The modified BoNT polypeptide of claim 7 or claim 8, wherein the modified protease domain further comprises one or more arginine substitutions at positions corresponding to K41, K212, K272, K318, K340, and K356 in SEQ ID NO: 1.

10. The modified BoNT polypeptide of 1, wherein the modified protease domain comprises one or more arginine substitutions at positions corresponding to K11, K41, K289, K291, K299, K318, K335, K337, K340, K343, K375, K381, and K415 in SEQ ID NO: 1.

11. The modified BoNT polypeptide of 10, wherein the modified protease domain comprises arginine substitutions at positions corresponding to K11, K41, K289, K291, K299, K318, K335, K337, K340, K343, K375, K381 and K415 in SEQ ID NO: 1.

12. The modified BoNT polypeptide of 1, wherein the modified protease domain comprises one or more arginine substitutions at positions corresponding to K11, K41, K212, K289, K291, K299, K318, K335, K337, K340, K343, K356, K375, K381, and K415 in SEQ ID NO: 1.

13. The modified BoNT polypeptide of 12, wherein the modified protease domain comprises arginine substitutions at positions corresponding to K11, K41, K212, K289, K291, K299, K318, K335, K337, K340, K343, K356, K375, K381, and K415 in SEQ ID NO: 1.

14. The modified BoNT polypeptide of 1, wherein the modified protease domain comprises one or more arginine substitutions at positions corresponding to K11, K41, K212, K272, K289, K291, K299, K318, K335, K337, K340, K343, K356, K375, K381, and K415 in SEQ ID NO: 1.

15. The modified BoNT polypeptide of 1, wherein the modified protease domain comprises arginine substitutions at positions corresponding to K11, K41, K212, K272, K289, K291, K299, K318, K335, K337, K340, K343, K356, K375, K381, and K415 in SEQ ID NO: 1.

16. The modified BoNT polypeptide of any one of claims 1-15, wherein the modified protease domain comprises an arginine substitution of K11R in SEQ ID NO: 1.

17. The modified BoNT polypeptide of any one of claims 1-16, wherein the modified protease domain comprises an arginine substitution of K41R in SEQ ID NO: 1.

18. The modified BoNT polypeptide of any one of claims 1-17, wherein the modified protease domain comprises an arginine substitution of K212R in SEQ ID NO: 1.

19. The modified BoNT polypeptide of any one of claims 1-18, wherein the modified protease domain comprises an arginine substitution of K272R in SEQ ID NO: 1.

20. The modified BoNT polypeptide of any one of claims 1-19, wherein the modified protease domain comprises an arginine substitution of K289R in SEQ ID NO: 1.

21. The modified BoNT polypeptide of any one of claims 1-20, wherein the modified protease domain comprises an arginine substitution of K291R in SEQ ID NO: 1.

22. The modified BoNT polypeptide of any one of claims 1-21, wherein the modified protease domain comprises an arginine substitution of K299R in SEQ ID NO: 1.

23. The modified BoNT polypeptide of any one of claims 1-22, wherein the modified protease domain comprises an arginine substitution of K318R in SEQ ID NO: 1.

24. The modified BoNT polypeptide of any one of claims 1-23, wherein the modified protease domain comprises an arginine substitution of K335R in SEQ ID NO: 1.

25. The modified BoNT polypeptide of any one of claims 1-24, wherein the modified protease domain comprises an arginine substitution of K337R in SEQ ID NO: 1.

26. The modified BoNT polypeptide of any one of claims 1-25, wherein the modified protease domain comprises an arginine substitution of K340R in SEQ ID NO: 1.

27. The modified BoNT polypeptide of any one of claims 1-26, wherein the modified protease domain comprises an arginine substitution of K343R in SEQ ID NO: 1.

28. The modified BoNT polypeptide of any one of claims 1-27, wherein the modified protease domain comprises an arginine substitution of K356R in SEQ ID NO: 1.

29. The modified BoNT polypeptide of any one of claims 1-28, wherein the modified protease domain comprises an arginine substitution of K375R in SEQ ID NO: 1.

30. The modified BoNT polypeptide of any one of claims 1-29, wherein the modified protease domain comprises an arginine substitution of K381R in SEQ ID NO: 1.

31. The modified BoNT polypeptide of any one of claims 1-30, wherein the modified protease domain comprises an arginine substitution of Y387R in SEQ ID NO: 1.

32. The modified BoNT polypeptide of any one of claims 1-31, wherein the modified protease domain comprises an arginine substitution of M411R in SEQ ID NO: 1.

33. The modified BoNT polypeptide of any one of claims 1-32, wherein the modified protease domain comprises an arginine substitution of K415R in SEQ ID NO: 1.

34. The modified BoNT polypeptide of any one of claims 1-33, wherein the modified protease domain comprises an amino acid sequence that is at least 80% identical to the amino acid sequence of SEQ ID NO: 88-120, optionally wherein the modified protease binding domain comprises the amino acid sequence of any one of SEQ ID NOs: 88-120.

35. The modified BoNT polypeptide of any one of claims 1-34, further comprising a translocation domain from BoNT/A1.

36. The modified BoNT polypeptide of any one of claims 1-35, wherein the modified BoNT polypeptide comprises an amino acid sequence that is at least 80% identical to the amino acid sequence of SEQ ID NO: 45-77, optionally wherein the modified BoNT polypeptide comprises the amino acid sequence of any one of SEQ ID NOs: 45-77.

37. The modified BoNT polypeptide of claim 36, further comprising a receptor binding domain of BoNT/A1.

38. The modified BoNT polypeptide of claim 37, wherein the modified BoNT polypeptide comprises an amino acid sequence that is at least 80% identical to the amino acid sequence of any one of SEQ ID NOs: 2-34, optionally wherein the modified BoNT polypeptide comprises the amino acid sequence of any one of SEQ ID NOs: 2-34.

39. The modified BoNT polypeptide of claim 37, wherein the receptor binding domain of BoNT/A1 comprises one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1, optionally wherein the one or more amino acid substitutions correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1.

40. The modified BoNT polypeptide of claim 39, wherein the receptor binding domain of BoNT/A1 comprises an amino acid substitution at a position corresponding to 1156 or 1232 in SEQ ID NO: 1, optionally wherein the amino acid substitution corresponds to R1156M or T1232R in SEQ ID NO: 1.

41. The modified BoNT polypeptide of claim 39 or claim 40, wherein the modified BoNT polypeptide comprises an amino acid sequence that is at least 80% identical to the amino acid sequence of any one of SEQ ID NOs: 131-165 fused to any one of SEQ ID NOs: 45-77.

42. The modified BoNT polypeptide of claim 41, wherein the modified BoNT polypeptide comprises an amino acid sequence that is at least 80% identical to the amino acid sequence of SEQ ID No: 177 or SEQ ID NO: 178, optionally wherein the modified BoNT polypeptide comprises the amino acid sequence of SEQ ID NO: 177 or SEQ ID NO: 178.

43. The modified BoNT polypeptide of claim 36, further comprising a receptor binding domain from BoNT/A2.

44. The modified BoNT polypeptide of claim 43, wherein the receptor binding domain comprises one or more amino acid substitutions at positions corresponding to 915, 923, 1090, 1103, 1117, 1156, 1170, 1227, 1254, 1255, or 1256 in SEQ ID NO: 35, optionally wherein the one or more amino acid substitutions correspond to one or more of K915Q, T923K, 51090N, N1103D, F1117Y, E1156M, E1170K, D1227N, L1254Q, Y1255G, or D1256N in SEQ ID NO: 35.

45. The modified BoNT polypeptide of claim 44, wherein the modified BoNT polypeptide comprises an amino acid sequence that is at least 80% identical to the amino acid sequence of SEQ ID NO: 166-176 or 204 fused to any one of SEQ ID NO: 45-77.

46. The modified BoNT polypeptide of claim 45, wherein the modified BoNT polypeptide comprises the amino acid sequence of SEQ ID NO: 166-176 or 204 fused to any one of SEQ ID NO: 45-77.

47. The modified BoNT polypeptide of any one of claims 1-35, further comprising a receptor binding domain from a second BoNT, optionally wherein the second BoNT is of serotype B, C, D, E, F, G, H, or En.

48. The modified BoNT polypeptide of claim 47, wherein the modified BoNT polypeptide comprises an amino acid sequence that is at least 80% identical to the amino acid sequence of any one of SEQ ID NO: 179-186 fused to any one of SEQ ID NO: 45-77.

49. The modified BoNT polypeptide of claim 47 or claim 48, wherein the modified BoNT polypeptide comprises the amino acid sequence of SEQ ID NO: 179-186 fused to any one of SEQ ID NO: 45-77.

50. The modified BoNT polypeptide of any one of claims 47-49, wherein the modified BoNT polypeptide comprises the amino acid sequence of SEQ ID NO: 203.

51. The modified BoNT polypeptide of any one of claims 35-50 further comprising a modified linker region.

52. The modified BoNT polypeptide of claim 51, wherein the modified linker region comprises a protease cleavage site.

53. The modified BoNT polypeptide of claim 52, wherein the protease cleavage site comprises the amino acid sequence of any one of SEQ ID NO: 206-212.

54. A modified Clostridial Botulinum neurotoxin (BoNT) polypeptide comprising a modified protease domain of Clostridial Botulinum serotype A2 (BoNT/A2) comprising one or more arginine substitutions at positions corresponding to K11, K289, K291, K299, K335, K337, and K343 in SEQ ID NO: 35.

55. A modified Clostridial Botulinum neurotoxin (BoNT) polypeptide of claim B1, wherein the modified protease domain of Clostridial Botulinum serotype A2 (BoNT/A2) comprises arginine substitutions at positions corresponding to K11, K289, K291, K299, K335, K337, and K343 in SEQ ID NO: 35.

56. The modified BoNT polypeptide of claim 54 or claim 55, wherein the modified protease domain comprises an arginine substitution of K11R in SEQ ID NO: 35.

57. The modified BoNT polypeptide of any one of claims 54-56, wherein the modified protease domain comprises an arginine substitution of K289R in SEQ ID NO: 35.

58. The modified BoNT polypeptide of any one of claims 54-57, wherein the modified protease domain comprises an arginine substitution of K291R in SEQ ID NO: 35.

59. The modified BoNT polypeptide of any one of claims 54-58, wherein the modified protease domain comprises an arginine substitution of K299R in SEQ ID NO: 35.

60. The modified BoNT polypeptide of any one of claims 54-59, wherein the modified protease domain comprises an arginine substitution of K335R in SEQ ID NO: 35.

61. The modified BoNT polypeptide of any one of claims 54-60, wherein the modified protease domain comprises an arginine substitution of K337R in SEQ ID NO: 35.

62. The modified BoNT polypeptide of any one of claims 54-61, wherein the modified protease domain comprises an arginine substitution of K343R in SEQ ID NO: 35.

63. The modified BoNT polypeptide of any one of claims 54-62, wherein the modified protease domain comprises an amino acid sequence that is at least 80% identical to the amino acid sequence of SEQ ID NO: 122-129. optionally wherein the modified protease binding domain comprises the amino acid sequence of any one of SEQ ID NOs: 122-129.

64. The modified BoNT polypeptide of any one of claims 54-63, further comprising a translocation domain from BoNT/A2.

65. The modified BoNT polypeptide of any one of claim 64, wherein the modified BoNT polypeptide comprises an amino acid sequence that is at least 80% identical to the amino acid sequence of SEQ ID NO: 79-86, optionally wherein the modified BoNT polypeptide comprises the amino acid sequence of any one of SEQ ID NOs: 79-86.

66. The modified BoNT polypeptide of claim 65, further comprising a receptor binding domain of BoNT/A1.

67. The modified BoNT polypeptide of claim 66, wherein the modified BoNT polypeptide comprises an amino acid sequence that is at least 80% identical to the amino acid sequence of any one of SEQ ID NOs: 36-43, optionally wherein the modified BoNT polypeptide comprises the amino acid sequence of any one of SEQ ID NOs: 36-43.

68. The modified BoNT polypeptide of claim 66, wherein the receptor binding domain of BoNT/A1 comprises one or more amino acid substitutions at positions corresponding to 917, 953, 954, 955, 957, 968, 1025, 1026, 1052, 1062, 1063, 1064, 1065, 1066, 1145, 1156, 1232, 1272, 1278, 1288, 1289, 1292, 1294, and 1295 in SEQ ID NO: 1, optionally wherein the one or more amino acid substitutions correspond to one or more of F917R or F917K; F953H or F953Y; N954S; S955K; S957N, S957Q or S957Y; M968I; N1025T; N1026K; N1052K; D1062E; T1063P; H1064R or H1064Q; R1065N; Y1066R or Y1066K; T1145Y; R1156M or R1156I; T1232R or T1232K; E1272G; L1278F, L1278Y or L1278W; D1288E or D1288N; D1289Y; G1292R or G1292K; R1294S or R1294T; and P1295S or P1295T in SEQ ID NO: 1.

69. The modified BoNT polypeptide of claim 68, wherein the receptor binding domain of BoNT/A1 comprises an amino acid substitution at a position corresponding to 1156 or 1232 in SEQ ID NO: 1, optionally wherein the amino acid substitution corresponds to R1156M or T1232R in SEQ ID NO: 1.

70. The modified BoNT polypeptide of claim 68 or claim 69, wherein the modified BoNT polypeptide comprises an amino acid sequence that is at least 80% identical to the amino acid sequence of any one of SEQ ID NOs: 131-165 or 205 fused to any one of SEQ ID NOs: 79-86, optionally wherein the modified BoNT polypeptide comprises an amino acid sequence of any one of SEQ ID NOs: 131-165 or 205 fused to any one of SEQ ID NOs: 79-86.

71. The modified BoNT polypeptide of claim 65, further comprising a receptor binding domain from BoNT/A2.

72. The modified BoNT polypeptide of claim 71, wherein the receptor binding domain comprises one or more amino acid substitutions at positions corresponding to 915, 923, 1090, 1103, 1117, 1156, 1170, 1227, 1254, 1255, or 1256 in SEQ ID NO: 35, optionally wherein one or more the amino acid substitutions corresponds to one or more of K915Q, T923K, 51090N, N1103D, F1117Y, E1156M, E1170K, D1227N, L1254Q, Y1255G, or D1256N in SEQ ID NO: 35.

73. The modified BoNT polypeptide of claim 72, wherein the modified BoNT polypeptide comprises an amino acid sequence that is at least 80% identical to the amino acid sequence of SEQ ID NO: 166-176 fused to any one of SEQ ID NO: 79-86, optionally wherein the modified BoNT polypeptide comprises the amino acid sequence of any one of SEQ ID NO: 166-176 fused to any one of SEQ ID NO: 79-86.

74. The modified BoNT polypeptide of any one of claims 54-73, further comprising a receptor binding domain from a second BoNT, optionally wherein the second BoNT is of serotype B, C, D, E, F, G, H, or En.

75. The modified BoNT polypeptide of claim 74, wherein the modified BoNT polypeptide comprises an amino acid sequence that is at least 80% identical to the amino acid sequence of any one of SEQ ID NO: 179-186 fused to any one of SEQ ID NO: 79-86.

76. The modified BoNT polypeptide of claim 75, wherein the modified BoNT polypeptide comprises the amino acid sequence of any one of SEQ ID NO: 179-186 fused to any one of SEQ ID NO: 79-86.

77. The modified BoNT polypeptide of any one of claims 64-76 further comprising a modified linker region.

78. The modified BoNT polypeptide of claim 77, wherein the modified linker region comprises a protease cleavage site.

79. The modified BoNT polypeptide of claim 78, wherein the protease cleavage site comprises the amino acid sequence of any one of SEQ ID NO: 206-212.

80. A nucleic acid molecule comprising a polynucleotide encoding a modified BoNT polypeptide of any one of claims 1-76.

81. A nucleic acid vector comprising the nucleic acid molecule of claim 80.

82. A cell comprising the nucleic acid molecule of claim 80 or the nucleic acid vector of claim 81.

83. A cell expressing the modified BoNT polypeptide of any one of claims 1-76.

84. A method of producing a modified BoNT polypeptide, the method comprising the steps of culturing the cell of claim 82 or claim 83 under conditions wherein the modified BoNT polypeptide is produced.

85. The method of claim 84, further comprising recovering the modified BoNT polypeptide from the culture.

86. A pharmaceutical composition comprising the modified BoNT polypeptide of any one of claims 1-76.

87. The pharmaceutical composition of claim 86, further comprising a pharmaceutically acceptable excipient.

88. A kit comprising a pharmaceutical composition of claim 86 or claim 87 and directions for therapeutic administration of the pharmaceutical composition.

89. A method of treating a condition, the method comprising administering a therapeutically effective amount of the modified BoNT polypeptide of any one of claims 1-76, or the pharmaceutical composition of or claim D1 or D2 to a subject to treat the condition.

90. The method of claim 89, wherein the condition is associated with overactive neurons or glands.

91. The method of claim 90, wherein the condition is selected from the group consisting of:
spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, dermatological or aesthetic/cosmetic conditions, obesity/reduced appetite, depression.

92. The method of claim 89 or 90, wherein the condition is not associated with unwanted neuronal activity.

93. The method of claim 92, wherein the condition is selected from the group consisting of: psoriasis, allergy, haemophagocytic lymphohistiocytosis, and alcoholic pancreatic disease.

94. The method of any one of any one of claims 89-93, wherein the administering is via injection to where unwanted neuronal activity is present.

95. The modified BoNT polypeptide of any one of claims 1-76 or the pharmaceutical composition of claim D1 or D2, for use in treating a condition associated with unwanted neuronal activity.

96. The modified BoNT polypeptide of any one of claims 1-76, or the pharmaceutical composition of claim 86 or 87, for use in medicine.

97. The modified BoNT polypeptide of any one of claims 1-76, or the pharmaceutical composition of claim 86 or 87, for cosmetic use.
